SEQUENCE LISTING (9 sequences)

SEQ ID NO: 1 - DNA, 40 bp, Artificial Sequence
  Sequence of artificial origin suitable for practice of the invention as described and specific to no particular biological species
ctggatttcctcagcgacgtgcgtgccattaaagtgcgac

SEQ ID NO: 2 - DNA, 15 bp, Artificial Sequence
  Sequence of artificial origin suitable for practice of the invention as described and specific to no particular biological species
gctgaggaaatccag

SEQ ID NO: 3 - DNA, 14 bp, Artificial Sequence
  Sequence of artificial origin suitable for practice of the invention as described and specific to no particular biological species
tggttgactgcgat

SEQ ID NO: 4 - DNA, 57 bp, Homo sapiens
  misc_feature (1)..(57): Polynucleotide template
gaacgttcactccatcggtcagtaccgcatcgacgctggtaaaaccgttccgatcgt

SEQ ID NO: 5 - RNA, 22 nt, Artificial Sequence
  Sequence of artificial origin suitable for practice of the invention as described and specific to no particular biological species
cuggauuuccucagcagcaccg

SEQ ID NO: 6 - RNA, 15 nt, Artificial Sequence
  Sequence of artificial origin suitable for practice of the invention as described and specific to no particular biological species
gcugaggaaauccag

SEQ ID NO: 7 - DNA, 60 bp, Artificial Sequence
  Sequence of artificial origin suitable for practice of the invention as described and specific to no particular biological species
cagcgtgaaattaggctgagaacagaatgattgatggtatcttttaggaacctttaggtc

SEQ ID NO: 8 - DNA, 16 bp, Artificial Sequence
  Sequence of artificial origin suitable for practice of the invention as described and specific to no particular biological species
taaaagataccatcaa

SEQ ID NO: 9 - DNA, 12 bp, Artificial Sequence
  Sequence of artificial origin suitable for practice of the invention as described and specific to no particular biological species
tcattctgttct

(19) United States
(12) Patent Application Publication
(10) Pub. No.: US 2010/0029008 A1
(43) Pub. Date: Feb. 4, 2010

(54) POLYMERASE-INDEPENDENT ANALYSIS OF THE SEQUENCE OF POLYNUCLEOTIDES

(75) Inventors: Jan André Rojas Stütz, Atizapan de Zaragoza (MX); Eric Kervio, Stutensee (DE); Clemens Richert, Waldbronn (DE); Patrizia Hagenbuch, Karlsruhe (DE); Annette Hochgesand, Linkenheim-Hochstetten (DE); Niels Griesang, Kelstesbach (DE); Stephanie Vogel, Philipsburg (DE); Ulrich Plutowski, Titisee-Neustadt (DE)

Correspondence Address: ROTHWELL, FIGG, ERNST & MANBECK, P.C., 1425 K STREET, N.W., SUITE 800, WASHINGTON, DC, 20005, US

(73) Assignee: FEBIT BIOTECH GMBH, Heidelberg (DE)

(21) Appl. No.: 11/721,914

(22) PCT Filed: Dec. 12, 2005
(86) PCT No.: PCT/EP2005/013062

§ 371 Date: Jun. 15, 2007

(30) Foreign Application Priority Data
Dec. 16, 2004 (EP) 04029858.0

(51) Int. Cl. G01N 33/00; C07H 1/00; C07H 19/10; C07H 19/20

(52) U.S. Cl. 436/94; 536/25.3; 536/25.32; 536/26.8; 536/26.7

(57) ABSTRACT

The present invention concerns methods of polymerase independent template directed elongation of polynucleotides, nucleotide building blocks used in these methods as well as the use of the methods and building blocks for the determination of nucleotide sequences, in particular for the determination of SNPs, base modifications, mutations, rearrangements and methylation patterns.

The present invention concerns methods of polymerase-independent template-directed elongation of polynucleotides, nucleotide building blocks used in these methods as well as the use of these methods and building blocks for the determination of nucleotide sequences, including the determination of SNPs, base modifications, mutations, rearrangements and methylation patterns.

### BACKGROUND OF THE INVENTION

The ability to determine the nucleotide sequence of naturally occurring nucleotide chains, i.e. DNA and RNA, has been one of the major breakthroughs in understanding the function of proteins and the processes of regulation within cells and organisms. The first of such methods was developed by Sanger F. and A. R. Culson (1975; J. Mol. Biol. 94: 444-448) and was based on the elongation of DNA chains with DNA polymerase. An equally powerful method based on the chemical degradation of DNA chains was developed by Maxam A. and Gilbert W. (1977; Proc. Natl. Acad. Sci. U.S.A. 74: 560-564). Sanger later devised a second method for sequencing DNA and again used an enzymatic rather than a chemical technique employing specific terminators of DNA chain elongation, i.e. 2′,3′-dideoxynucleoside triphosphates, which could be incorporated normally into a growing DNA chain through their 5′-triphosphate groups (Sanger F. S. et al. (1997) Proc. Natl. Acad. Sci. USA 74: 5463-5467). This method, which was also called dideoxy chain termination method, is the method which has been most widely used. Using this method it has been possible in recent years to determine the sequence of billions of nucleotides of genomic, plasmid, or viral DNA of a wide variety of organisms. A schematic representation of such a polymerase-based sequence analysis is depicted in FIG. 1. In an initial step of the dideoxy chain termination method an oligonucleotide primer is annealed to a single stranded nucleic acid template and in four separate reactions reaction mixes comprising all four deoxynucleotides, i.e. dA, dC, dG and dT, but only one type of dideoxynucleotide for each reaction, i.e. either ddA, ddC, ddG or ddT, and DNA-polymerase are added to the template and the annealed primer and the primer is extended along the template. The extension reaction is terminated once a dideoxynucleotide is added to the growing nucleic acid chain, thus generating in each reaction mix reaction products of various length, wherein the length of each product in, for example, the ddA reaction mix corresponds to the respective position of every dT nucleotide 3′ of the primer in the template.

Over the years several modifications and improvements of this general sequencing strategy have been developed. For example, the detection of the reaction products was carried out initially by radioactively labelling of the incorporated nucleotides or the primer and the extension products were separated by polyacrylamide gel electrophoresis, however, later methods have used fluorescently labelled dideoxynucleotides and mass spectrometry to determine the identity of the last incorporated dideoxynucleotide (see, for example, Lechner D. et al. (2001) Curr. Opin. Chem. Biol. 6: 31-38), Housby, J. N. (ed.) (2001) Mass Spectrometry and Genomic Analysis, Kluwer, Erdogan F. et al. (2001) Nucleic Acids. Res. 29: E36).

The determination of the sequence of long stretches of nucleotide sequences or even whole genomes is still one of the major foci of large sequencing projects like, for example, the human genome project. However, it has been realized that while organisms of the same species share a tremendous amount of sequence homology that there are significant differences between individuals, which account for the diversity seen within a species. These differences include the mutation or deletion of single base pairs, rearrangements and the determination of modifications of the core bases by, for example, methylation. In particular SNPs (single nucleotide polymorphisms) have gained attention since they have an abundance of one SNP per 500 to 1,000 base pairs and account for about 90% of all differences in genetic information between two individuals of the same species. The determination of SNP patterns should lead to a better understanding of phenotypes of individuals including the differences in susceptibility to various diseases and/or the side effects of various medications (Twyman R. M., Primrose S. B. (2003) Pharmaco Genomics, 4: 67-79). To reliable correlate phenotypes with genotypes it is necessary to determine a large number of SNPs. To that end a “single nucleotide polymorphism consortium” has been formed wherein a large number of companies and government institutions collaborate (Thorisson, G. A. and Stein L. D. (2003) Nucleic Acids Res. 31: 124-127). SNP genotyping holds the promise of answering both fundamental biological questions and of obtaining information which allows individualized medicine.

The basic enzymatic sequencing reaction developed by Sanger et al. has been further modified in recent years to facilitate the rapid determination of, for example, SNPs. The company Sequenom has developed a method wherein the primer extension is carried out on the surface of a chip and the sequence determination is carried out automatically using mass spectrometry. This method which has been called “MassARRAY” has found wide use and has replaced the “TaqMan allelic discrimination assay” developed by Applied Biosystems and the “HuSNP Human Genome Scans” developed by Affymetrix. Another method has been developed by Variagenics (NuCleave™) which is based on a mass spectrometric read out after a fragmenting process, which is induced by a chemically modified nucleotide. Other methods involve the so-called “Invader assays” (see, for example, Griffin T. et al. (1999) Proc. Natl. Acad. Sci. USA, 96: 6301-6306 and Oliver N. et al (2002) Nucleic Acids Res. 30: E53) and the PNA/cyanine technique of Norden and Zare (Wilhelmsson L. M. et al. (2002) Nucleic Acids Res. 30: E3).

It has also been discovered that 5-methylcytosine is the most frequent covalent base modification in the DNA of eukaryotic cells. This modification plays a role, for example, in the regulation of the transcription, in genetic imprinting, and in tumorigenesis. Therefore, the identification of 5-methylcytosine as a component of genetic information is of considerable interest. However, 5-methylcytosine positions cannot directly be identified by sequencing since 5-methylcytosine has the same base pairing behaviour as cytosine. Moreover, the epigenetic information carried by 5-methylcytosine is completely lost during PCR amplification. In view of this problem the most frequently used method for analysing DNA for 5-methylcytosine is based upon the specific reaction of bisulfite with cytosine which, upon subsequent alkaline hydrolysis, is converted to uracil which corresponds to thymidine in its base pairing behaviour. However, 5-methylcytosine remains unmodified under these conditions. Consequently, the original DNA is converted in such a manner that methylcytosine, which originally could not be distinguished from cytosine by its base pairing behaviour, can now be detected as the only remaining cytosine using “normal” molecular biological techniques, for example, by sequencing or by amplification and hybridisation (see, for example, Olek A et al. (1996) Nucleic Acids Res. 24:5064-6). Using this method it has been possible to analyse the methylation pattern of cytosines in individual cells. An overview of further known methods of detecting 5-methylcytosine may be gathered from the following review article: Rein, T. et al. (1998) Nucleic Acids Res. 26: 2255. There has been an effort to identify the methylation pattern, i.e. the epigenetic information, of the human genome similar to the human genome project. This effort is based on enzymatic sequencing of bisulfite modified genomic DNA.

All enzyme-based sequencing or extension assays described above share certain disadvantages. For example, the reactions can only be carried out in an environment which leaves the enzyme functional, e.g. it is not possible to add or leave out certain salts, detergents, solvents or other substances, which might be desirable to improve the stringency of base pairing or to achieve a more cost effective process. The respective polymerase used for extension puts further limitations on the reaction since it will only accept natural nucleotides or nucleotides, which are only modified to a small extent. Bulky side chains, which might be desirable for certain detection methods, can often not be attached to nucleotides without inhibiting the enzyme activity. For example, mass sensing which could be used to detect the mass of a reaction product requires that the mass difference between two species is larger than about 1000 g/mol, thus, the bulky nucleotide side chains, which would be required to employ mass sensing will often prevent their incorporation into the extension product. In addition enzyme based methods require the provision of expensive polymerases and of nucleotide triphosphates. Mass spectrometric analysis of elongation reactions requires a purification step prior to acquiring the spectra of the products.

In the prior art non-enzymatic primer extension reactions and attempts to replicate nucleic acids have been carried out in the context of research trying to elucidate dealing with the origin of life (“prebiotic chemistry”). Such non-enzymatic primer extension reactions have been described in particular by the groups of Orgel (see, for example Zilinsky, W. S, and Orgel L. E. (1987) Nucleic Acids Res. 15: 1699-1715) and Kiedrowski (see, for example Luther A. et al. (1998) Nature, 396: 245-248), Kanavarioti (see, for example Kanavarioti, A. et al. (1995) J. Org. Chem. 60: 632-637) and Goebel (Kurz, M. et al. (1998) Helv. Chim. Acta. 81: 1156-1180). None of these extension reactions has been used to analyse the sequence of genetic material. The primary reasons for this were that (i) the art known non-enzymatic primer extension reactions occurred so slowly, e.g. t_{1/2} in excess of 18 h, that it was untenable to use them even for single base pair non-enzymatic extension, (ii) successful reactions require concentrations of template strands that demand quantities of genetic material far beyond what can be obtained by routine medical procedures and (iii) the art known template-directed non-enzymatic primer extension reactions showed a low specificity, i.e. the percentage of incorporated nucleotides not according to the base pairing rules of Watson and Crick, (A should pair with U or T and G with C) was high such that the reaction product of the extension step only provided inaccurate information on the corresponding nucleotide on the template strand. Methods for detecting which nucleotide was appended to the primer in template-directed reactions employing a mixture of all four activated nucleotides (activated A, C, G and T or U) have not been demonstrated before.

### DESCRIPTION OF THE INVENTION

The present inventors have now identified novel activated nucleotides, which can be employed in a template directed extension of oligonucleotide with a free amino group at its 2′, 3′, or 5′ terminus without enzymatic catalysis. These nucleotides and extension processes using them avoid several of the limitations of enzymatic processes of the prior art. For example, they do not require nucleotide triphosphates as building blocks and it is possible to use nucleotide derivates which would not be accepted by the active site of a polymerase. Consequently, the novel nucleotides allow a much higher flexibility in the choice of the nucleotide or nucleotide derivative employed. A further advantage of the use of the nucleotides of the present invention is that polynucleotides resulting from enzyme-free extension reactions can be analyzed with less preparation of the extension product and are, thus, more amenable to rapid direct analysis by, for example, mass spectrometry without purification steps. The template-directed reactions occur with high fidelity.

Accordingly, a first aspect of the present invention is a nucleotide having a structure according to formula (I)

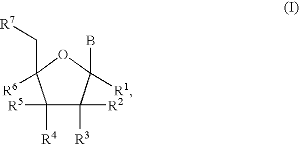

wherein
R¹ has the meaning H; saturated or unsaturated, linear or branched, C₁ to C₁₀ alkyl, e.g. C₁, C₂, C₃, C₄, CS, C₆, C₇, C₈, C₉ or C₁₀ alkenyl, in particular methyl, ethyl, n-propyl, iso-propyl, n-butyl, tert-butyl, or pentyl, C₁, C₂, C₃, C₄, C₈, C₆, C₇, C₈, C₉ or C₁₀ alkenyl, in particular ethenyl, 1-propenyl, 2-propenyl, butenyl, or pentenyl, C₁, C₂, C₃, C₄, C₈, C₆, C₇, C₈, C₉ or C₁₀ alkinyl, in particular ethinyl, 1-propinyl, 3-propinyl, butinyl, or pentinyl, and wherein the saturated or unsaturated C₁ to C₁₀ alkyl is preferentially substituted with one or more halogen, e.g. F, Cl, Br, or I, OH, SH, NR′R″; NR′R″; NH—CO—R; or is a direct or indirect link to a marker residue or a stacking residue; preferably R¹ has the meaning H, OH or is a direct or indirect link to a marker residue or a stacking residue;

R² has the meaning H; OH; SH; F; Cl; Br; I; saturated or unsaturated, linear or branched, C₁ to C₁₀ alkyl, e.g. C₁, C₂, C₃, C₄, C₅, C₆, C₇, C₈, C₉ or C₁₀ alkyl, in particular methyl, ethyl, n-propyl, iso-propyl, n-butyl, tert-butyl, or pentyl, C₁, C₂, C₃, C₄, C₅, C₆, C₇, C₈, C₉ or C₁₀ alkenyl, in particular ethenyl, 1-propenyl, 2-propenyl, butenyl, or pentenyl, C₁, C₂, C₃, C₄, C₅, C₆, C₇, C₈, C₉ or C₁₀ alkinyl, in particular ethinyl, 1-propinyl, 3-propinyl, butinyl, or pentinyl, and wherein the saturated or unsaturated C₁ to C₁₀ alkyl is preferentially substituted with one or more halogen, e.g. F, Cl, Br, I, OH, SH, NR′R″; NR′R″; NH—CO—R; or is a direct or indirect link to a marker residue or a stacking residue; preferably R² has the meaning H, OH or is a direct or indirect link to a marker residue or a stacking residue;

R³ has the meaning H; OH; SH; F; Cl, Br; I; saturated or unsaturated, linear or branched, unsubstituted or substituted C₁ to C₁₀ alkyl, e.g. C₁, C₂, C₃, C₄, C₅, C₆, C₇, C₈, C₉ or C₁₀ alkyl, in particular methyl, ethyl, n-propyl, iso-propyl, n-butyl, tert-butyl, or pentyl, C₁, C₂, C₃, C₄, C₅, C₆, C₇, C₈, C₉ or C₁₀ alkenyl, in particular ethenyl, 1-propenyl, 2-propenyl, butenyl, or pentenyl, C₁, C₂, C₃, C₄, C₅, C₆, C₇, C₈, C₉ or C₁₀ alkinyl, in particular ethinyl, 1-propinyl, 3-propinyl, butinyl, or pentinyl, and wherein the saturated or unsaturated C₁ to C₁₀ alkyl is preferentially substituted with one or more halogen, e.g. F, Cl, Br, or I, OH, SH, NR′R″; NR′R″; is a phosphate group; an activated phosphor ester; an activated carboxylic ester; CHO; COOH; a polynucleotide; a polynucleotide comprising a stacking residue; is a direct or indirect link to a marker residue or a stacking residue; or is connected to R⁶ via a C₁ to C₄ alkyl, e.g. methyl, ethyl, propyl, or butyl, or alkyl ether, e.g. methyl ether, ethyl ether, propyl ether, or butyl ether; preferably R³ has the meaning H; OH; NH₂; NHR′; NR′R″; an activated phosphor ester; activated carboxylic ester; or is a direct or indirect link to a marker residue or a stacking residue; more preferably R³ has the meaning H; OH; NH₂; or NHR′; most preferably R³ has the meaning H or OH;
R⁴ has the meaning H; OH; SH; F; Cl; Br; I; saturated or unsaturated, linear or branched, unsubstituted or substituted C₁ to C₁₀ alkyl, e.g. C₁, C₂, C₃, C₄, C₅, C₆, C₇, C₈, C₉ or C₁₀ alkyl, in particular methyl, ethyl, n-propyl, iso-propyl, n-butyl, tert-butyl, or pentyl, C₁, C₂, C₃, C₄, C₅, C₆, C₇, C₈, C₉ or C₁₀ alkenyl, in particular ethenyl, 1-propenyl, 2-propenyl, butenyl, or pentenyl, C₁, C₂, C₃, C₄, C₅, C₆, C₇, C₈, C₉ or C₁₀ alkinyl, in particular ethinyl, 1-propinyl, 3-propinyl, butinyl, or pentinyl, and wherein the saturated or unsaturated C₁ to C₁₀ alkyl is preferentially substituted with one or more halogen, e.g. F, Cl, Br, or I, OH, SH, NR′R″; NR′R″; is a phosphate group; an activated phosphor ester, an activated carboxylic ester; CHO; COOH; a polynucleotide; a polynucleotide comprising a stacking residue; or is a direct or indirect link to a marker residue or a stacking residue; preferably R⁴ has the meaning H′, OH′, NH_{2′}, NHR′, NR′R″; activated phosphor ester; activated carboxylic ester or is a direct or indirect link to a marker residue or a stacking residue; more preferably R⁴ has the meaning H; OH; NH₂; NHR′ activated phosphor ester; most preferably R⁴ has the meaning H; OH; or activated phosphor ester;
R⁵ has the meaning H; OH; SH; F; Cl; Br; I; saturated or unsaturated, linear or branched, C₁ to C₁₀ alkyl, e.g. C₁, C₂, C₃, C₄, C₅, C₆, C₇, C₈, C₉ or C₁₀ alkyl, in particular methyl, ethyl, n-propyl, iso-propyl, n-butyl, tert-butyl, or pentyl, C₁, C₂, C₃, C₄, C₅, C₆, C₇, C₈, C₉ or C₁₀ alkenyl, in particular ethenyl, 1-propenyl, 2-propenyl, butenyl, or pentenyl, C₁, C₂, C₃, C₄, C₅, C₆, C₇, C₈, C₉ or C₁₀ alkinyl, in particular ethinyl, 1-propinyl, 3-propinyl, butinyl, or pentinyl, and wherein the saturated or unsaturated C₁ to C₁₀ alkyl is preferentially substituted with one or more halogen, e.g. F, Cl, Br, or I, OH, SH, NR′R″; NR′R″; NH—CO—R; or is a direct or indirect link to a marker residue or a stacking residue; preferably R⁵ has the meaning H, OH or is a direct or indirect link to a marker residue or a stacking residue;
R⁶ has the meaning H; saturated or unsaturated, linear or branched, C₁ to C₁₀ alkyl, e.g. C₁, C₂, C₃, C₄, C₅, C₆, C₇, C₈, C₉ or C₁₀ alkyl, in particular methyl, ethyl, n-propyl, iso-propyl, n-butyl, tert-butyl, or pentyl, C₁, C₂, C₃, C₄, C₅, C₆, C₇, C₈, C₉ or C₁₀ alkenyl, in particular ethenyl, 1-propenyl, 2-propenyl, butenyl, or pentenyl, C₁, C₂, C₃, C₄, C₅, C₆, C₇, C₈, C₉ or C₁₀ alkinyl, in particular ethinyl, 1-propinyl, 3-propinyl, butinyl, or pentinyl, and wherein the saturated or unsaturated C₁ to C₁₀ alkyl is preferentially substituted with one or more halogen, e.g. F, Cl, Br, or I, OH, SH, NR′R″; NR′R″; NH—CO—R; is a direct or indirect link to a marker residue or a stacking residue or is connected to R⁴ via a C₁ to C₄ alkyl, e.g. methyl, ethyl, propyl or butyl, or alkyl ether, e.g. methyl ether, ethyl ether, propyl ether, or butyl ether; preferably R⁶ has the meaning H, OH or is a direct or indirect link to a marker residue or a stacking residue;
R⁷ has the meaning H; OH; SH; F; Cl; Br; I; saturated or unsaturated, linear or branched, unsubstituted or substituted C₁ to C₁₀ alkyl, e.g. C₁, C₂, C₃, C₄, C₅, C₆, C₇, C₈, C₉ or C₁₀ alkyl, in particular methyl, ethyl, n-propyl, iso-propyl, n-butyl, tert-butyl, or pentyl, C₁, C₂, C₃, C₄, C₅, C₆, C₇, C₈, C₉ or C₁₀ alkenyl, in particular ethenyl, 1-propenyl, 2-propenyl, butenyl, or pentenyl, C₁, C₂, C₃, C₄, C₅, C₆, C₇, C₈, C₉ or C₁₀ alkinyl, in particular ethinyl, 1-propinyl, 3-propinyl, butinyl, or pentinyl, and wherein the saturated or unsaturated C₁ to C₁₀ alkyl is preferentially substituted with one or more halogen, e.g. F, Cl, Br, I, OH, SH, NR′R″; NR′R″; is a phosphate group; an activated phosphor ester, an activated carboxylic ester; CHO; and COOH; polynucleotide; polynucleotide comprising a stacking residue; or is a direct or indirect link to a marker residue or a stacking residue; preferably R⁴ has the meaning H, OH, NR′R″, an activated phosphor ester, an activated carboxylic ester or is a direct or indirect link to a marker residue or a stacking residue; more preferably R⁴ has the meaning H, OH, NH₂ or NHR′; or activated phosphor ester most preferably R⁷ has the meaning activated phosphor ester;
wherein R has the meaning H; saturated or unsaturated, linear or branched, unsubstituted or substituted alkyl, in particular C₁ to C₁₀ alkyl e.g. C₁, C₂, C₃, C₄, CS, C₆, C₇, C₈, C₉ or C₁₀ alkyl, in particular methyl, ethyl, n-propyl, iso-propyl, n-butyl, tert-butyl, pentyl, C₁, C₂, C₃, C₄, C₅, C₆, C₇, C₈, C₉ or C₁₀ alkenyl, in particular ethenyl, 1-propenyl, 2-propenyl, butenyl, pentenyl, C₁, C₂, C₃, C₄, C₈, C₆, C₇, C₈, C₉ or C₁₀ alkinyl, in particular ethinyl, 1-propinyl, 3-propinyl, butinyl, pentinyl, and wherein the saturated or unsaturated C₁ to C₁₀ alkyl is preferentially substituted with one or more halogen, e.g. F, Cl, Br, or I, OH, SH, NR′R″; saturated or unsaturated, unsubstituted or substituted cycloalkyl, in particular C₃ to C₈ cycloalkyl e.g. cyclopropyl, cyclobutyl, cyclopentyl or cyclohexyl, or unsubstituted or substituted aryl or heteroaryl, and
R′ and R″ independent of each other have the meaning H; saturated or unsaturated, linear or branched, unsubstituted or substituted alkyl, in particular C₁ to C₁₀ alkyl, e.g. C₁, C₂, C₃, C₄, C₈, C₆, C₇, C₈, C₉ or C₁₀ alkyl, in particular methyl, ethyl, n-propyl, iso-propyl, n-butyl, tert-butyl, pentyl, C₁, C₂, C₃, C₄, C₅, C₆, C₇, C₈, C₉ or C₁₀ alkenyl, in particular ethenyl, 1-propenyl, 2-propenyl, butenyl, pentenyl, C₁, C₂, C₃, C₄, C₈, C₆, C₇, C₈, C₉ or C₁₀ alkinyl, in particular ethinyl, 1-propinyl, 3-propinyl, butinyl, pentenyl, and wherein the saturated or unsaturated C₁ to C₁₀ alkyl is preferentially substituted with one or more halogen, e.g. F, Cl, Br, or I, OH, SH, NH₂, NH-alkyl, or N-dialkyl; saturated or unsaturated, unsubstituted or substituted cycloalkyl; unsubstituted or substituted aryl or heteroaryl, or are taken together to form a saturated or unsaturated heterocycle; preferably R′ and R″ independent of each other mean H, methyl, ethyl, propyl, isopropyl or butyl; and
   B is a purine or pyrimidine base or base analog thereof or a purine, a pyrimidine or base analog thereof comprising a stacking residue and/or a marker residue; under the proviso that at least one of R³, R⁴, and R⁷ is an activated phosphor ester or an activated carboxylic ester and under the proviso that when R¹, R², R⁵, R⁶ is H, R³, R⁴ is OH and B is adenine, guanine, cylosine, thymine or cyacil than R⁷ is not phosphoro-2-methylimidazolid. In most embodiments the nucleotide of the present invention will comprise only one activated phosphor ester or one activated carboxylic ester.

In a preferred embodiment of the nucleotide of the present invention R¹, R², R⁵ and R⁶ have the meaning H. In a further preferred embodiment of the nucleotide of the present invention R³, R⁴ and R⁷ independent of each other have the meaning H, OH, NR′R″, activated phosphor ester, activated carboxylic ester or are a direct or indirect link to a marker residue under the proviso that one of R³, R⁴, and R⁷ is an activated phosphor ester or an activated carboxylic ester. More preferably R³, R⁴ and R⁷ have this meaning, if R¹, R², R⁵ and R⁶ have the meaning H.

The term “activated phosphor ester” or “activated carboxylic ester” is referring to a phosphate or carboxy group activated for coupling to an amino group by a leaving group. In one embodiment the phosphate group can be further substituted with substituents, e.g. alkyl chains. Phosphate groups can be activated in a way similar to the activation of carboxy groups for coupling to amino groups in peptide synthesis. For the purpose of the present invention it is preferred that activated phosphor esters or activated carboxylic esters are the result of the reaction of a pentafluorophenyl ester reagent, a phosphonium reagent, an aminium reagent, or an acid fluoride reagent and a nucleotide with a phosphate group or substituted phosphate group. Such activating reagents and reaction conditions to be used for activation are well known in the art of peptide synthesis (see for example, L. Carpino (1997) Methods in Enzymology 289: 104) and can all be employed to generate the nucleotide of the present invention comprising a phosphate linked coupling group.

Examples of particular preferred activating reagents are 2-chloro-1,1,3,3-tetramethyluronoium hexachloroantimonate (ACTU), O-(7-azabenzotriazol-1-yl)-1,1,3,3-tetramethyluronoium hexafluorophosphate (HATU), 2-(1H-benzotriazol-1-yl)-1,1,3,3-tetramethyluronoium hexafluorophosphate (HBTU), O-(1H-6-chlorobenzotriazole-1-yl)-1,1,3,3-tetramethyl-uronium hexafluorophosphate (HCTU), O-(7-azabenzotriazol-1-yl)-bis(pyrrolidin-1-yl)-methylium hexafluorophosphate (HAPyU), 2-(1H-benzotriazol-1-yl)-bis(pyrrolidin-1-yl)-methylium hexafluorophosphate (HBPyU), O-(1H-6-chlorobenzotriazole-1-yl)-bis(pyrrolidin-1-yl)-methylium hexafluorophosphate (HCPyU), 2-(1H-benzotriazole-1-yl)-1,1,3,3-tetramethyluronium tetrafluoroborate (TBTU), O-(1H-6-chlorobenzotriazole-1-yl)-1,1,3,3-tetramethyluronium tetrafluoroborate (TCTU), 2-(endo-5-norbornene-2,3-dicarboxymido)-1,1,3,3-tetramethyluronium tetrafluoroborate (TNTU), O-(1,2-dihydro-2-oxo-pyridyl]-N,N,N′,N′-tetramethyluronium tetrafluoroborate (TPTU), 2-succininido-1,1,3,3-tetramethyl-uronium hexafluorophosphate (HSTU), 2-succinimido-1,1,3,3-tetramethyluronium tetrafluoroborate (TSTU), pentafluorophenol-tetramethyluronium hexafluorophosphate (PFTU), N,N,N′,N′-tetramethyl-fluoroformamidinium hexafluorophosphate (TFFH), N,N,N′,N′-tetramethyl-chloroformamidinium hexafluorophosphate (TCFH), bis(tetramethylene) fluoroformamidinium hexafluorophosphate (BTFFH), O-(cyano-(ethoxycarbonyl)-methylenamino)-1,1,3,3-tetramethyluronium tetrafluoroborate (TOTU), N-hydroxy-5-norbornene-endo-2,3-dicarboxamide (HONB), pentafluorophenyl trifluoroacetat, pentafluorophenyl diphenylphosphinate (FDPP), (PfPyU), (PfTU), O-(7-azabenzotriazol-1-yl)-tris(dimethylamino)-phosphonium hexafluorophosphate (AOP), 2-(1H-benzotriazol-1-yl)-tris(dimethylamino)-phosphonium hexafluorophosphate (BOP), O-(1H-6-chlorobenzotriazole-1-yl)-tris-(dimethylamino)phosphonium hexafluorophosphate (COP), 7-azobenzotliazolyoxy-tris(pyrrolidino)-phosphonium hexafluorophosphate (PyAOP), 1-benzotriazolyoxy-tris(pyrrolidino)-phosphonium hexafluorophosphate (PyBOP), and tris(pyrrolidino)-phosphonium hexafluorophosphate (PyCOP). Particularly preferred activating agents are HATU, HBTU and HCTU. Similarly, if the nucleotides of the present invention comprise a carboxylic or aldehyde group, these groups can also be activated with the above indicated activating reagents, i.e. pentafluorophenyl ester reagent, a phosphonium reagent, an uronium reagent, or an acid fluoride reagent are the preferred activating reagents.

The term “polynucleotide” as used in the context of the nucleotide of the present invention refers to a nucleotide chain with two or more, preferably 2, 3, 4, 5, 6, 7, 8, 9 or 10 nucleosides linked by phosphate and/or amid links, i.e. are RNA, DNA or PNA chains or mixtures thereof. The inclusion of one or more additional nucleotides at positions R³, R⁴ and/or R⁷, in particular if they are capable of base specific pairing with bases in the template strand adjacent to the first base will improve the interaction of the nucleotide with the template strand. This can improve the speed of the reaction, however, the length of the additional nucleotides should generally not exceed 10 nucleotides or otherwise the specificity of the coupling step will depend less on the nucleotide at the terminus, i.e. the one that is coupled to the polynucleotide primer, but rather on the flanking nucleotides.

The rate of the reaction of a nucleotide of the present invention with the free amino terminus of the polynucleotide primer to be extended, will generally depend on the activated phosphor ester present in the nucleotide. It has been found by the present inventors, that certain activated phosphor esters are particularly suitable because they facilitate a rapid completion of the coupling reaction, and thus, in a preferred embodiment of the nucleotide of the present invention the activated phosphor ester is selected from the group consisting of structures according to formulas (II) to (XIX)

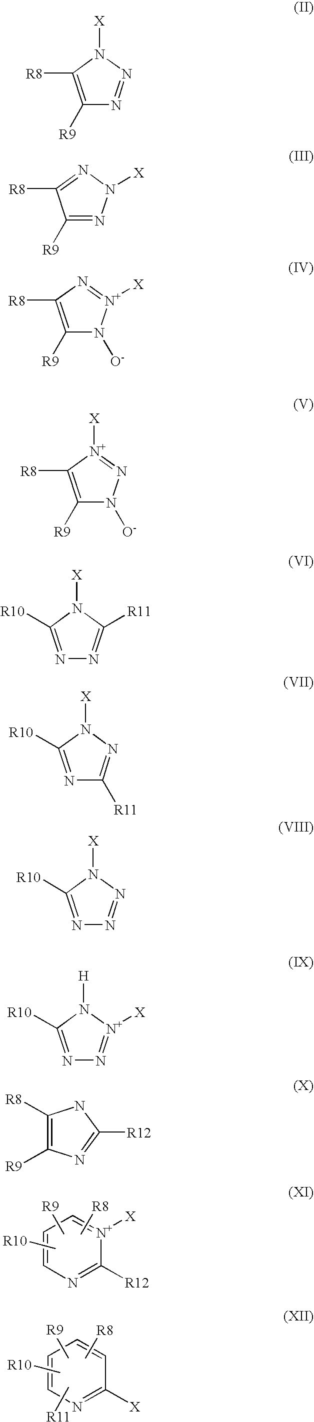

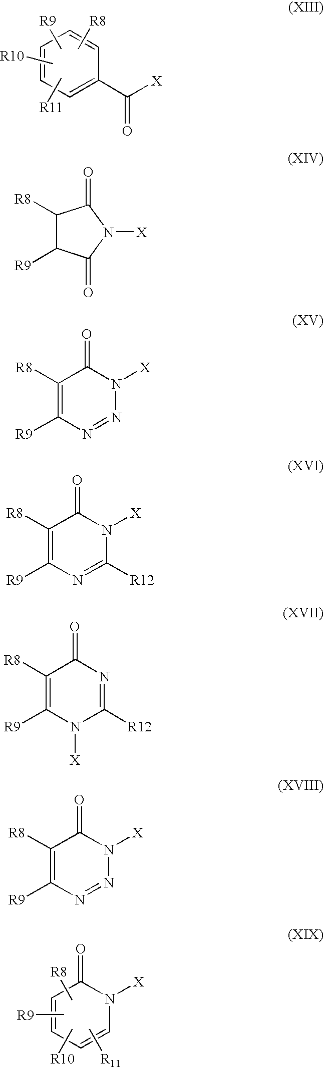

wherein R⁸ and R⁹ independent of each other have the meaning H; OH; SH; F; Cl; Br; I; CN; NO₂; saturated or unsaturated, linear or branched, unsubstituted or substituted C₁ to C₅ alkyl, e.g. C₁, C₂, C₃, C₄ or C₅ alkyl, in particular, methyl, ethyl, n-propyl, iso-propyl, n-butyl, tert-butyl, pentyl, C₁, C₂, C₃, C₄ or C₅ alkenyl, in particular ethenyl, 1-propenyl, 2-propenyl, butenyl, pentenyl, C₁, C₂, C₃; C₄ or C₅ alkinyl, in particular, ethinyl, 1-propinyl, 2-propinyl butinyl or pentinyl; or taken together form a saturated or unsaturated, unsubstituted or substituted mono, bi or polycyclic ring, in particular an aryl or heteroaryl substituted with one or more, preferably one, two three, or four substituents selected from the group consisting of OH; SH; F; Cl; Br; I; CN; NO₂.,
R¹⁰ and R¹¹ independent of each other have the meaning H; OH; SH; F; Cl; Br; I; CN; NO₂; saturated or unsaturated, linear or branched, unsubstituted or substituted C₁ to C₈ alkyl, e.g. C₁, C₂, C₃, C₄ or C₅ alkyl, in particular, methyl, ethyl, n-propyl, iso-propyl, n-butyl, tert-butyl, pentyl, C₁, C₂, C₃, C₄ or C₅ alkenyl, in particular ethenyl, 1-propenyl, 2-propenyl, butenyl, pentenyl, C₁, C₂, C₃; C₄ or C₅ alkinyl, in particular, ethinyl, 1-propinyl, 2-propinyl butinyl or pentinyl;
R¹² has the meaning H; OH; SH; F; Cl; Br; I; CN; NO₂; CH₃; substituted methyl; saturated or unsaturated, linear or branched, unsubstituted or substituted C₂ to C₈ alkyl, e.g. C₂, C₃, C₄ or C₅ alkyl, in particular, methyl, ethyl, n-propyl, iso-propyl, n-butyl, tert-butyl, pentyl, C₂, C₃, C₄ or C₅ alkenyl, in particular ethenyl, 1-propenyl, 2-propenyl, butenyl, pentenyl, C₂, C₃; C₄ or C₅ alkinyl, in particular, ethinyl, 1-propinyl, 2-propinyl butinyl or pentinyl,
and X is selected from the group consisting of the structures according to formulas (X) to (XXVII)

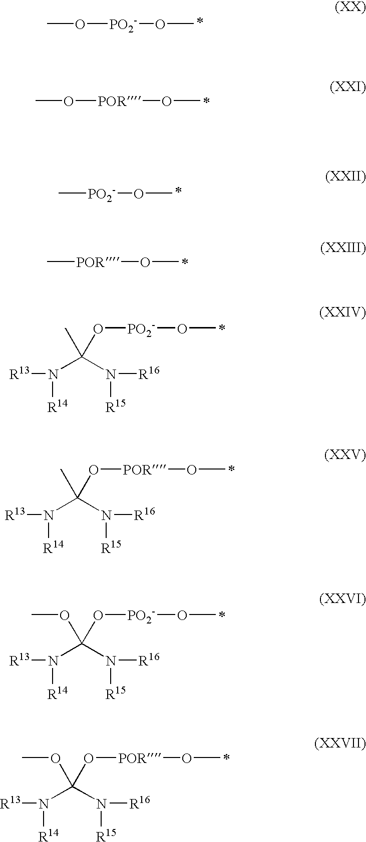

wherein * designates the bond of the phosphate group to the sugar moiety within the nucleotide,
R¹³ and R¹⁶ independent of each other have the meaning H; linear or branched, substituted or unsubstituted C₁ to C₁₀ alkyl; e.g. C₁, C₂, C₃, C₄, C₅, C₆, C₇, C₈, C₉ or C₁₀, in particular methyl, ethyl, n-propyl, iso-propyl, a-butyl, iso-butyl, tert-butyl, arpentyl, linear or branched C₁ to C₁₀ alkyl-NR¹⁷R¹⁸, wherein R¹⁷ and R¹⁸ independent of each other mean saturated or unsaturated, linear or branched substituted or unsubstituted C₁ to C₅ alkyl, e.g. C₁, C₂, C₃, C₄ or C₅ alkyl, in particular, methyl, ethyl, n-propyl, iso-propyl, n-butyl, tert-butyl, pentyl, C₁, C₂, C₃, C₄ or C₅ alkenyl, in particular ethenyl, 1-propenyl, 2-propenyl, butenyl, pentenyl, C₁, C₂, C₃; C₄ or C₅ alkinyl, in particular, ethinyl, 1-propinyl, 2-propinyl butinyl or pentinyl, C₃ to C₈ cycloalkyl, e.g. C₃, C₄, C₅, C₆, C₇, or C₅ cycloalkyl, aryl, or heteroaryl;
R¹⁴ and R¹⁵ either mean a free electron pair or R¹³ and R¹⁴ and/or R¹⁵ and R¹⁶ together form a heteroaryl, in particular pyridyl; and
R^{IIII} has the meaning saturated or unsaturated, linear or branched alkyl e.g. C₁, C₂, C₃, C₄, C₅, C₆, C₇, C₈, C₉ or C₁₀ alkyl, in particular methyl, ethyl, n-propyl, iso-propyl, n-butyl, tert-butyl, or pentyl, C₁, C₂, C₃, C₄, C₅, C₆, C₇, C₈, C₉ or C₁₀ alkenyl, in particular ethenyl, 1-propenyl, 2-propenyl, butenyl, or pentenyl, C₁, C₂, C₃, C₄, C₅, C₆, C₇, C₈, C₉ or C₁₀ alkinyl, in particular ethinyl, 1-propinyl, 3-propinyl, butinyl, or pentinyl, and wherein the saturated or unsaturated C₁ to C₁₀ alkyl is preferentially substituted with one or more halogen, e.g. F, Cl, Br, or I, OH, SH, NR′R″; NR′R″; aryl or heteroaryl, which can be substituted one or more times with OH, SH, NH₂, F, Cl, Br or I; and wherein R′ and R″ have the meaning and preferred meanings as outlined above.

The term “aryl” as used above preferably refers to an aromatic monocyclic ring containing 6 carbon atoms, an aromatic bicyclic ring system containing 10 carbon atoms or an aromatic tricyclic ring system containing 14 carbon atoms. Examples are phenyl, naphtalenyl or anthracenyl. The aryl group is optionally substituted. The term “heteroaryl” preferably refers to a five or six-membered aromatic monocyclic ring wherein at least one of the carbon atoms are replaced by 1, 2, 3, or 4 (for the five membered ring) or 1, 2, 3, 4, or 5 (for the six membered ring) of the same or different heteroatoms, preferably selected from O, N and S; an aromatic bicyclic ring system wherein 1, 2, 3, 4, 5, or 6 carbon atoms of the 8, 9, 10, 11 or 12 carbon atoms have been replaced with the same or different heteroatoms, preferably selected from O, N and S; or an aromatic tricyclic ring system wherein 1, 2, 3, 4, 5, or 6 carbon atoms of the 13, 14, 15, or 16 carbon atoms have been replaced with the same or different heteroatoms, preferably selected from O, N and S. Examples are oxazolyl, isoxazolyl, 1,2,5-oxadiazolyl, 1,2,3-oxadiazolyl, pyrrolyl, imidazolyl, pyrazolyl, 1,2,3-triazolyl, thiazolyl, isothiazolyl, 1,2,3,-thiadiazolyl, 1,2,5-thiadiazolyl, pyridinyl, pyrimidinyl, pyrazinyl, 1,2,3-triazinyl, 1,2,4-triazinyl, 1,3,5-triazinyl, 1-benzofuranyl, 2-benzofuranyl, indoyl, isoindoyl, benzothiophenyl, 2-benzothiophenyl, 1H-indazolyl, benzimidazolyl, benzoxazolyl, indoxazinyl, 2,1-benzosoxazoyl, benzothiazolyl, 1,2-benzisothiazolyl, 2,1-benzisothiazolyl, benzotriazolyl, quinolinyl, isoquinolinyl, quinoxalinyl, quinazolinyl, quinolinyl, 1,2,3-benzotriazinyl, or 1,2,4-benzotriazinyl.

If R⁸ and R⁹ are taken together to form a saturated or unsatured mono, bi or polyclyclic ring system in the context of the five-membered heteroaryls according to (II) to (V), (X) and (XIV) they preferably form a cyclopentadienyl, benzyl, napthyl, pyridinyl, pyrimidinyl, pyrazinyl, 1,2,3-triazinyl, 1,2,4-triazinyl and bicyclo[2.2.1]hepta-3-en.

If R⁸ and R⁹ are taken together to form a saturated or unsatured mono, bi or polyclyclic ring system in the context of the six-membered aryls or heteroaryls according to (X) to (XIII), (XV) to (XIX) furanyl, oxazolyl, isoxazolyl, 1,2,5-oxadiazolyl, 1,2,3-oxadiazolyl; pyrrolyl, imidazolyl, pyrazolyl, 1,2,3-triazolyl, thiazolyl, isothiazolyl, 1,2,3,-thiadiazolyl, 1,2,5-thiadiazolyl, or thiophenyl, pyridinyl, pyrimidinyl, pyrazinyl, 1,2,3-triazinyl; 1,2,4-triazinyl.

In a particularly preferred embodiment of the activated phosphor ester the phosphor ester is selected from the group consisting of compounds according to formulas (II) to (XIX). These leaving groups have a particularly short coupling time with terminal amino residues.

In a further preferred embodiment of the nucleotide of the present invention the activated phosphor ester is selected from a group consisting of structures according to formulas (XXVIII) to (XXXIX)

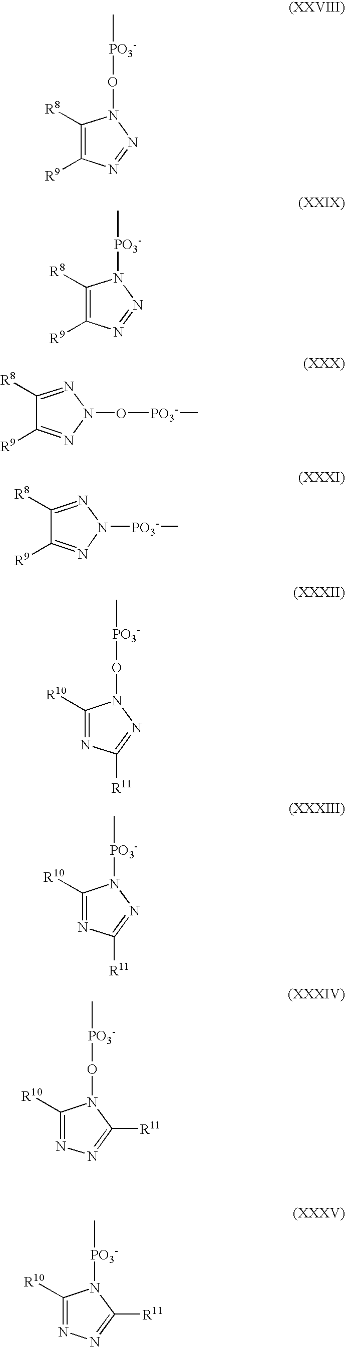

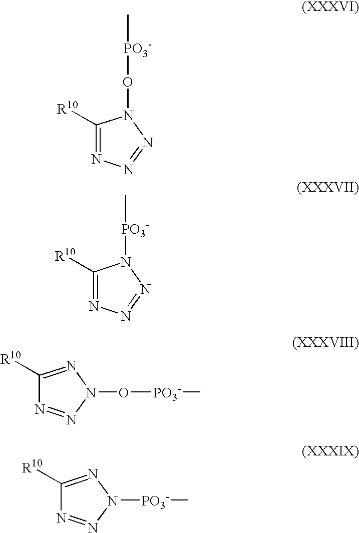

wherein R⁸ and R⁹ independent of each other have the meaning H; OH; SH; NH₂; F; Cl; Br; I; CN; NO₂; saturated or unsaturated, linear or branched, unsubstituted or substituted C₁ to C₅ alkyl, e.g. C₁, C₂, C₃, C₄, or C₅ alkyl, in particular methyl, ethyl, n-propyl, iso-propyl, n-butyl, tert-butyl, or pentyl, C₁, C₂, C₃, C₄, or C₅ alkenyl, in particular ethenyl, 1-propenyl, 2-propenyl, butenyl, or pentenyl, C₁, C₂, C₃, C₄, or C₅ alkinyl, in particular ethinyl, 1-propinyl, 3-propinyl, butinyl, or pentinyl, and wherein the saturated or unsaturated C₁ to C₅ alkyl is preferentially substituted with one or more halogen, e.g. F, Cl, Br, or I, OH, SH, NR′R″; NR′R″, wherein R′ and R″ have the meaning and preferred meaning as outlined above; or taken together form a saturated or unsaturated, unsubstituted or substituted mono, bi or polycyclic ring, in particular an aryl or heteroaryl substituted with one, two three, or four substituents selected from the group consisting of Cl and F;
and R¹⁰ and R¹¹ independent of each other have the meaning H; OH; SH; NH₂; F; Cl; Br; I; CN; NO₂; saturated or unsaturated, linear or branched, unsubstituted or substituted C₁ to C₅ alkyl e.g. C₁, C₂, C₃, C₄, or C₅ alkyl, in particular methyl, ethyl, n-propyl, iso-propyl, n-butyl, tert-butyl, or pentyl, C₁, C₂, C₃, C₄, or C₅ alkenyl, in particular ethenyl, 1-propenyl, 2-propenyl, butenyl, or pentenyl, C₁, C₂, C₃, C₄, or C₅ alkinyl, in particular ethinyl, 1-propinyl, 3-propinyl, butinyl, or pentinyl, and wherein the saturated or unsaturated C₁ to C₅ alkyl is preferentially substituted with one or more halogen, e.g. F, Cl, Br, or I, OH, SH, NR′R″; NR′R″, wherein R′ and R″ have the meaning and preferred meaning as outlined above.

If R⁸ and R⁹ are taken together to form a saturated or unsatured mono, bi or polyclyclic ring system in the context of the five-membered heteroaryls according to (XXVIII) to (XXXI) they preferably form a cyclopentadienyl, benzyl, napthyl, pyridinyl, pyrimidinyl, pyrazinyl, 1,2,3-triazinyl, 1,2,4-triazinyl and bicyclo[2.2.1]hepta-3-en.

In a preferred embodiment of the nucleotide of the present invention R⁸ and R⁹ together form an unsubstituted or substituted aromatic or heteroaromatic ring system, preferably a mono or bicyclic homo or heteroaromatic ring. Preferred ring structures are a benzole ring or an azabenzole ring. This ring can be substituted with one, two, three or four substituents, which are preferably selected from the group consisting of I, Cl, Br, I, NO₂ or CN.

Thus, in a preferred embodiment of the nucleotide of the present invention the activated phosphor ester with a structure according to:
a) formula (XVIII) is selected from the group consisting of 6-chloro-1-hydroxybenzotriazole phosphate, 1-hydroxybenzotriazole phosphate, 1-hydroxyazabenzotriazole phosphate and 1-hydroxytriazol phosphate;
b) formula (XXIX) is selected from the group consisting of benzotriazole phosphate, 6-chlorobenzotriazole phosphate, azabenzotriazole phosphate, and triazole phosphate;
c) formula (XXX) is selected from the group consisting of 6-chloro-2-hydroxybenzotriazole phosphate, 2-hydroxybenzotriazole phosphate, 2-hydroxyazabenzotriazole phosphate and 2-hydroxytriazol phosphate;
d) formula (XXXI) is selected from the group consisting of benzotriazole phosphate, 6-chlorobenzotriazole phosphate, azabenzotriazole phosphate, and triazole phosphate;
e) formula (XII) is selected from the group consisting of 1-hydroxytriazole phosphate, and 5-chloro-1-hydroxytriazole phosphate;
f) formula (XXXIII) is selected from the group consisting of triazole phosphate, 5-chloro-triazole phosphate;
g) formula (XIV) is selected from the group consisting of 1-hydroxytriazole phosphate, and 2-chloro-1-hydroxytriazole phosphate;
h) formula (XXXV) is selected from the group consisting of triazole phosphate, and 2-chloro-triazole phosphate;
i) formula (XXXVI) is selected from the group consisting of 1-hydroxytetrazole phosphate and 5-chloro-1-hydroxytetrazole phosphate;
j formula (XXXVII) is selected from the group consisting of tetrazole phosphate and 5-chloro-tetrazole phosphate;
k) formula (XXXVIII) is selected from the group consisting of 2-hydroxytetrazole phosphate and 5-chloro-2-hydroxytetrazole phosphate; and
l) formula (XXXIX) is selected from the group consisting of tetrazole phosphate and 5-chloro-tetrazole phosphate.

In another preferred embodiment of the nucleotide of the present invention the activating reagent is pentafluorophenole and, thus, the activated phosphate ester is pentafluorophenole phosphate.

The base of the nucleotide of the present invention can be any base, which is capable of base specific interaction with another base. Particular preferred bases or base analogs are bases or base analogs capable of specific interaction with naturally occurring bases, in particular with adenine (A), guanine (G), cytosine (C), thymine (T) and uracil (U). Theses bases are known to specifically form the following Watson-Crick base pairs: A-T, A-U and C-G. However, bases capable of stable interaction due to other types of specific base pairing comprising Hoogsteen base pairing, reverse Hoogsteen base pairing, reverse Watson-Crick base pairing, Wobble base pairing, reverse Wobble base pairing, homo purine base pairing, hetero purine base pairing or pyrimidine-pyrimidine base pairing can all equally be employed in a nucleotide of the present invention. A wide variety of bases and base pairs are known in the art, which are capable of base specific pairing. For a review of various bases capable of base specific pairing see Tinoco Jr. L. In Appendix 1 of: The RNA World (Gestland R. F. and Atlcins J. F., eds.); Cold Spring Harbor Laboratory Press, 1993, pp 603-607; Dirheimer G. et al. in: RNAs Structure, Biosynthesis and Function (D. Söll and U. RajBhandary, eds.); American Society for Microbiology, Washington, 1995, pp. 93-126 and G. A. Jeffrey and W. Sanger, Hydrogen Bonding in Biological Structures, Springer-Verlag, Berlin 1991.

As outlined above the type of base, which can be employed in the nucleotide of the present invention, is not limited by the fact that it has to fit into the reactive pocket of a polymerase, since the coupling reaction is carried out non-enzymatically. Thus, the skilled person is aware of various purine or pyrimidine bases or analogues thereof, which can all equally be employed in this invention. However, in a preferred embodiment of the nucleotide of the present invention the purine base is selected from the group consisting of adenine, deazaadenine, guanine, deazaguanosine, and inosine or from the respective purine base comprising a marker residue or stacking residue. Furthermore the preferred pyrimidine base is selected from the group consisting of cytosine, thymine, uracil, isocytosine, dihydrouracil, thiouracil, pseudouracil and 5-methylcytosine or from the respective pyrimidine base comprising a marker residue or stacking residue.

The purine or pyrimidine base does not only serve the purpose of allowing base specific interaction it can also introduce further functionalities into the nucleotide of the present invention. These functionalities, e.g. stacking residues or marker residues, can be attached to any residue within the purine or pyrimidine ring(s) in as long as it does not interfere with either the base specific interaction with another base and/or the coupling of the activated phosphor ester or carboxylic ester to the free amino primers of the polynucleotide primer with a free amino terminus, i.e. a 5′, 3′ or 2′ linked amino group. It is, however, preferred that the stacking residue or marker residue is attached to the 5-position of the pyrimidine or to the 7 or 8 position of the purine.

As pointed out above a wide variety of purine or pyrimidine bases can be used in the nucleotide of the present invention. In addition molecules are known, which are neither purines nor pyrimidines and which can still specifically interact with naturally occurring bases, in particular with A, G, C, T and U. These molecules are referred to as base analogues and can also be used in the nucleotide of the present invention. Preferred base analogs or analogs comprising a stacking residue or marker residue are selected from the group consisting of difluorotoluene and imidazole-4-carboxamide.

To increase the interaction of the nucleotide of the present invention with the template polynucleotide strand in order to facilitate and/or increase the speed of the coupling reaction it is preferred that a nucleotide of the present invention comprises a stacking residue. As used herein the term “stacking residue” refers to aromatic or hetero aromatic, mono, bi, tri, tetra or polycyclic ring systems capable of interacting with nucleobases. Preferentially the stacking residue is capable of sliding in between a bases on the polynucleotide template strand to which the polynucleotide primer has been annealed and, thus, facilitates or enhances base specific pairing between the nucleotide on the template strand and the nucleotide of the present invention, which is to be coupled to the polynucleotide primer. A wide variety of such stacking or nucleotide intercalating structures are known in the prior art and comprise in a preferred embodiment indole, napthol, anthraquinone, bile acid, quinoline, quinolone, stilbene, pyrene, a steroid ring system, an ethidium residue, an anthracene residue, and tetracene. These molecules can be substituted with one or more residues selected from the group consisting of OH, SH, NH₂, F, Cl, Br and I.

Once the nucleotide of the present invention has been coupled to a free amino group of a polynucleotide primer it is often desired to analyze the identity of the (last) nucleotide coupled to the polynucleotide primer. If this coupling was affected in a base specific manner it will be possible to derive the sequence of the template strand from the identity of the last coupled nucleotide. There is a wide variety of methods available to analyze primer extension products. One method is the analysis of the extension product by, for example, mass spectroscopy. This type of analysis is based on the fact, that all four nucleotides have a different molecular weight and, thus, depending on the respectively incorporated nucleotide the extension reaction leads to extension products with different masses. For this type of analysis it is usually not necessary to attach a marker to the nucleotide of the present invention, however, for various detection methods it is required that a marker residue are/(is) attached to the nucleotide of the present invention to allow specific detection of the nucleotide coupled to the polynucleotide primer. For example, it is possible to use four different fluorophores as marker residues and couple each to a different nucleotide, e.g. a nucleotide comprising a thymine, adenine, cytosine or guanine base. Consequently, the respective fluorescence of the resulting extension product will allow the determination of the type of nucleotide attached. In general any marker, which allows detection of the extension product by physical or chemical means, can be used in the context of the present invention, however, in a preferred embodiment the marker is selected from the group consisting of a fluorescent residue, a radioactive residue, a phosphorescent residue, a chelating residue comprising a metal ion and a quenching residue. A wide variety of such markers are known in the prior art.

In a preferred embodiment of the present invention the marker is a fluorescent dye. A large number of dyes are known, which can be used including alexa and cyanine dyes. Particularly preferred cyanine dyes are selected from the group consisting of carbocyanine, dicarbocyanine, and tricarbocyanine, e.g. Cy3, Cy5. Further dyes are discussed, which can be used in the context of the molecules of the present invention are described in Rosenblum et al. (1997) Nuc. Acids Res. 25:4500-4504. The synthesis of cyanine dyes can be carried out using the methods known in the state of the art and which are exemplified in, e.g. Hamer F. M. The Cyanine Dyes and Related Compounds, John Wiley and Sons, New York 1964; Ernst L A, et al. (1989) Cytometry 10:3-10; Southwick P L, et al., (1990) Cytometry 11:418-430; Lansdorp P M et al., (1991) Cytometry 12:723-730; Mujuumdor R B et al., (1993) Bioconjugate Chem. 4:105-11; Mujumdor S R et al., (1996) Bioconjugate Chem. 7:356-62; Flanagan J H et al., (1997) Bioconjugate Chem. 8:751-56; Keil D et al., (1991) Dyes and Pigments 17:19-27; Terpetschnig E and Lakowicz J R (1993) Dyes and Pigments 21:227-34; Terpetschnig E et al., (1994) Anal. Biochem. 217: 197-204; Lindsey J S et al., (1989) Tetrahedron 45:4845-66; Górecki T et al., (1996) J. Heterocycl. Chem. 33, 1871-6; Narayanan N and Patonay G (1995) J. Org. Chem. 60:2391-5, 1995; and Terpetschnig E et al., (1993) J. Fluoresc. 3:153-155. Additional processes are described in patent publications U.S. Pat. No. 4,981,977; U.S. Pat. No. 5,688,966; U.S. Pat. No. 5,808,044; EP 0 591 820 A1; WO 97/42976; WO 97/42978; WO 98/22146; WO 98/26077; and EP 0 800 831. Further suitable fluorophores, which can be detected simultaneously are depicted in Table 1.

TABLE 1

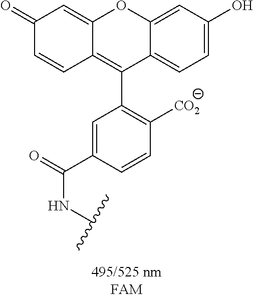

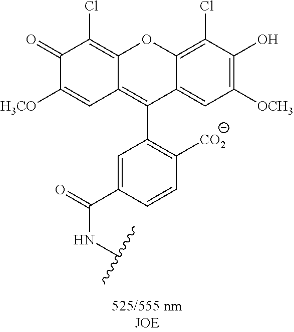

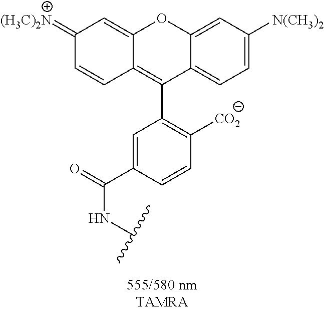

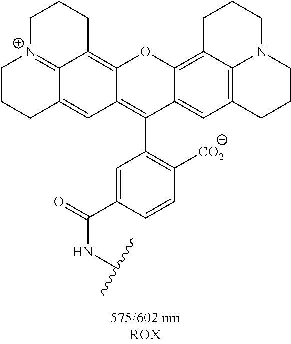

The marker residue can be attached to any part of the nucleotide of the present invention as long as it does not interfere with the coupling to the polynucleotide primer and/or the base specific interaction with the polynucleotide template. Preferably, as outlined above it is attached to the base but it can also be attached to the sugar backbone or to a further nucleotide or polynucleotide attached to the nucleotide of the present invention. When a quenching residue is used as a marker it is preferred that the corresponding polynucleotide primer comprises a fluorescent residue. Upon coupling of the nucleotide comprising a quenching residue the fluorescence of the fluorescent residue will be quenched and, thus, the read out will be the decrease in fluorescence. In the context of SNP or methylation analysis it is imaginable that two nucleotides are provided in a coupling reaction one of which carries a quenching residue and than the loss of fluorescence of the polynucleotide primer after the coupling reaction has been carried out is indicative of the presence of, e.g. a cytosine, which would be indicatives of the methylation of a cytosine in the underlying genomic sequence, which has been treated with bisulfite.

The term “direct link” to a stacking or marker residue as used throughout the specification means a covalent bond to a residue of the nucleotide, while the term “indirect link” as used herein means that one or more additional chemical residues which are connected by covalent or non-covalent bonds, preferentially by covalent bonds, are located between the nucleotide and the marker or stacking residue. These one or more additional chemical residues can also be termed “spacer” and can decrease the interference of the marker or stacking residue with the coupling reaction and/or the base specific pairing. In addition or alternatively the spacer can be photolabile. This allows the removal of the stacking or marker residue at any stage of the coupling reaction, if desired. Photolabile protection groups have been described in various publications including NVOC (Fodor et al. (191) Science 251: 767-773) MeNPOC, Pease et al. (1994) PNAS 91: 5022), Bochet (2002) J. Chem. Soc. Perkin Trans. I. 125-142 and Holmes (1997) J. Org. Chem. 62:2370-2380. Examples of photolabile protection groups, which can be used as photolabile spacers in the context of the present invention are disclosed in above publications and comprise in particular benzyl, o-nitrobenzyl, o-nitrophenylethyl, dieo-(nitrophenyl) and ethyloxy protection groups and derivatives thereof, e.g. NVOC, MeNPOC and NPPOC. If the methyl chain of the benzyl group is extended by one CH₂ radical the resulting protection groups are 2-(nitrophenyl)ethyl protection groups as disclosed, for example, in DE 44 44 996, DE 196 20 170, DE 198 58 440, U.S. Pat. No. 5,763,599, WO 00/35931, DE 199 52 113, WO 00/61594 und WO 02/20150. It is well known in the art how to synthesize nucleotides comprising photolabile protection groups without limitation all these methods can be employed. The marker and/or the stacking residue are then attached to the photolabile protection group in such that photo cleavage can occur and that the marker and/or stacking residue is concomitantly removed from the nucleotide.

If a marker or a stacking residue is attached to such a protection group it serves as a spacer between the nucleotide and the marker or stacking residue. The exposure to the relevant radiation spectrum will remove the marker. In a particularly preferred embodiment the photo induced removal of the spacer will reveal a free amino group or carboxy group for further coupling reactions. Preferably, the nucleotides of the present invention are provided with a 5′ or 3′ photolabile spacer in between the ribose and the marker. It is possible to subsequently add one nucleotide by one and determine in each case the respectively added nucleotide before the marker residue is cleaved of to expose a free amino or carboxy group, which can the be used to sequence specifically coupled one or more further nucleotides, which depending on the respective nucleotide coupled may have a different fluorescence. Thus, a series of steps comprising e.g. sequence specific coupling of one nucleotide from a group of differentially labelled nucleotides, measuring of fluorescence, photo deprotecting of the coupled nucleotide to expose a free amino or carboxy group, coupling of a further nucleotide, and measuring of fluorescence. This series will yield sequence information on two nucleotides 3′ or 5′ to the primer annealed to the template to be sequenced. The preferred nucleotides of the invention comprise a 2′, 3′ and/or 5′ prime photolabile protection group, which functions as a photocleavable spacer and which preferably protects either a carboxy or an amino functionality in the nucleotide. An example of a preferred sequencing method is depicted in FIG. 11 below.

In a further aspect the present invention relates to a method of polymerase independent elongation of a polynucleotide primer comprising the steps of:
a) providing a polynucleotide primer, with at least one 2′, 3′ or 5′ terminal amino group and
b) reacting the polynucleotide primer with a nucleotide of the present invention described above.

The term “polynucleotide primer” as used in the context of the methods of the present invention refers to a nucleotide chain with two or more, preferably 3, 4, 5, 6, 7, 8, 9, 10, 11, 12, 13, 14, 15, 16, 17, 18, 19, 20, 21, 22 23, 24, 25 nucleosides linked by phosphate and/or amid links, i.e. are RNA, DNA or PNA chains or mixtures thereof. Although the nucleotide primer can be of any length it is preferred that the length is between 5 and 50 bp, more preferably between 10 and 20 bp. If the primer is too long it is less likely to hybridize to a target sequence in a double stranded polynucleotide template in a hybridization reaction since its kinetic advantage over the reannealing reaction of the two strands of the double stranded polynucleotide template decreases. The polynucleotide primer can comprise additional chemical moieties like marker residues, e.g. a fluorescent moiety in cases where the nucleotide coupled to the nucleotide primer comprises a quenching residue.

The polynucleotide primer employed in the reaction can be in solution or can be linked directly or indirectly to a surface. If the polynucleotide primer is linked to a surface than it is preferred that the polynucleotide template and optionally a polynucleotide helper are provided in solution and are “captured” on the surface by the polynucleotide primer. Suitable surfaces are without limitation glass, metal, e.g. gold, plastic, e.g. Teflon®, polystyrol, polypropylene, polyethylene, polycarbonate, silicium oxide, and the like. The surface can have any three-dimensional shape. It can be flat or can be on a bead, e.g. SiO₂ or rubber coated magnetic bead, and can take on any shape suitable to allow the extension reaction to take place. If the surface is part of a chip it can additionally have inlet and outlet ports, flow lines, waste and buffer compartments, reaction chambers, e.g. DNA purification or PCR amplification chambers, as required and known in the art. Accordingly, the method of the present invention can also be carried out on a chip coated with one or more polynucleotide primers with a 2′, 3′, or 5′ terminal amino group. This chip can be packaged in a kit, which can optionally include one or more nucleotides of the present invention. The indirect link can be affected by another polynucleotide called polynucleotide capture probe, which is capable of non-covalent binding to the polynucleotide primer. Accordingly, the polynucleotide capture probe can be a nucleotide chain with two or more, preferably 3, 4, 5, 6, 7, 8, 9, 10, 11, 12, 13, 14, 15, 16, 17, 18, 19, 20, 21, 22 23, 24, 25 nucleosides linked by phosphate and/or amid links, i.e. are RNA, DNA or PNA chains or mixtures thereof. Although the capture probe can be of any length it is preferred that the length is between 5 and 50 bp, more preferably between 10 and 20 bp.

On one hand the method of the present invention can be carried out with the nucleotides of the present invention. However, due to the short shelf life of some of the nucleotides comprising an activated phosphor ester or activated carboxylic ester a further aspect of the present invention relates to a method of polymerase independent elongation of a polynucleotide primer comprising the steps of:
a) providing an polynucleotide primer, with at least one 2′, 3′ or 5′ terminal amino group and
b) reacting the polynucleotide primer with a nucleotide or a polynucleotide with at least one 2′, 3′ or 5′ terminal phosphate or carboxylic residue, preferentially phosphate residue, which has been activated with an activating reagent. In most instances the nucleotide or polynucleotide will only comprise one activatable phosphate or carboxy residue to assure that the coupling reaction with the polynucleotide primer occurs only at one position.

This method has the advantage that the reactive species can be generated immediately prior to the coupling reaction and, thus, suffers less from reduced shelf life. The term “activating reagent” refers to a reactive species that is capable of transferring a leaving group onto a 2′, 3′ or 5′ terminal phosphate or carboxy residue of a nucleotide or a polynucleotide. The activating reagents outlined above for activation of the nucleotides of the present invention can be employed in the method of the present invention. In a preferred embodiment of the method of the present invention the activating reagent is selected from a pentafluorophenyl ester reagent, a phosphonium reagent, an uronium reagent, or an acid fluoride reagent. Particular suitable activating reagents are selected from the group comprising ACTU, HATU, HBTU, HCTU, HAPyU, HBPyU, HCPyU, TBTU, TCTU, TNTU, TPTU, HSTU, TSTU, PFTU, TFFH, TCFH, BTFFH, TOTU, FDPP, PfPyU, PffU, AOP, BOP, COP, PyAOP, PyBOP, and PyCOP. Particular preferred activating reagents are HATU, HBTU and HCTU. The reaction conditions for carrying out activation reactions with the activating reagents outlined above are well established in the art of peptide synthesis and can be equally employed for the activation of the nucleotides or polynucleotides.

It is preferred that the nucleotide or polynucleotide having at least one 2′, 3′ or 5′ terminal phosphate or carboxy residue, preferentially phosphate residue, is activated prior to being contacted with the polynucleotide primer. The nucleotides or polynucleotides which are employed in this activating step have a 2′, 3′ or 5′ preferentially 3′ or 5′ terminal phosphate or carboxy residue. It is particularly preferred that the nucleotides are mono phosphates and are, thus, much cheaper than the nucleotide triphosphates commonly employed in enzyme based primer elongation reactions.

The type of nucleotide or polynucleotide that is employed in this activation reaction is not particular limited and, therefore, the nucleotide or polynucleotide can comprise any base capable of base specific interaction as set out above and in addition the nucleotide or polynucleotide can incorporate any additional residue(s) as set out above with respect to the nucleotides of the present invention, e.g. it can comprise one or more stacking residues and one or more marker residues. In a preferred embodiment of the method of the present invention a nucleotide or a dinucleotide is coupled to a terminal amino group of the polynucleotide primer. The term “polynucleotide” as used in this context refers to a nucleotide chain with two or more, preferably 2, 3, 4, 5, 6, 7, 8, 9 or 10 nucleosides linked by phosphate and/or amid links, i.e. are RNA, DNA or PNA chains or mixtures thereof, preferably DNA or RNA. The use of a polynucleotide rather than a mononucleotide can improve the speed of the reaction, however, the length of the polynucleotide should generally not exceed 10 nucleotides or otherwise the specificity of the coupling step will depend less on the nucleotide at the terminus, i.e. the one that is coupled to the polynucleotide primer, but rather on the interaction of the flanking nucleotides.

In a further embodiment of the method of the present invention the activating reagent is a substance with a structure according to formula (XL)

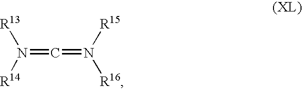

wherein
R¹³ and R¹⁶ independent of each other mean H; linear or branched, substituted or unsubstituted C₁ to C₁₀ alkyl, e.g. C₁, C₂, C₃, C₄, C₅, C₆, C₇, C₈, C₉ or C₁₀, in particular methyl, ethyl, propyl, butyl, iso-butyl, tert-butyl, linear or branched C₁ to C₁₀ alkyl-NR¹⁷R¹⁸, e.g. C₁, C₂, C₃, C₄, C₅, C₆, C₇, C₈, C₉ or C₁₀, in particular methyl, ethyl, propyl, butyl, iso-butyl, tert-butyl, wherein R¹⁷ and R¹⁸ independent of each other mean H, linear or branched substituted or unsubstituted C₁ to C₅ alkyl, e.g. C₁, C₂, C₃, C₄ or C₈, independent of each other mean methyl, ethyl, propyl, butyl, iso-butyl, tert-butyl; C₃ to C₈ cycloalkyl, e.g. cylcopropyl, cyclobutyl, cyclopentyl, cyclohexyl, cycloheptyl, cyclooctyl, aryl, e.g. phenyl or heteroaryl;
R¹⁴ and R¹⁵ either mean a free electron pair or R¹³ and R¹⁴ and/or R¹⁵ and R¹⁶ together form a heteroaryl, in particular pyridyl;
or are 2-fluoro pyridine; R^{V}—CO—Cl; or Z-SO₂—R^{V}, wherein R^{V} has the meaning saturated or unsaturated, C₁ to C₁₀ alkyl, e.g. C₁, C₂, C₃, C₄, C₅, C₆, C₇, C₈, C₉ or C₁₀ alkyl, in particular methyl, ethyl, n-propyl, iso-propyl, n-butyl, tert-butyl, or pentyl, C₁, C₂, C₃, C₄, C₅, C₆, C₇, C₈, C₉ or C₁₀ alkenyl, in particular ethenyl, 1-propenyl, 2-propenyl, butenyl, or pentenyl, C₁, C₂, C₃, C₄, C₅, C₆, C₇, C₈, C₉ or C₁₀ alkinyl, in particular ethinyl, 1-propinyl, 3-propinyl, butinyl, or pentinyl; aryl; heteroaryl, which can be substituted with one or more OH, SH, NH₂, F, Cl, Br, or I
and the activation of the nucleotide or polynucleotide is carried out in the presence of a catalyst selected from the group consisting of a structure according to formula (XLI) to (L)

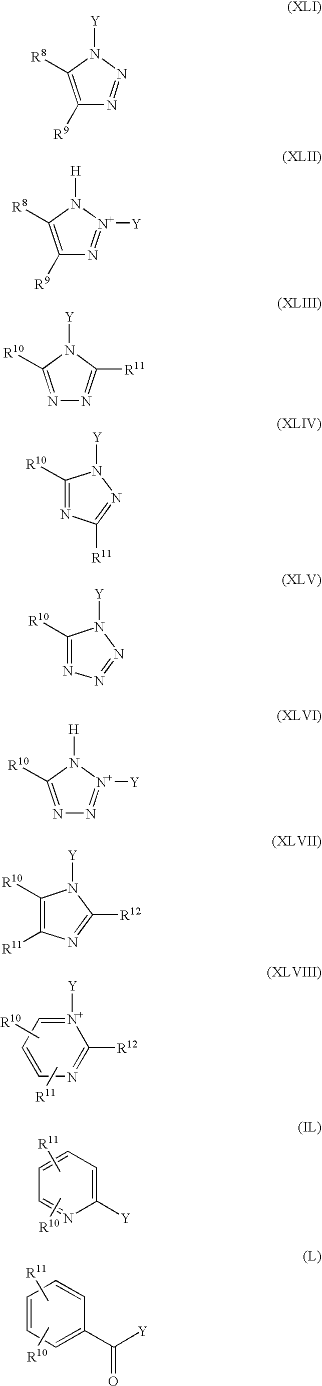

wherein R⁸ and R⁹ independent of each other have the meaning H; OH; SH; NH₂; F; Cl; Br; I; saturated or unsaturated, linear or branched, unsubstituted or substituted C₁ to C₁₀ alkyl, e.g. C₁, C₂, C₃, C₄, C₅, C₆, C₇, C₈, C₉ or C₁₀ alkyl, in particular methyl, ethyl, n-propyl, iso-propyl, n-butyl, tert-butyl, or pentyl, C₁, C₂, C₃, C₄, C₅, C₆, C₇, C₈, C₉ or C₁₀ alkenyl, in particular ethenyl, 1-propenyl, 2-propenyl, butenyl, or pentenyl, C₁, C₂, C₃, C₄, C₅, C₆, C₇, C₈, C₉ or C₁₀ alkinyl, ethinyl, 1-propinyl, 3-propinyl, butinyl, or pentinyl; or taken together form a saturated or unsaturated, unsubstituted or substituted mono, bi or polycyclic ring, in particular an aryl or heteroaryl substituted with one, two three, or four substituents selected from the group consisting of Cl and F;
R¹⁰ and R¹¹ independent of each other have the meaning H; OH; SH; NH₂; F; Cl; Br; 1; saturated or unsaturated, linear or branched, unsubstituted or substituted C₁ to C₁₀ alkyl, e.g. C₁, C₂, C₃, C₄, C₅, C₆, C₇, C₈, C₉ or C₁₀ alkyl, in particular methyl, ethyl, n-propyl, iso-propyl, n-butyl, tert-butyl, or pentyl, C₁, C₂, C₃, C₄, C₅, C₆, C₇, C₈, C₉ or C₁₀ alkenyl, in particular ethenyl, 1-propenyl, 2-propenyl, butenyl, or pentenyl, C₁, C₂, C₃, C₄, C₅, C₆, C₇, C₈, C₉ or C₁₀ alkinyl, in particular ethinyl, 1-propinyl, 3-propinyl, butinyl, or pentinyl, linear or branched C₁ to C₁₀ alkyl-NR¹⁹R²⁰, e.g. C₁, C₂, C₃, C₄, C₅, C₆, C₇, C₈, C₉ or C₁₀, in particular methyl, ethyl, propyl, butyl, iso-butyl, tert-butyl wherein R¹⁹ and R²⁰ independent of each other mean linear or branched substituted or unsubstituted C₁ to C₁₀ alkyl, e.g. C₁, C₂, C₃, C₄, C₅, C₆, C₇, C₈, C₉ or C₁₀, in particular methyl, ethyl, propyl, butyl, iso-butyl, tert-butyl; C₃ to C₉ cycloalkyl, cyclopropyl, cyclobutyl, cyclopentyl, cyclohexyl, cycloheptyl, cyclooctyl; aryl; or heteroaryl;
R¹² has the meaning H, OH, SH, NH₂, F, Cl, Br, I, CH₃, substituted methyl, saturated or unsaturated, linear or branched, unsubstituted or substituted C₂ to C₅ alkyl, C₂ to C₅ alkyl, e.g. C₂, C₃, C₄ or C₅ alkyl, in particular, methyl, ethyl, n-propyl, iso-propyl, n-butyl, tert-butyl, pentyl, C₂, C₃, C₄ or C₅ alkenyl, in particular ethenyl, 1-propenyl, 2-propenyl, butenyl, pentenyl, C₂, C₃; C₄ or C₅ alkinyl, in particular, ethinyl, 1-propinyl, 2-propinyl butinyl or pentinyl,
and Y is selected from the group consisting of H and OH.

The term “aryl” as used above preferably refers to an aromatic monocyclic ring containing 6 carbon atoms, an aromatic bicyclic ring system containing 10 carbon atoms or an aromatic tricyclic ring system containing 14 carbon atoms. Examples are phenyl, naphtalenyl or anthracenyl. The aryl group is optionally substituted. The term “heteroaryl” preferably refers to a five or six-membered aromatic monocyclic ring wherein at least one of the carbon atoms are replaced by 1, 2, 3, or 4 (for the five membered ring) or 1, 2, 3, 4, or 5 (for the six membered ring) of the same or different heteroatoms, preferably selected from O, N and S; an aromatic bicyclic ring system wherein 1, 2, 3, 4, 5, or 6 carbon atoms of the 8, 9, 10, 11 or 12 carbon atoms have been replaced with the same or different heteroatoms, preferably selected from O, N and S; or an aromatic tricyclic ring system wherein 1, 2, 3, 4, 5, or 6 carbon atoms of the 13, 14, 15, or 16 carbon atoms have been replaced with the same or different heteroatoms, preferably selected from O, N and S. Examples are oxazolyl, isoxazolyl, 1,2,5-oxadiazolyl, 1,2,3-oxadiazolyl, pyrrolyl, imidazolyl, pyrazolyl, 1,2,3-triazolyl, thiazolyl, isothiazolyl, 1,2,3,-thiadiazolyl, 1,2,5-thiadiazolyl, pyridinyl, pyrimidinyl, pyrazinyl, 1,2,3-triazinyl, 1,2,4-triazinyl, 1,3,5-triazinyl, 1-benzofuranyl, 2-benzofuranyl, indoyl, isoindoyl, benzothiophenyl, 2-benzothiophenyl, 1H-indazolyl, benzimidazolyl, benzoxazolyl, indoxazinyl, 2,1-benzosoxazoyl, benzothiazolyl, 1,2-benzisothiazolyl, 2,1-benzisothiazolyl, benzotriazolyl, quinolinyl, isoquinolinyl, quinoxalinyl, quinazolinyl, quinolinyl, 1,2,3-benzotriazinyl, or 1,2,4-benzotriazinyl.

If R⁸ and R⁹ are taken together to form a saturated or unsaturated mono, bi or polycyclic ring system in the context of the five-membered heteroaryls according to (II) to (V), (X) and (XIV) they preferably form a cyclopentadienyl, benzyl, napthyl, pyridinyl, pyrimidinyl, pyrazinyl, 1,2,3-triazinyl, 1,2,4-triazinyl and bicyclo[2.2.1]hepta-3-en.

If R⁸ and R⁹ are taken together to form a saturated or unsatured mono, bi or polyclyclic ring system in the context of the six-membered aryls or heteroaryls according to (X) to (XIII), (XV) to (XIX) furanyl, oxazolyl, isoxazolyl, 1,2,5-oxadiazolyl, 1,2,3-oxadiazolyl; pyrrolyl, imidazolyl, pyrazolyl, 1,2,3-triazolyl, thiazolyl, isothiazolyl, 1,2,3,-thiadiazolyl, 1,2,5-thiadiazolyl, or thiophenyl, pyridinyl, pyrimidinyl, pyrazinyl, 1,2,3-triazinyl; 1,2,4-triazinyl.

The coupling reactions of the present invention can be carried out in a variety of solvents including aqueous solutions. Preferably aqueous solutions further comprise butters, like, e.g. tris-HCl, HEPES, PIPES and the like and salts including e.g. NaCl, KCl and the like.

In a preferred embodiment of the method of the present invention the catalyst is selected from the group consisting of imidazole, methylimidazole, benzimidazole, triazole, tetrazole, hydroxybenzotriazole, azahydroxybenzotriazole, chlorobenzotriazole, dimethylaminopyridine (DMP).

Preferably the side chains R¹³ and R¹⁶ of the activating reagent mean CH₃, C₂H₅, C₃H₇, C(CH₃)₃, C₂H₄N(CH₃)₂, cycloC₆H₁₁ and C₃H₆N(CH₃)₂. It is even more preferred that the activating agent used in conjunction with a catalyst is selected from the group consisting of N-(3-dimethylaminopropyl)-N′-ethylcarbodiimid (EDC), N,N′-diisopropylcarbodiimide (DIC), and N,N′-dicyclohhexylcarbodiimide (DCC), N,N′-carbonyl diimidazole (CDI), t-butyl-ethylcarbodiimide and t-butyl-methylcarbodiimide.

As outlined above with respect to the nucleotide of the present invention it is in some embodiments preferred that the nucleotide or polynucleotide comprises a stacking residue in order to increase the interaction with the polynucleotide template, thus, in one embodiment of the method of the present invention the nucleotide or the polynucleotide further comprises a stacking residue. The term “stacking residue” has the meaning and preferred meaning as outlined above. Particular preferred stacking residues are selected from the group consisting of indole, napthol, a steroid ring system, bile acid, quinoline, quinolone, stilbene, pyrene, anthraquinone, an ethidium residue, an anthracene residue, and tetracene, which can be substituted with one or more residues selected from the group consisting of OH, SH, NH₂, F, Cl, Br and I.

Alternatively or additionally the nucleotide or polynucleotide can comprise a marker residue. Again this term is used as outlined above, thus, the marker residue can be any residue, which facilitates detection of the reaction product in an assay system. However, preferred markers are selected from a fluorescent residue, a radioactive residue, a phosphorescent residue, a chelating residue comprising a metal ion and a quenching residue.

Although, it is possible to couple the terminal amino group of a polynucleotide primer located at any position, e.g. at the 2′, 3′ or 5′ position, to an activated phosphate group or carboxy group of the nucleotide or polynucleotide at any position, e.g. at the 2′, 3′ and 5′ position, it is preferred that a polynucleotide primer with one 2′ or 3′ terminal amino group is reacted with an nucleotide of the present invention, comprising a 5′ terminal activated phosphate ester or carboxylic ester, or with a nucleotide or a polynucleotide with a 5′ terminal activated phosphate or carboxy residue. This mode of coupling leads to an extension of the polynucleotide primer at 2′ or 3′ end and, thus, allows to determine the sequence of a polynucleotide template 3′ of the polynucleotide primer. Alternatively, a polynucleotide primer with a 5′ terminal amino group is reacted with a nucleotide of the present invention, comprising one 2′ or 3′ terminal activated phosphate ester or carboxylic ester, or with a nucleotide or a polynucleotide with one 2′ or 3′ terminal activated phosphate or carboxy residue. This mode of coupling will allow the analysis of template sequences 5′ of the polynucleotide primer. Both modes of coupling are particularly preferred in the context of template directed coupling reactions.

In most cases the coupling reaction will be carried out in a template directed manner, i.e. the identity of the coupled nucleotide or polynucleotide will be determined by the base sequence of a template strand. Thus, in a particular preferred embodiment the method of the present invention comprises the further step of annealing the polynucleotide primer to a single or double stranded polynucleotide template.

The term “polynucleotide template” as used herein refers to a nucleotide chain with two or more, preferably 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 550, 600, 650, 700, 750, 800, 850, 900, 950, 1000 or up to several million (size of a chromosome) nucleosides linked by phosphate and/or amid links, i.e. are RNA, DNA or PNA chains or mixtures thereof, preferably DNA or RNA chains. The polynucleotide template employed in the method of the present invention can have any length. It can be of natural or synthetic origin. It can be single, double or triple stranded. It can be derived from any biological source, e.g. genomic DNA, plasmid DNA, viral DNA or RNA, mRNA, tRNA, rRNA, snRNA, mitochondrial DNA, or it can be the product of an amplification reaction, e.g. PCR. If the polynucleotide template is double stranded or is in the form of a triple helix, it needs denatured to be at least partially single stranded within the region to which the polynucleotide primer anneals. Usually this is achieved by chemical, e.g. by alkaline treatment, or heat denaturation, e.g. boiling, of a nucleic acid double or triple strand and subsequent chemical treatment, e.g. neutralization, or cooling to anneal the completely or partially single stranded polynucleotide template to the polynucleotide primer. Thus, in a typical application of the extension method of the present invention the DNA or RNA probe, e.g. genomic DNA or a PCR amplified product, to be analyzed is denatured, brought into contact with the polynucleotide primer (alternatively the polynucleotide primer is already present during the denaturation step), annealed and subsequently extended. To facilitate extension of the polynucleotide primer, i.e. the addition of a nucleotide of the invention or of an activated nucleotide or polynucleotide, that is capable of specific base pairing with the template it is preferred that the polynucleotide template comprises at least a one nucleotide overhang 2′, 3′ and/or 5′ with respect to the polynucleotide primer.

It has been discovered that the kinetics of the extension reaction are further enhanced, if the overhang of the polynucleotide template is larger than just one nucleotide, thus, in a preferred embodiment of the method of the present invention the overhang has a length of four or more nucleotides, e.g. 4, 5, 6, 7, 8, 9, 10, 11, 12, 13, 14, 15 or more nucleotides.

Surprisingly, it has been found that the rate of the reaction can be further enhanced, if a further polynucleotide termed “polynucleotide helper” is annealed to the polynucleotide template. The observed increase in the reaction speed with a polynucleotide helper is at least 4-fold. Thus, in a preferred embodiment of method of the present invention the method comprises the further step of annealing a polynucleotide helper or a polynucleotide helper comprising a stacking residue to the polynucleotide template. This annealing step can be carried out between the polynucleotide template and the polynucleotide helper prior to annealing to the polynucleotide primer or alternatively all three polynucleotides can be annealed concomitantly or the polynucleotide helper can be annealed after annealing of the two other polynucleotides.

The term “polynucleotide helper” as used herein refers to a nucleotide chain with two or more, preferably 2, 3, 4, 5, 6, 7, 8, 9, 10, 11, 12, 13, 14, 15, 16, 17, 18, 19, 20, or more nucleosides linked by phosphate and/or amid links, i.e. is a RNA, DNA or PNA chain or a mixture thereof, preferably a DNA or RNA chain. Polynucleotide helpers used in a method of the present invention may have a length of between 2 and 400 bp, preferably between 4 and 100 bp and more preferably between 8 and 20 bp.

The polynucleotide helper employed in the reaction can be in solution or can be linked directly or indirectly to a surface. If the polynucleotide helper is linked to a surface than it is preferred that the polynucleotide template and polynucleotide primer are provided in solution and are “captured” on the surface by the polynucleotide helper. Suitable surfaces are without limitation glass, metal, e.g. gold, plastic, e.g. Teflon®, polystyrol, polypropylene, polyethylene, polycarbonate, silizium oxide, and the like. The surface can have any three-dimensional shape. It can be flat or can be on a bead, e.g. SiO₂ or rubber coated magnetic bead, and can take on any shape suitable to allow the extension reaction to take place. If the surface is part of a chip it can additionally have inlet and outlet ports, flow lines, waste and buffer compartments, reaction chambers, e.g. DNA purification or PCR amplification chambers, as required and known in the art. Accordingly, the method of the present invention can also be carried out on a chip coated with one or more polynucleotide helpers This chip can be packaged in a kit, which can optionally include one or more nucleotides of the present invention or activating reagents and optionally nucleotides or polynucleotides with an activatable phosphate or carboxy residue. The indirect link of the polynucleotide helper can be through a polynucleotide capture probe. Accordingly, the chip may also comprise polynucleotide capture probes and optionally polynucleotide helpers, which can be “captured” by the capture probes.

It has been found by the present inventors that the effect of the polynucleotide helper can be even more enhanced, if the polynucleotide helper comprises a stacking residue. The stacking residue can be a substituted or unsubstituted homo or heteroaryl ring system preferably with two, three or four rings, with a size similar to a G-C or A-T base pair. The stacking residue is preferably selected from the group consisting of substituted or unsubstituted indole, napthol, a steroid ring system, bile acid, quinoline, quinolone, stilbene, pyrene, anthraquinone, an ethidium residue, an anthracene residue, and tetracene, which can be substituted with one or more residues selected from the group consisting of OH, SH, NH₂, F, Cl, Br and I.

If the polynucleotide helper is used in the method of the present invention it is preferred that the length of the nucleotide gap between the annealed polynucleotide helper or a polynucleotide helper comprising a stacking residue and the annealed polynucleotide primer is identical to the length of the nucleotide of the present invention, comprising a terminal activated phosphor ester or carboxylic ester, or the length of the nucleotide or the polynucleotide comprising an activated terminal phosphate or carboxy residue, which is coupled to the polynucleotide primer.

In certain embodiments of the method employing a polynucleotide helper with a stacking residue it is preferred that the length of the nucleotide gap between the annealed polynucleotide helper comprising a stacking residue and the polynucleotide primer is one nucleotide larger than the length of the nucleotide of the present invention, comprising a terminal activated phosphor ester or carboxylic ester, or the length of the nucleotide or the polynucleotide comprising an activated terminal phosphate or carboxy residue, which is coupled to the polynucleotide primer. Preferably, in this embodiment the stacking residue is attached at the nucleotide directly adjacent to the gap between the polynucleotide helper and polynucleotide primer. In this embodiment it is hypothized that the stacking residue interacts with the base adjacent to the base with which the nucleotide interacts and thus, facilitates coupling of the nucleotide.

It is possible that only one type of nucleotide of the present invention or only one nucleotide or polynucleotide comprising an activated terminal phosphate or carboxy residue is present in the coupling reaction. However, due to the high specificity of the template directed extension reaction of the present invention it is also possible to provide two, three, four or more different nucleotides in one coupling reaction out of which, e.g. only one nucleotide will be coupled to the polynucleotide primer due to base specific interaction with the template. Thus, in a preferred embodiment of the method of the present invention at least two nucleotides carrying different bases are included in step b). For example, when analysing the methylation status of a genomic cytosine the bases of the two different nucleotides would need to be able to specifically pair with C or T in the template or alternatively, if the other strand is analyzed with G or A. In such a process the coupling of a nucleotide capable of base pairing with G or C would be indicative of 5-methylation of the cytosine in the underlying genomic sequence, while A or T would be indicative of a lack of methylation.

Typically, the coupling reaction is only carried out once, i.e. one nucleotide or polynucleotide is added in a sequence specific manner, however, it is envisioned that the coupling reaction is carried out two or more times to generate longer extension products and/or to determine the sequence of consecutive base pairs on the polynucleotide template. In a preferred embodiment of the method of the present invention the step of coupling the nucleotide of the invention or a nucleotide or polynucleotide comprising an activated phosphate or carboxy residue to the polynucleotide primer is repeated one or more times, e.g. 2, 3, 4, 5, 6, 7, 8, 9, 10 to 1000 times or more. A precondition to such a repetition of coupling steps is that the nucleotide or polynucleotide added comprises a free 2′, 3′ or 5′ terminal amino group or can be rendered to comprise such a group. This 2′, 3′ or 5′ terminal amino group will then react with a further nucleotide or polynucleotide. Multiple rounds of coupling are particularly preferred, if the polynucleotide primer is immobilized and so called “on-chip-sequencing” is performed. If two or more coupling reactions are carried out, it is preferred that the respective nucleotide added comprise an amino or carboxy terminus protected with a photo cleavable protection group to which a marker, preferably a fluorescent marker is attached.

In one preferred embodiment of the method of the present invention the method further comprises the step of photo cleavage of the spacer. This preferably leads to the release of a fluorescent dye and exposes a free amino or carboxy terminus capable of reacting with a further nucleotide of the invention, which may carry a further marker, preferably fluorescent marker.

Since one of the primary aims of the method of the present invention is the determination of the sequence of the polynucleotide template the method of the present invention further comprises the step of analyzing the reaction product of step b). Based on the known pairing rules of bases such an analysis allows the determination of the sequence of one nucleotide or in case that a polynucleotide was coupled of a few nucleotides 3′ or 5′ to the polynucleotide primer. Numerous methods for analysing the extension product are known in the art, however, in a preferred embodiment the analysis is carried out by mass spectrometry, mass sensing, radiometry, fluorescence spectroscopy or phosphorescence spectroscopy, electrophoresis, chromatography, or atomic force microscopy. If the coupling reaction is carried out two or more times each coupling reaction can be followed by analysis step.

As has been set out above the art known methods for non-enzymatic extension of polynucleotides were all to slow and unspecific to allow template directed extension of a polynucleotide primer. However, the nucleotides and methods of the present invention improve both specificity and speed of the coupling reaction to such an extent that the analysis of the sequence of a polynucleotide, preferentially of a DNA or RNA can be attempted. Consequently, the present invention is in a further aspect directed at the use of a template directed non-enzymatic extension of a polynucleotide for the determination of the sequence of a polynucleotide template 5′ or 3′-terminal from an annealed polynucleotide primer.

In a typical application of the nucleotide and/or the methods of the present invention only one coupling reaction is carried out, therefore, unless a polynucleotide is used for coupling the use will only allow the determination of on base on the 3′ or 5′ terminal side of the polynucleotide primer. The determination of single bases is particularly important in the context of analysing SNPs, point mutations, chromosomal rearrangements, base modifications, in particular cytosine methylations, splice variants, deletions or loss of nucleobases. For these uses the polynucleotide primer is preferably chosen to anneal directly adjacent to the potential mutated, modified or rearranged sequence and the extension of the primer by only one nucleotide will allow a conclusion on whether a given SNP, modification or rearrangement is present or not in a given probe. For other applications including, for example, “on-chip sequencing” several rounds of coupling might be required.

It is preferred that a nucleotide of the present invention or a method of the present invention is used for the determination of the sequence of a polynucleotide template 5′ or 3′-terminal from an annealed polynucleotide primer. Again the determination of SNPs, point mutations, chromosomal rearrangements, base modifications, in particular cytosine methylations, splice variants, deletions or loss of nucleobases is preferred.

Due to the significant increase in reaction speed it is preferred that a polynucleotide helper with or without a stacking residue is used.

As pointed out above the coupling reaction step of the methods of the present invention can take place in solution or it is possible to directly or indirectly, e.g. via a polynucleotide capture probe, the polynucleotide primer or the polynucleotide helper, if used to a surface. Alternatively the polynucleotide template can directly or indirectly linked to a surface, e.g. a chip. Again such immobilisation can be through a capture probe.

A further aspect of the present invention is a kit comprising at least one nucleotide of the present invention and a polynucleotide primer, with at least one 2′, 3′ or 5′terminal amino group.

A further aspect of the present invention is a fit comprising at least one activating reagent and a nucleotide or polynucleotide comprising an activatable phosphate or carboxy residue. Preferably the nucleotides or polynucleotides carry a single terminal phosphate residue, i.e. are not nucleotide triphosphates but rather monophosphates. In this context any of the activating reagents indicated above can be included in the kit, e.g. pentafluorophenyl ester reagents, phosphonium reagents, uronium reagents, or an acid fluoride reagents. Even more suitable activating reagents are selected from the group comprising ACTU, HATU, HBTU, HCTU, HAPyU, HBPyU, HCPyU, TBTU, TCTU, TNTU, TPTU, HSTU, TSTU, PFTU, TFFH, TCFH, BTFFH, TOTU, FDPP, PfPyU, PffU, AOP, BOP, COP, PyAOP, PyBOP, and PyCOP. Particular preferred activating reagents are HATU, HBTU and HCTU. In a preferred embodiment this kit further comprises a polynucleotide primer, with at least one 2′, 3′ or 5′terminal amino group.

The polynucleotide primer can be immobilized on a surface, e.g. a chip surface. The kit can, for example, comprise a chip with 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000, 1500, 2000, 2500, 3000 or more polynucleotide primers, preferably in separate areas. Upon hybridization of such a chip to a biological sample it is possible to simultaneously extend all polynucleotide primers and, thus, determine the sequence of any given number of gene sequences simultaneously.

Another aspect of the present invention is a surface to which one or more polynucleotide primers, i.e. comprising a 2′, 3′ or 5′ terminal amino group, are coupled. Preferably, such a surface comprises between 2 and 1,000,000 polynucleotide primers, e.g. 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, 300, 350, 400, 450, 500 or more polynucleotide primers in separate areas.

A further preferred activating reagent, which can be included in the kit has a structure according to formula (XXXIV)

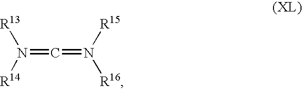

wherein
R″ and R¹⁶ independent of each other mean H; linear or branched, substituted or unsubstituted C₁ to C₁₀ alkyl, e.g. C₁, C₂, C₃, C₄, C₅, C₆, C₇, C₈, C₉ or C₁₀, in particular methyl, ethyl, propyl, butyl, iso-butyl, tert-butyl, linear or branched C₁ to C₁₀ alkyl-NR¹⁷R¹⁸, e.g. C₁, C₂, C₃, C₄, C₅, C₆, C₇, C₈, C₉ or C₁₀, in particular methyl, ethyl, propyl, butyl, iso-butyl, tert-butyl, wherein R¹⁷ and R¹⁸ independent of each other mean H, linear or branched substituted or unsubstituted C₁ to C₅ alkyl, e.g. C₁, C₂, C₃, C₄ or C₅, independent of each other mean methyl, ethyl, propyl, butyl, iso-butyl, tert-butyl; C₃ to C₈ cycloalkyl, e.g. cylcopropyl, cyclobutyl, cyclopentyl, cyclohexyl, cycloheptyl, cyclooctyl; aryl; e.g. phenyl; or heteroaryl;
R¹⁴ and R¹⁵ either mean a free electron pair or R¹³ and R¹⁴ and/or R¹⁵ and R¹⁶ together form a heteroaryl in particular pyridyl.

A further preferred activating reagent is selected from 2-fluoro pyridine; R^{V}—CO—Cl; or Z-SO₂—R^{V}, wherein R^{V} has the meaning saturated or unsaturated, C₁ to C₁₀ alkyl, e.g. C₁, C₂, C₃, C₄, C₅, C₆, C₇, C₈, C₉ or C₁₀ alkyl, in particular methyl, ethyl, n-propyl, iso-propyl, n-butyl, tert-butyl, or pentyl, C₁, C₂, C₃, C₄, C₅, C₆, C₇, C₈, C₉ or C₁₀ alkenyl, in particular ethenyl, 1-propenyl, 2-propenyl, butenyl, or pentenyl, C₁, C₂, C₃, C₄, C₅, C₆, C₇, C₈, C₉ or C₁₀ alkinyl, in particular ethinyl, 1-propinyl, 3-propinyl, butinyl, or pentinyl, aryl, heteroaryl, which can be substituted with one or more OH, SH, NH₂, F, Cl, Br, or I.

Preferably, this kit further comprises at least one catalyst with a structure according to formulas (XXXV) to (XXXIV)

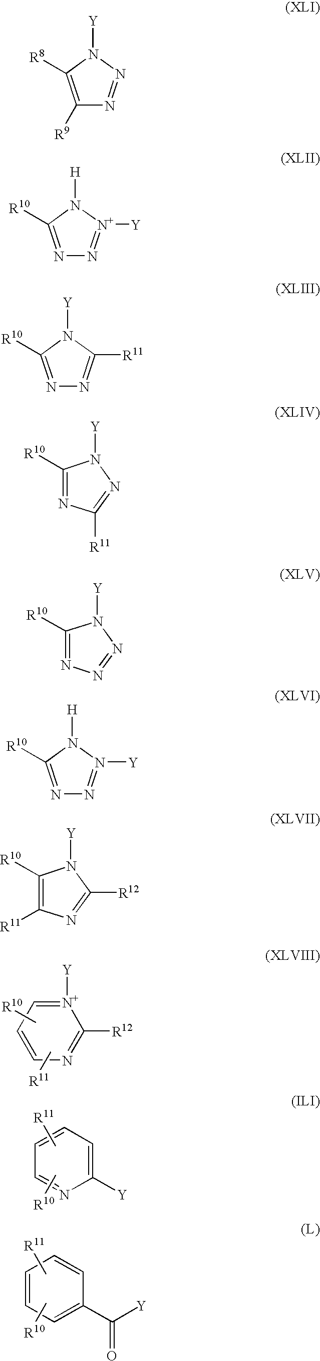

wherein R⁸ and R⁹ independent of each other have the meaning H; OH; SH; NH₂; F; Cl; Br; I; saturated or unsaturated, linear or branched, unsubstituted or substituted C₁ to C₁₀ alkyl, e.g. C₁, C₂, C₃, C₄, C₅, C₆; C₇, C₈, C₉ or C₁₀ alkyl, in particular methyl, ethyl, n-propyl, iso-propyl, n-butyl, tert-butyl, or pentyl, C₁, C₂, C₃, C₄, C₅, C₆, C₇, C₈, C₉ or C₁₀ alkinyl, in particular ethenyl, 1-propenyl, 2-propenyl, butenyl, or pentenyl, C₁, C₂, C₃, C₄, C₅, C₆, C₇, C₈, C₉ or C₁₀ alkinyl, in particular ethinyl, 1-propinyl, 3-propinyl, butinyl, or pentinyl; or taken together form a saturated or unsaturated, unsubstituted or substituted mono, bi or polycyclic ring, in particular an aryl or heteroaryl substituted with one, two three, or four substituents selected from the group consisting of Cl and F;
R¹⁰ and R¹¹ independent of each other have the meaning H; OH; SH; NH₂; F; Cl; Br; I; saturated or unsaturated, linear or branched, unsubstituted or substituted C₁ to C₁₀ alkyl, e.g. C₁, C₂, C₃, C₄, C₅, C₆, C₇, C₈, C₉ or C₁₀ alkyl, in particular methyl, ethyl, n-propyl, iso-propyl, n-butyl, tert-butyl, or pentyl, C₁, C₂, C₃, C₄, C₅, C₆, C₇, C₈, C₉ or C₁₀ alkenyl, in particular ethenyl, 1-propenyl, 2-propenyl, butenyl, or pentenyl, C₁, C₂, C₃, C₄, C₅, C₆, C₇, C₈, C₉ or C₁₀ alkinyl, in particular ethinyl, 1-propinyl, 3-propinyl, butinyl, or pentinyl; linear or branched C₁ to C₁₀ alkyl-NR¹⁹R²⁰, e.g. C₁, C₂, C₃, C₄, C₅, C₆, C₇, C₈, C₉ or C₁₀, in particular methyl, ethyl, propyl, butyl, iso-butyl, tert-butyl, wherein R¹⁹ and R²⁰ independent of each other mean linear or branched substituted or unsubstituted C₁ to C₁₀ alkyl, e.g. C₁, C₂, C₃, C₄, C₅, C₆, C₇, C₈, C₉ or C₁₀, in particular methyl, ethyl, propyl, butyl, iso-butyl, tert-butyl, C₃ to C₉ cycloalkyl, cyclopropyl, cyclobutyl, cyclopentyl, cyclohexyl, cycloheptyl, cyclooctyl, aryl, or heteroaryl;
R¹² has the meaning H; OH; SH; NH₂; F; Cl; Br; I; CH₃; substituted methyl, saturated or unsaturated, linear or branched, unsubstituted or substituted C₂ to C₅ alkyl, C₂ to C₅ alkyl, e.g. C₂, C₃, C₄ or C₅ alkyl, in particular methyl, ethyl, n-propyl, iso-propyl, n-butyl, tert-butyl, pentyl, C₂, C₃, C₄ or C₅ alkenyl, in particular ethenyl, 1-propenyl, 2-propenyl, butenyl, pentenyl, C₂, C₃; C₄ or C₅ alkinyl, in particular ethinyl, 1-propinyl, 2-propinyl butinyl or pentinyl,
and Y is selected from the group consisting of H and OH.

The term “aryl” as used above preferably refers to an aromatic monocyclic ring containing 6 carbon atoms, an aromatic bicyclic ring system containing 10 carbon atoms or an aromatic tricyclic ring system containing 14 carbon atoms. Examples are phenyl, naphtalenyl or anthracenyl. The aryl group is optionally substituted. The term “heteroaryl” preferably refers to a five or six-membered aromatic monocyclic ring wherein at least one of the carbon atoms are replaced by 1, 2, 3, or 4 (for the five membered ring) or 1, 2, 3, 4, or 5 (for the six membered ring) of the same or different heteroatoms, preferably selected from O, N and S; an aromatic bicyclic ring system wherein 1, 2, 3, 4, 5, or 6 carbon atoms of the 8, 9, 10, 11 or 12 carbon atoms have been replaced with the same or different heteroatoms, preferably selected from O, N and S; or an aromatic tricyclic ring system wherein 1, 2, 3, 4, 5, or 6 carbon atoms of the 13, 14, 15, or 16 carbon atoms have been replaced with the same or different heteroatoms, preferably selected from O, N and S. Examples are oxazolyl, isoxazolyl, 1,2,5-oxadiazolyl, 1,2,3-oxadiazolyl, pyrrolyl, imidazolyl, pyrazolyl, 1,2,3-triazolyl, thiazolyl, isothiazolyl, 1,2,3,-thiadiazolyl, 1,2,5-thiadiazolyl, pyridinyl, pyrimidinyl, pyrazinyl, 1,2,3-triazinyl, 1,2,4-triazinyl, 1,3,5-triazinyl, 1-benzofuranyl, 2-benzofuranyl, indoyl, isoindoyl, benzothiophenyl, 2-benzothiophenyl, 1H-indazolyl, benzimidazolyl, benzoxazolyl, indoxazinyl, 2,1-benzosoxazoyl, benzothiazolyl, 1,2-benzisothiazolyl, 2,1-benzisothiazolyl, benzotriazolyl, quinolinyl, isoquinolinyl, quinoxalinyl, quinazolinyl, quinolinyl, 1,2,3-benzotriazinyl, or 1,2,4-benzotriazinyl.

If R⁸ and R⁹ are taken together to form a saturated or unsaturated mono, bi or polycyclic ring system in the context of the five-membered heteroaryls according to (II) to (V), (X) and (XIV) they preferably form a cyclopentadienyl, benzyl, napthyl, pyridinyl, pyrimidinyl, pyrazinyl, 1,2,3-thiazinyl, 1,2,4-triazinyl, and bicyclo[2.2.1]hepta-3-en.

If R⁸ and R⁹ are taken together to form a saturated or unsatured mono, bi or polyclyclic ring system in the context of the six-membered aryls or heteroaryls according to (X) to (XIII), (XV) to (XIX) furanyl, oxazolyl, isoxazolyl, 1,2,5-oxadiazolyl, 1,2,3-oxadiazolyl; pyrrolyl, imidazolyl, pyrazolyl, 1,2,3-triazolyl, thiazolyl, isothiazolyl, 1,2,3,-thiadiazolyl, 1,2,5-thiadiazolyl, or thiophenyl, pyridinyl, pyrimidinyl, pyrazinyl, 1,2,3-triazinyl; 1,2,4-triazinyl.

In a preferred embodiment the kit of the present invention further comprises a polynucleotide helper or a polynucleotide helper comprising a stacking residue.

The following examples are included to demonstrate preferred embodiments of the invention. It should be appreciated by those of skill in the art that the techniques disclosed in the examples that follow represent techniques discovered by the inventors to function well in the practice of the invention, and thus, can be considered preferred modes for its practise. However, those of skill in the art should, in light of the present disclosure, appreciate that many changes can be made in the specific embodiments that are disclosed without departing from the spirit and scope of the invention as set out in the appended claims. All references cited are incorporated herein by reference.

### DESCRIPTION OF FIGURES AND TABLES

FIG. 1 Schematic representation of the conventional determination of base sequence by polymerase mediated primer extension reaction. Depending on which of the four core bases (B′) is present in the template strand at the position corresponding to the position where the new base is to be added one of the four possible nucleotide triphosphates, i.e. the one with the complementary core base, is added to the primer. The reaction is catalyzed in the reactive centre of the polymerase. In case of dideoxychain termination sequencing R has the meaning H.

FIG. 2 Schematic representation of the components of a non enzymatic primer extension reaction involving a polynucleotide helper.

FIG. 3 Schematic representation of a non enzymatic primer extension reaction with a polynucleotide helper.

FIG. 4 Schematic representation of a non enzymatic primer extension reaction with a polynucleotide helper comprising a stacking residue.

FIG. 5 Schematic representation of a non enzymatic primer extension reaction using RNA polynucleotides including an RNA polynucleotide helper.

FIG. 6 shows MALDI-TOF mass spectra of extension reactions after 30 min and 3 h using a Cy3 labelled HOAt-CMP.

FIG. 7 (A) to (D) show MALDI-TOF mass spectra of extension reactions after 20 min with 36 picomol/μ template/primer using four different templates and the nucleotides, T, A, G and C, respectively.

FIG. 8 shows MALDI-TOF mass spectra of extension reactions after 4 h at 20° C. using different dCMP derivatives.

FIG. 9 shows MALDI-TOF mass spectra of extension reactions after 16 h at 20° C. using different dGMP and different catalysts.

FIG. 10 Example of a non-enzymatic primer extension reaction on a gold surface on a microchip.

FIG. 11 Example of a sequencing method of the present invention using photolabile fluorophores in the coupling reactions. PC stands for photolabile linker, dye is preferably a fluorescent dye.

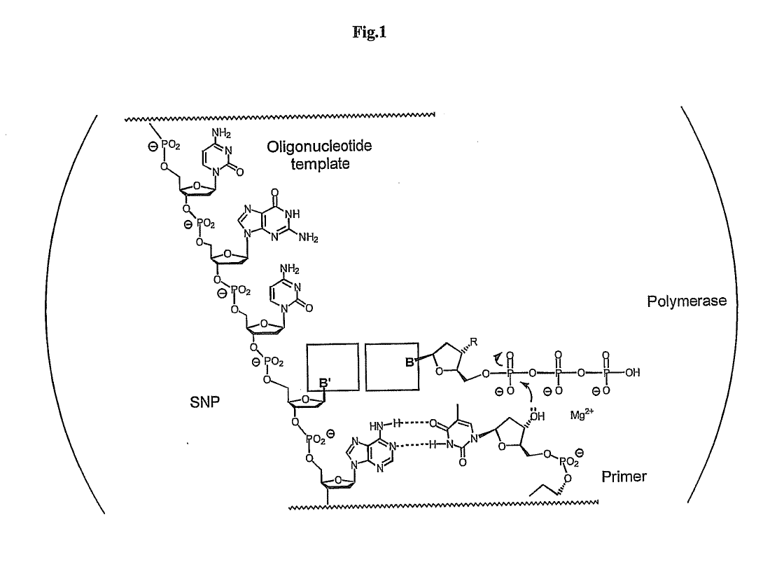

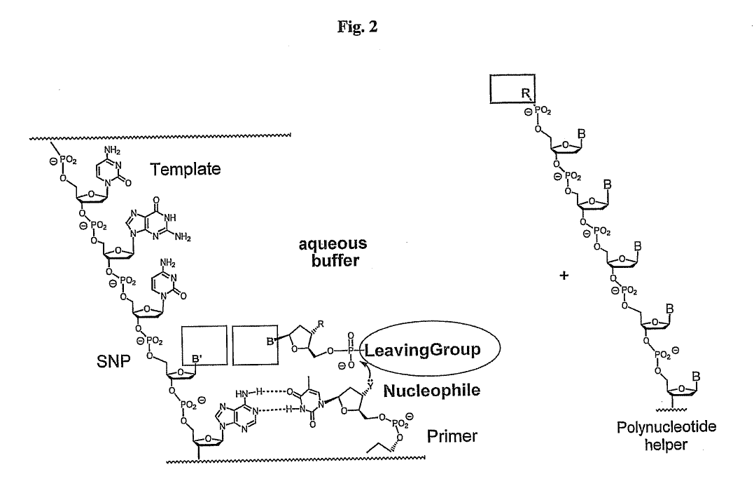

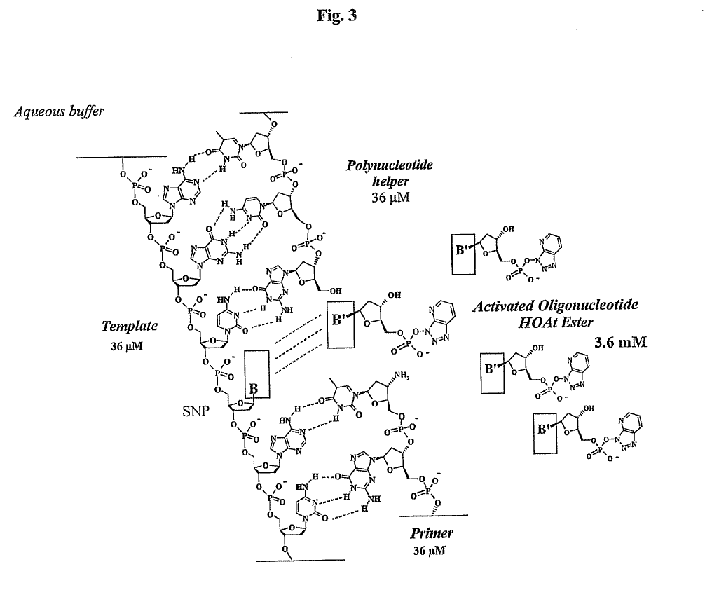

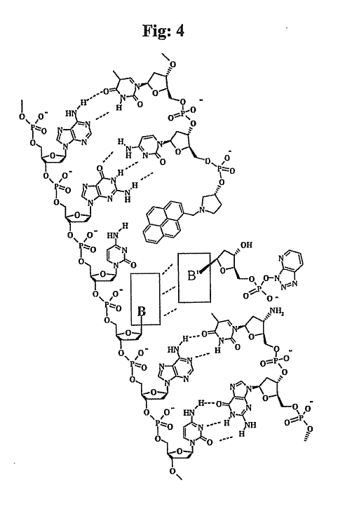

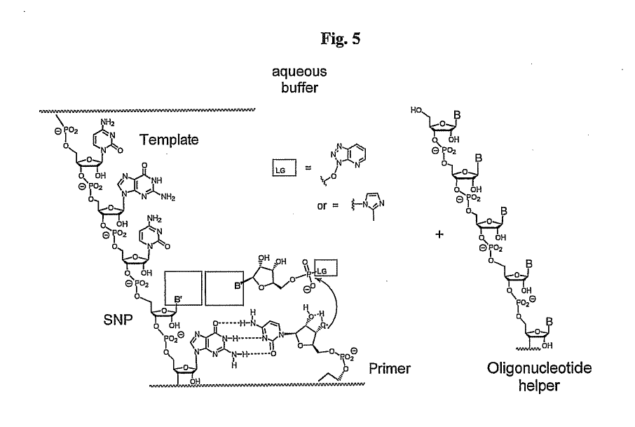

### EXAMPLES

### 1. Synthesis of 1-(2′-Deoxycytidine-5′-O-phosphor-5′-P-yl)-2-azabenzotriazolide

2′-Deoxycytidine-5′-monophosphate (124 μmol, 40 mg) in 5 ml DMF was treated with 1-hydroxy-7-azabenzotriazole (248 μmol, 33.6 mg), O-(7-azabenzotriazol-1-yl)-N,N,N′,N′-tetramethyluronium hexafluorophosphate (248 μmol, 94.4 mg) and diisopropylethylamine (32 μl, 186 μmol). The suspension was stirred 1 h at room temperature under argon. The product was then precipitated by adding to an ice cold solution of NaClO₄ (46 mg, 0.38 mmol) in dry acetone (23.4 ml) and dry diethylether (14.6 ml). After stirring for 20 min at 0° C., the precipitate was isolated by centrifugation. The solid was washed two times with acetone/Et₂O (1:1, v/v, 10 ml) and two times with acetone (10 ml). After drying at 0.1 Torr overnight, the azabenzotriazolide title compound was obtained as pale yellow solid. It was stored under argon at −80° C. until usage. Yield: 29% ³¹P NMR (500 MHZ, DMSO-d₆) δ=−0.86 ppm.

### 2. Synthesis of 1-(2′-Deoxycytidine-5′-O-phosphor-5′-P-yl)-2-triazolide

A slurry of 2′-deoxycytidine-5′-monophosphate (77 μmol, 25 mg) in 500 μl DMF was treated with 1,2,4-1H-triazole (385 μmol, 26.6 mg), triethylamine (65 μl, 462 μmol), triphenylphosphine (65 mg, 246.5 μmol) and 2,2′-dipyridyldisulfide (54 mg, 246.5 μmol). The suspension was stirred for 1 h at room temperature under argon until all monophosphate is dissolved. The product was then precipitated by adding to an ice-cold solution of NaClO₄ (46 mg, 0.38 mmol) in dry acetone (23.4 ml) and dry diethylether (14.6 ml). After stirring for 20 mm at 0° C., the precipitate was isolated by centrifugation. The solid was washed twice with acetone/Et₂O (1:1, v/v, 10 ml) and twice with acetone (10 ml). After drying at 0.1 Torr for 16 h, the nucleotidic triazolide was obtained as colourless solid. It was stored under argon at −80° C. until usage.

Yield: 43%, TLC (cellulose) (ⁱPrOH/NH₃/H₂O 7:1:2) Rf=0.5

³¹PNMR (202.4 MHz, DMSO-d6) δ=−11.4, −12.6 ppm.

### 3. Synthesis of HOAt-activated Guanosinmonophosphate (HOAt-rGMP)

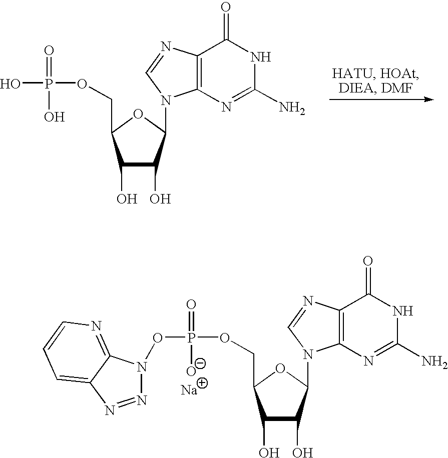

The solids rGMP (1 eq, 0.275 mmol, 100 mg), HATU (1.5 eq, 0.413 mmol, 157.0 mg) and HOAt (1.5 eq, 0.413 mmol, 56.2 mg) were dried 0.1 Torr for approx. 2 h, flooded several times with argon and dissolved in DMF (4.6 ml) to give a clear, colourless solution. DIEA (1.5 eq, 0.413 mmol, 68 μl) was added and the reaction mixture was stirred under Ar atmosphere at room temperature for 5 h. A white solid was removed by centrifugation, and the supernatant was harvested. The product was obtained as a white solid by dropwise addition of the supernatant solution to a cooled solution of NaClO₄ (0.01 M) in acetone/diethylether (1.4:1, v/v, 100 ml). The precipitate was washed twice with acetone/diethylether (1:1, v/v, 50 ml), centrifuged and dried at 0.1 Torr. The product was obtained as a white solid (0.135 mmol, 68.0 mg, 49%).

### 4. Synthesis of Activated Fluorophore-Labeled Mononucleotide

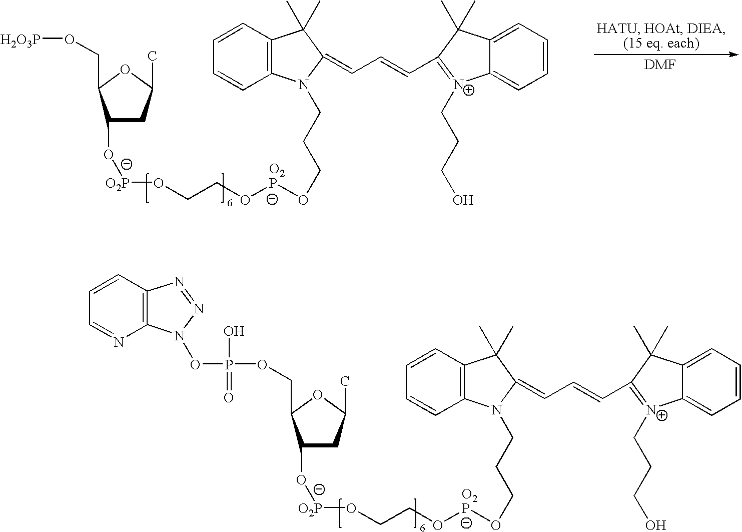

To a stirred solution of the nucleotide (200 mmol) in dry DMF (3.3 μL) was added 6 μL (3 μmol) of a 0.5 μM solution of HATU in dry DMF, 6 μL (3 μmol) of a 0.5 μM solution of HOAt in dry DMF, 6 μl (3 μmol) of a 0.5 μM solution of DIEA in dry DMF. After stirring for 2 h at r.t. to the red solution was added 200 μl of a 10 mM NaOCl₄ solution (27 mg, 0.218 mmol) in dry acetone (18 ml) and dry ether (9 ml). After 1 h at r.t. the precipitate was harvested by removing the supernatant, yielding the HOAt-activated monomer in quantitative yield.

### 5. Synthesis of HATU Activated Nucleotides

| Substance | Mr (g/mol) | Molar equivalents | Amount |
|---|---|---|---|
| Thymidine-5′-monophosphate | 322.20 | 0.062 mmol (1 equ.) | 20.0 mg |
| (TMP) | | | |
| (1-Hydroxy-7-azabenzotriazole) | 136.10 | 0.124 mmol (2 equ.) | 16.6 mg |
| (HOAt) | | | |
| O-(7-Azabenzotrizole-1-yl)- | 380.23 | 0.124 mmol (2 equ.) | 46.0 mg |
| N′,N′,N′,N′-tetramethyl- | | | |
| uronium-hexafluoro-phosphate | | | |
| (HATU) | | | |
| N,N-Diisopropylethylamine | 129.24 | 0.093 mmol (1.5 equ.) | 16.0 μl |
| (DIEA) | | | |

TMP, HOAt and HATU were place in a 5 ml flask and dried for 1 h at 0.1 Torr. The educts were dissolved in 0.6 ml absolute DMF and DIEA was added. Then the reaction mix was stirred under argon for 1 h at room temperature. The reaction product was precipitated by addition to a solution of NaClO₄. The NaClO₄ solution had been prepared by adding 46 mg NaClO₄ to 23.4 ml dry acetone and 14.6 ml dry ether. The precipitate was isolated by centrifugation. The solid was washed three times with acetone/Et₂O (1:1) (3×3 ml) and then with acetone (3×3 ml). Then it was dried at 0.1 Torr.

Yield: 14.3 mg; about 90%.

Activated dAMP, dCMP und dGMP were generated in similar reactions webmail.1und1.de, however, 3 ml DMF were used for dissolving the educts. The educts were not always completely dissolved at the beginning of the reaction.

### 6. Synthesis of 3′-Aminothymidine Primer

Aminothymidine primers with the following sequences were synthesised:

5′-CGCACGT*-3′
and
5′-TCGCAGT*-3′.

Residues represented by letters followed by an asterisk as T* is 3′-amino-thymidine.

The synthesis of phosphoramidate-containing, protected dinucleotide with T* was prepared as described in Rojas et al. (2001) J. Am. Chem. Soc. 123: 12718. The DNA-Synthesis was performed on a Perspective Biosystem 8909 Expedite DNA synthesizer using β-cyanoethyl-phosphoramidites and the standard protocol for 1 μmol scale synthesis recommended by the manufacturer. Reagents were from Proligo (Hamburg, Germany). The solid support bound primer was liberated by treating the solid support with ammonium hydroxide (30% aqueous NH₃, 500 μl) overnight. Crude primer was purified with Poly-PAK™ cartridges from Glen Research (Sterling, USA) using the DMT-on procedure following the manufacturer protocol. The combined eluted fractions were lyophilized and treated with a mixture of acetic acid and water (200 μl, 4:1) at room temperature or at 4° C. The hydrolysis of the phosphoramidate linkage was monitored via MALDI-TOF mass spectrometry and stopped after 3648 h when greater than 90% of the starting material was converted. The reaction was stopped by addition of ammonium hydroxide (30% aqueous NH₃), lyophilised to dryness and purified by HPLC.

For the HPLC purification a 250×10 Nucleosil 120-5 C-18 column (Macherey-Nagel, Düren, Germany) with a gradient of CH₃CN (solvent B) and 0.1 M triethylammonium acetate, pH 7.0 (solution A) at a flow rate of 1 ml/min and detection at 260 nm was used. Primers elute at 17% B. Pure fractions were pooled, lyophilized and stored at −20° C.

Yield: 1-2% by UV quantification after HPLC purification relative to loading of the cpg.

### 7. Primer Extension Reaction with Polynucleotide Helper

The following polynucleotide template was used in the extension reaction:

(SEQ ID NO. 1)

5′-CTG GAT TTC CTC AGC G AC GTG CGT GCC ATT AAA
GTG CGA C-3′

The bold type indicates the position to which a nucleotide has base specifically paired for coupling.

The polynucleotide helper had the following sequence:

| | |
|---|---|
| 3′-GAC CTA AAG GAG TCG-5′ | (SEQ ID NO. 2) |

The polynucleotide primer had the following sequence:

3′-,3*TG CAC GC-5′

Thus, the there annealed polynucleotides had the following structure

5′-CTG GAT TTC CTC AGC G AC GTG CGT GCC ATT AAA
GTG CGA C-3′
3′-GAC CTA AAG GAG TCG *TG CAC GC-5′

Assay conditions:
36 μM polynucleotide template
36 μM polynucleotide primer
36 μM polynucleotide helper (either with or without)
3.6 mM HOAt-dCMP or MeIm-dCMP
in HEPES buffer (200 mM), pH 7.9 with NaCl (200 mM) and MgCl₂ (80 mM)
5 μl total volume
   reaction temperature=20° C.

The solutions of the various components were combined. 0.4 μl aliquots were withdrawn at predetermined time points and transferred into plastic tubes comprising 25 μl H₂O double distilled water and a small amount of cation exchange residue (in its ammonium form). An aliquot of the supernatant of the resulting mixture, e.g. 1 μl, was subsequently analyzed by MALDI-TOF mass spectrometry. If the analysis was not carried out immediately the samples were stored in liquid nitrogen. The results of the extension reactions are depicted in Table 1.

TABLE 1

| d-C derivative | T_{1/2} primer [h] | k^{c} [h − 1] | relative rate |
|---|---|---|---|
| MeIm-dC | 38.5 | 0.018 | 1 |
| MeIm-dC + polynucleotide | 9.5 | 0.073 | 4 |
| helper | | | |
| HOAt-dC | 10 | 0.069 | 1 |
| HOAt-dC + polynucleotide | 1.7 | 0.404 | 5.9 |
| helper | | | |

### 8. Primer Extension Reaction with Polynucleotide Helper and Different Monomers

The polynucleotide template, polynucleotide helper and polynucleotide primer was as indicated above under 7, i.e. (SEQ ID NO. 1).

For sequence specific coupling of nucleotides other than dCMP templates the G residue in SEQ ID NO. 3 in bold type was replaced by A, C, and T, respectively.

Assay conditions:
36 μM polynucleotide template
36 μM polynucleotide primer
36 μM polynucleotide helper
3.6 mM HOAt-dCMP, HOAt-dAMP, HOAt-dGMP or HOAt-dTMP in HEPES buffer (200 mM), pH 7.9 with NaCl (200 mM) and MgCl₂ (80 mM)
5 μl total volume
   reaction temperature 20° C.

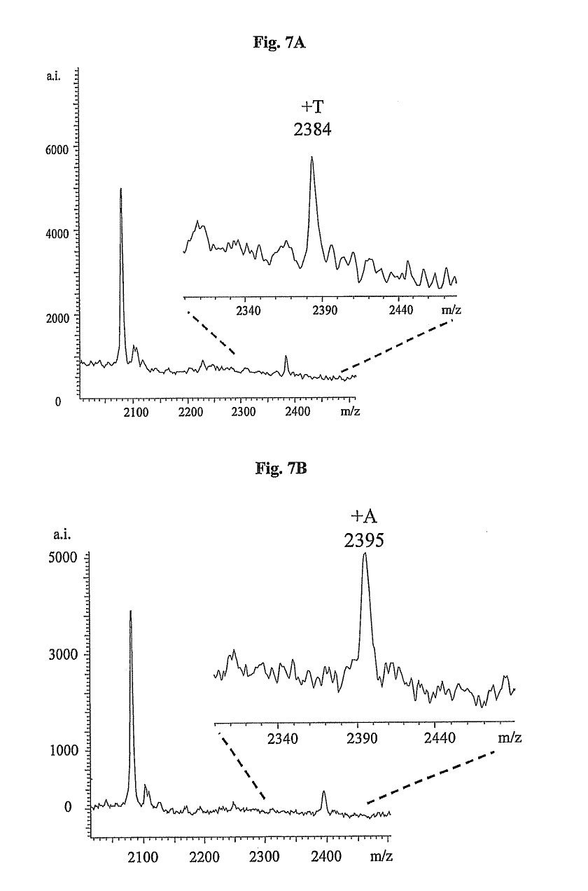

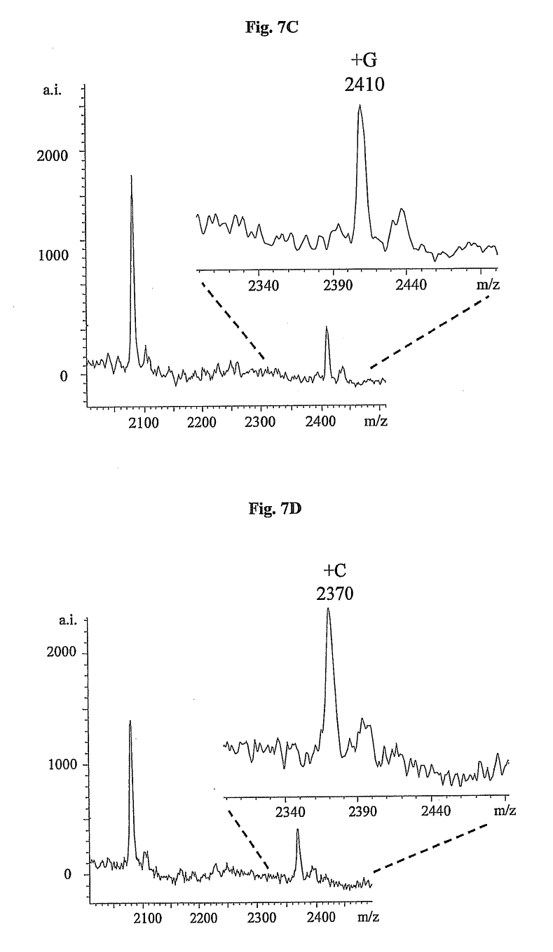

The solutions of the various components were combined. 0.4 μl aliquots were withdrawn at predetermined time points and transferred into plastic tubes comprising 25 μl H₂O double distilled water and a small amount of cation exchange residue (in its ammonium form). An aliquot of the supernatant of the resulting mixture, e.g. 1 μl, was subsequently analyzed by MALDI-TOF mass spectrometry without further purification. If the analysis was not carried out immediately the samples were stored in liquid nitrogen. The result of the extension reaction after the extension had been carried out for 20 min is depicted in FIG. 7 (D). The results of extension reactions involving dAMP-HOAt, dGMP-HOAt and dTMP-HOAt are depicted in FIG. 7 (A) to (C), respectively.

### 9. MALDI-TOF Mass Spectrometry

MALDI-TOF spectra were acquired on a Bruker Reflex IV spectrometer in a linear, negative mode at a total extraction voltage of 20 kV, 18.6 kV delayed extraction (on IS2), and 9.6 V lens voltage from matrix spots prepared from a 2:1 mixture of THAP (0.3 M in EtOH) and diammonium citrate (0.15 M in water) and the analyte solution.

### 10. Comparison of the Rates of Primer Extension Reactions for Different Activation Methods

The following polynucleotide template was used:

| | |
|---|---|
| 5′-TGGTTGACTGCGAT-3′ | (SEQ ID NO. 5) |

The following polynucleotide primer was used (7mer DNA with 3′-terminal amino group):

5′-TCGCAG T*-3′

| | | |
|---|---|---|
| Assay | 36 μM | polynucleotide template |
| conditions: | 36 μM | polynucleotide primer |
| | 3.6 mM | dCMP-derivative in HEPES buffer (200 mM), |
| | | NaCl (400 mM), MgCl₂ (80 mM), |
| | | pH 7.9 total volume 5 μl |

Incubation temperature: 20° C. Aliquots (0.4) μl were withdrawn at stated intervals, diluted 62-fold with deionized water and treated with approx. 100 beads of the ammonium form of Dowex 50W X4, 50-100 mesh cation exchange resin. Samples were immediately frozen in liquid nitrogen and stored at −80° C.

### MALDI-TOF Analysis

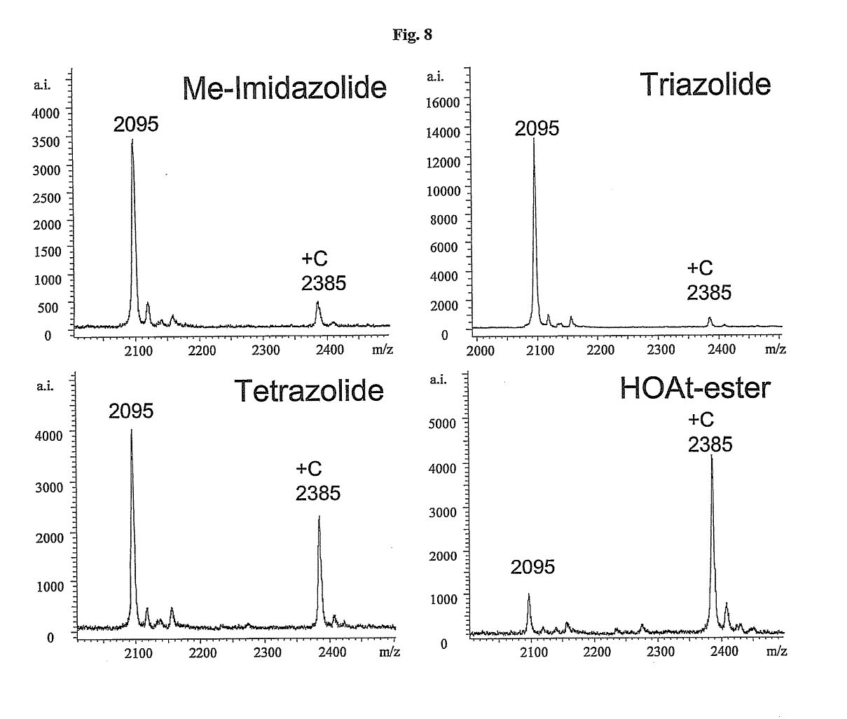

1 μl sample was spotted with 0.5 ml matrix/comatrix mixture (0.3 M THAP in ethanol and 0.15 M diammonium citrate, 2:1) on the anchor chip MALDI target plate (Bruker Daltonics). Two spectra were acquired per data point, using 100 shots from a N₂-laser. The spectra are depicted in FIG. 8. The peak heights were measured and from this data the kinetic data were calculated.

### Kinetic Analysis

The kinetic data were calculated by using a pseudo first order analysis.

For the primer the function f(t)=exp(−a0*t), were a0 is the rate constant to be determined was used. For the extension product the fit function f(t)=1-exp(−a0*t) can be used. Half-life times were calculated from rate constants via t_{1/2}=1n2/kᵢ.

### 1. Experiment

| | t_{1/2}^{primer} | k^{C} | |
|---|---|---|---|
| dCMP-derivative | [h] | [h⁻¹] | relative rate |
| MeIm-dCMP | 17.7 | 0.0393 | 1 |
| HOAt-dCMP | 2 | 0.3537 | 9 |
| Triazole-dCMP | 90.8 | 0.0076 | 0.2 |

### 2. Experiment

| | t_{1/2}^{primer} | k^{C} | |
|---|---|---|---|
| dCMP-derivative | [h] | [h⁻¹] | relative rate |
| MeIm-dCMP | 18.8 | 0.0368 | 1 |
| HOAt-dCMP | 2.1 | 0.3306 | 9 |
| Triazole-dCMP | 83.5 | 0.0083 | 0.2 |
| EDC/dCMP | 36.9 | 0.0188 | 0.5 |

### 11. Primer Extension with EDC and Covalent Catalyst (2-Methylimidazole or 1,2,4-Triazole or Tetrazole)

Polynucleotide template: Metap Template, 60 mer
(part of PCR product frommethionin-amino-
peptidase type2 gene).

(SEQ ID NO. 4)

5′-GAA CGT TCA CTC CAT CGG TCA GTA CCG CAT CGA CGC
TGG TAA AAC CGT TCC GAT CGT-3′
Polynucleotide primer: 3′-T*CATGGC-5
(T* denotes 3′-amino-3′-deoxythymidine residue)

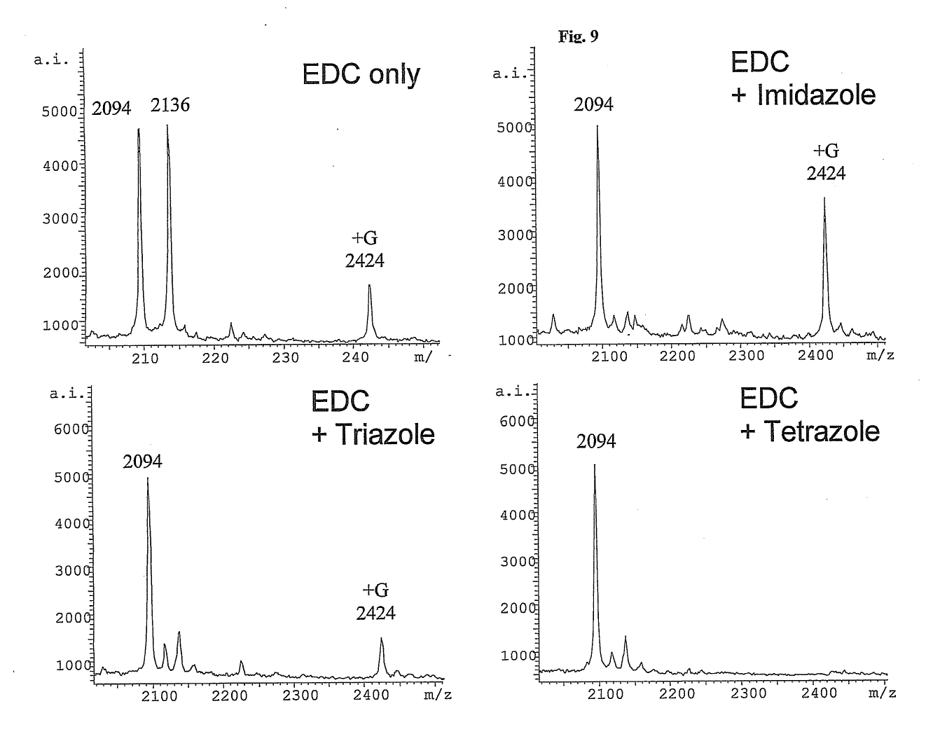

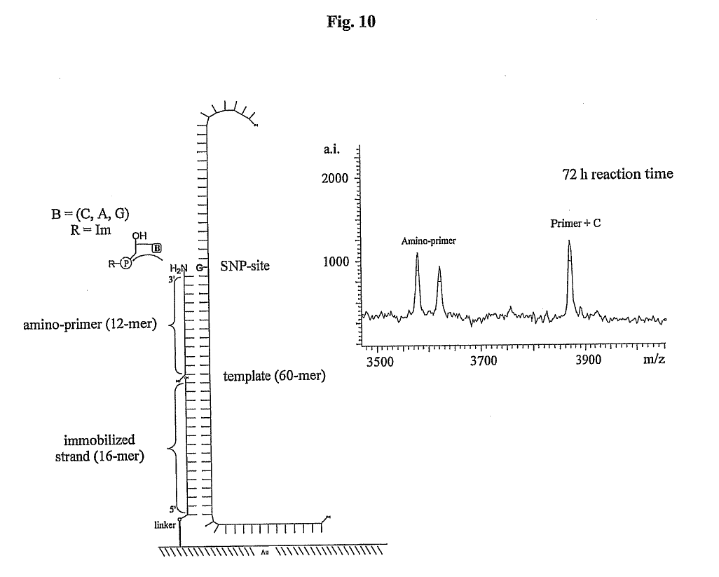

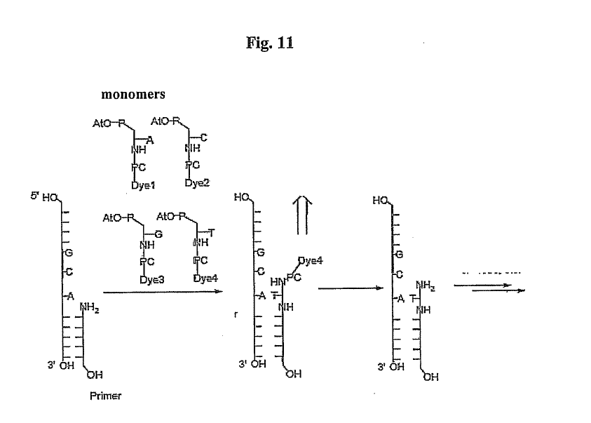

A solution of 0.5 μl of the polynucleotide template (360 μM) and 0.5 μl of polynucleotide primer (360 μM) in 1 μl HEPES Buffer (0.1 M, pH 7.9) was heated to 94° C. in a 200 μl plastic tube (“Eppendorf cup”) for 90 sec and then cooled to 4° C. using a temperature gradient of at 0.1° C./min. Then, 2 μl of a solution of 2′-deoxyguanosine-5′-monophosphate (45 mM), 1 μl of the solution of covalent catalyst (2-methylimidazole or 1,2,4-triazole or tetrazole) (270 mM stock) and 1 μl of an EDC solution (270 mM) were added. The reaction was allows to proceed at 20° C. At various time points, 0.4 μl aliquots of the solution were diluted with 20 μl deionized water containing approx. 1 mg of cation exchange beads Dowex (NH₄⁺ form). The supernatant was used for Maldi-T of analysis. The results of the MALDI-TOF analysis are depicted in FIG. 9.

MALDI-TOF spectra were acquired on a Bruker Reflex IV spectrometer in a linear, negative mode at a total extraction voltage of 20 kV, 18.6 kV delayed extraction (on IS2), and 9.6 V lens voltage from matrix spots prepared from a 2:1 mixture of THAP (0.3 M in EtOH) and diammonium citrate (0.15 M in water) and the analyte solution.

### 12. Template-directed Primer Extension of Oligoribonucleotides with HOAt-activated Monomers and Helper Oligoribonucleotide

(SEQ ID NO. 5)

Polynucleotide template: 5′-CUGGAUUUCCUCAGCAGCACC
G-3′
Polynucleotide primer: 5′-CGGUGC-3′

(SEQ ID NO. 6)

Polynucleotide helper: 5′-GCUGAGGAAAUCCAG-3′.

The nucleotide of the template with which the HOAt-activated monomer base pairs is high ligated by bold type.

The total volume of the assay solution was 5 μl. Assays were performed at 20° C. The oligoribonucleotides (polynucleotide template, primer and helper) were dissolved separately in water to give 1.34 mM stock solutions. For each oligoribonucleotide 1 μl of the stock solution was added to the reaction tube. The stock solution of the aqueous buffer contained HEPES (0.5 M), NaCl (1 M) and MgCl₂ (0.2 M) at pH 7.7. The HOAt-activated monomer (HOAt-rTMP) was dissolved in this buffer to give a 0.05 M solution. From this, 2 μl were added to the solution of the oligoribonucleotides to reach a final concentration of monomer (20 mM), HEPES (200 mM), NaCl (400 mM) and MgCl₂ (80 mM) for the extension reaction. Samples of 0.4 μl volume were taken at a given time and diluted with water to 30.4 μl. The diluted solution were stored over a few grains of an ion exchange resin (NH₄⁺-Dowex) for half an hour before MALDI-TOF MS analysis was performed.

### 13. Non-Enzymatic Elongation Reaction with the HOAt-Activated Fluorophore-Labeled Monomer

(SEQ ID NO. 1)

Polynucleotide template: 5′-CTG GAT TTC CTC AGC
GAC GTG CGT GCC ATT AAA GTG CGA C-3′
Polynucleotide primer: 5′-CGCACGT*-NH₂₋3′
(SEQ ID NO. 2)
Helper oligonucleotide: 5′-GCTGAGGAAATCCAG 3′

Assay conditions:
36 μM polynucleotide template, 36 μM polynucleotide primer, (36 μM polynucleotide helper)
18 mM dCMP-Cy3-HOAt,
in HEPES (200 mM) pH 7.9, NaCl (200 mM), MgCl₂ (80 mM)
5 μl total volume, reaction temperature 20° C.

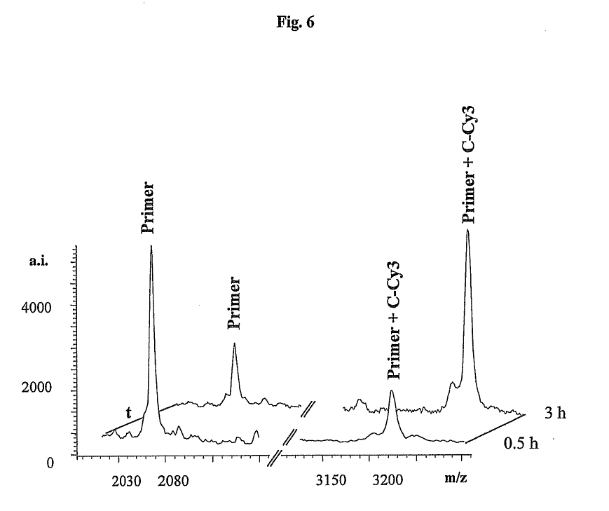

To 0.5 μl (180 μmol) of the template in dH₂O, was added 0.5 μl of a solution of the polynucleotide helper (180 μmol) in dH₂O, 0.5 μl of a solution of the primer (180 μmol) in dH₂O, 2 μL of a 500 mM HEPES buffer solution (NaCl, 500 mM), MgCl₂ (200 mM), 0.65 μl of dH₂O, and 0.85 μl of a solution of the activated mononucleotide (18 mmol) in dH₂O. After stirring the solution at r.t., the progress was analysed via MALDI-TOF mass spectrometry. FIG. 6 depicts the progress of the reaction after 30 min and 3 h.

### 14. Primer Extension on a Microchip, Analyzed in situ by MALDI-TOF Mass Spectrometry

### Preparation of the Molecular System

(SEQ ID NO: 7)

Polynucleotide template: 5′-CAGCGTGAAATTAGGCTGAGAA
CAGAATGATTGATGGTATCTTTTAGGAACCTTTAGGTC-3′

(SEQ ID NO: 8)

Polynucleotide capture probe: 5′-TAAAAGATACCATCAA-
3′

(SEQ ID NO: 9)

Polynucleotide primer: 5′-TCATTCTGTTCT*-3′

A polynucleotide capture probe was immobilized on a quarz slide (12×12 mm) with an intermediate chromium layer (2.5 nm) and a terminal gold layer (250 nm). Thus, in this system the capture probe captures the polynucleotide template. The immobilization conditions are those described in: U. Plutowsld, C. Richert, “A Direct Glimpse of Cross-Hybridization: Background-Passified Microarrays that Allow Mass Spectrometric Detection of Captured Oligonucleotides” Angew. Chem., (published online on Dec. 13, 2004).

In a typical assay employing the microchip thus prepared, a DNA template (2 μM) was hybridized to the immobilized polynucleotide helper capture strand in NH₄OAc-buffer (0.25 M) for 24 hours. Then, after a short washing step with 2 ml of 1 M NH₄OAc-buffer, the 3′-amino-terminal polynucleotide primer (2 μM) in NH₄OAc-buffer (0.25 M) was hybridized to the captured target strand for 10 h to establish a complex consisting of three DNA strands. If a polynucleotide helper is used the nucleotide of the template interrogated by the primer extension reaction is located between the 3′-terminus of the primer and the 5′-terminus of the helper nucleotide in the complex.

### Extension Reaction

Aliquots of 0.5 μl of stock solutions (80 mM) of each of the four imidazolides (dA, dC, dG, dT) were applied to the spots where the DNA had been immobilized on the slide. The stock solutions were in (0.25 M ammonium acetate, pH 7). The mixture was kept at 8° C. while performing the extension reaction. After 72 h, the slide was washed with 2 ml of 1 M NH₄OAc-buffer and subsequently shaken for 2 min on a laboratory shaker (Heidolph, Vibramax) under buffer at 450 rpm. The slide was dried in a stream of argon and attached to the surface of a MALDI-TOF-MS target, followed by spotting 0.1 μl of a mixture of matrix/comatrix solution made up of trihydroxyacetophenon (0.3 M in EtOH) diammonium citrate (0.15 M in H₂O) and subjected to direct mass spectrometric analysis.

### 15. Synthesis of 3′-Fluoresceinylthymidine Building Blocks and Their Implementation

a) 6-Carboxyfluorescein-3′,6′-dipivalat

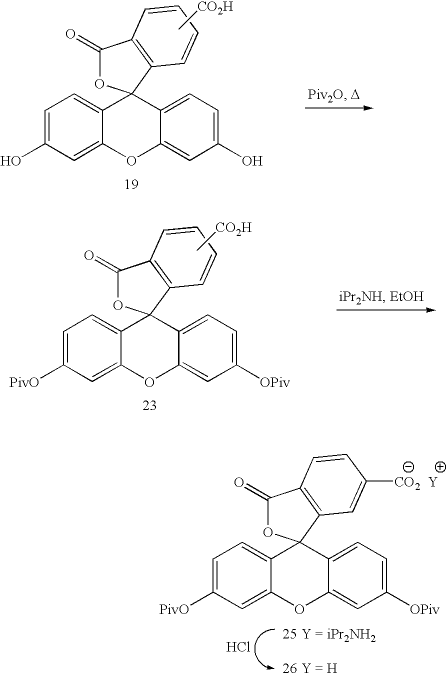

Compound 26 was synthesized according to Rossi and Kao (1997) Bioconjugate Chem. 8: 495-497. The spectroscopic data was consistent with the published data. Yield: 35%.

DC (CH₂Cl₂/MeOH 4:1) R_{f}=0.42.

b) 6-Carboxyfluorescein-3′,6′-dipivalat NHS-ester

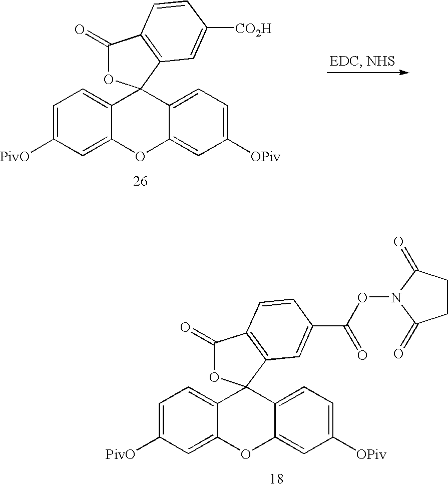

Compound 26 was synthesised analogous to the literature method (Laurent et al. (1997) δ: 856-861) which, however, used a mixture of the isomers. The crude product was used in the next synthesis step without any further purification. Yield: 92%.

DC (CH₂Cl₂/MeOH 98:2) R_{f}=0.35.

### c) 3′-Amino-3′-deoxythymidine

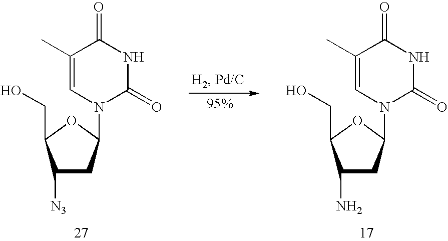

Compound 17 was produced according to published methods (see Lin and Prusoff (1979) J. Med. Chem. 21:109-112). The crude product was used after filtration without any further purification in the next reaction step. Spectroscopic data was consistent with published data. Yield: 95%. DC (MeOH)R_{f}=0.1.

### d) N-(Dipivaloylfluorescein-6-ylcarbonyl)-3′-amino-3′-deoxythymidine

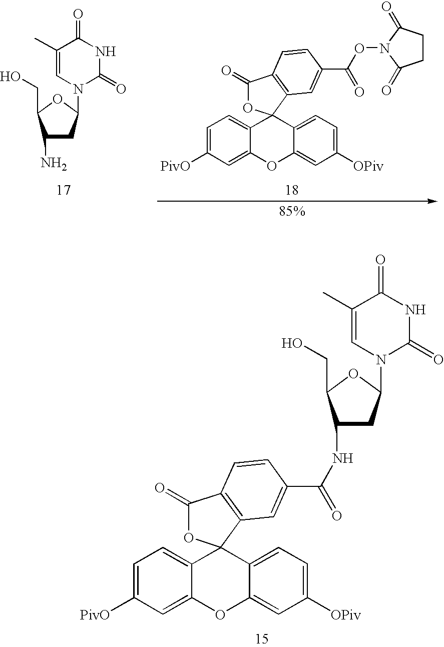

The amine 17 (230 mg, 0.954 mmol=was dissolved in dry DMF (10 mL). While stirring at r.t. portions of the NHS-ester (916 mg, 1.19 mmol) were added. After further stirring at r.t. CH₂Cl₂ (15 mL) was added to the orange solution. The organic phase was washed with NaHCO₃ (2×10 mL). The organic layer was dried on Na₂SO₄ and the solvent removed in vacuo. After chromatography on 100 g silica with a gradient of CH₂Cl₂/MeOH of 95:5 to 9:1 620 mg of compound 15 were obtained (0.807 mmol, yield 85%) as yellowish solid.

DC (CH₂Cl₂/MeOH 9:1) R_{f}=0.35.

### e) N-(Dipivaloylfluorescein-6-ylcarbonyl)-3′-amino-deoxythymidine-5′-O-phosphonate

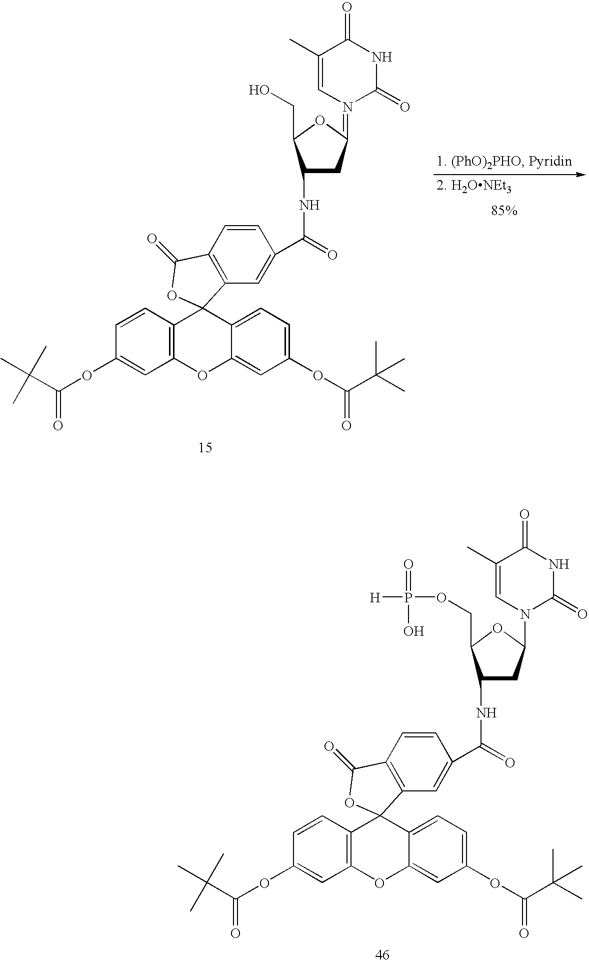

Compound 15 (66 mg, 0.085 mmol) in dry pyridine (1 mL) was slowly added via a syringe to a solution of diphenylphosphit (10 eq., 0.86 mmol, 166 μl). Progress of the reaction was monitored by MALDI-TOF analysis. After 2 h a 1:1 mixture of H₂O/Net₃ (v/) (300 μL) was added bei r.t. After 1 h the solvent was evaporated into a cooling trap at 60° C. and the residue was dried for 1 h in vacuo (HV). The oily residue was dissolved in CH₂Cl₂ and washed twice with TEAB buffer (1 M, triethylammonium hydrogencarbonate, pH=8). The organic phase was dried on Na₂SO₄ and the solvent removed in vacuo (HV). Chromatographic purification on silica with a solvent mixture of CH₂Cl₂/MeOH/AcOH 85:10:5 yielded 61 mg of compound 46 (0.073 mmol; yield: 85%) as slightly yellow oil.

DC (CH₂Cl₂/MeOH/AcOH 85:10:5) R_{f}=0.3.

### f) N-(fluorescein-6-ylcarbonyl)-3′-amino-3′-deoxythymidine-5′-O-phosphorimidazolide sodium

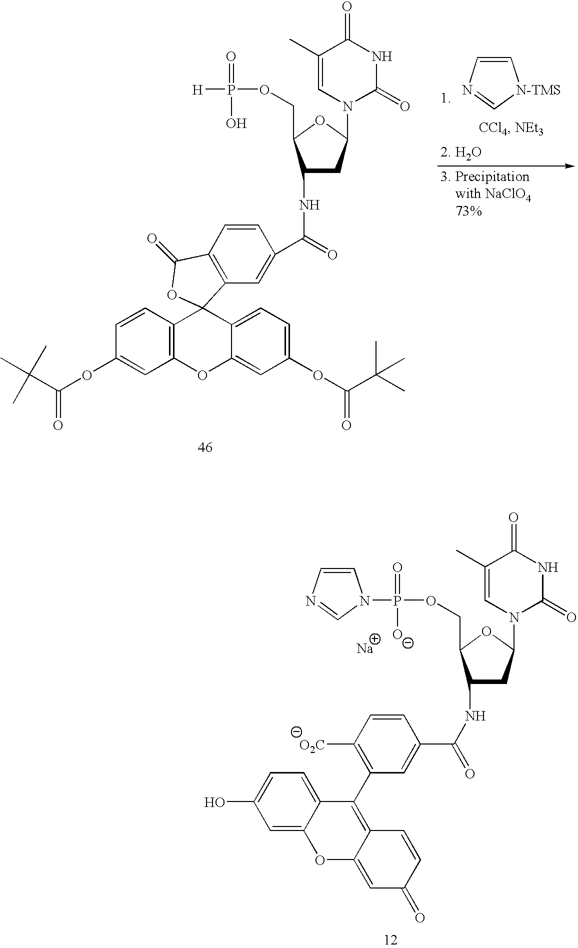

Compound 46 (61 mg, 0.073 mmol) was dissolved in a mixture of DMF/CC₄/Net₃ (1/1/1 v/v, 1.5 ml). TMS-imidazole (5 eq., 55 μL, 0.375 mmol) was dropped into the solution. After 5 h (complete reaction as followed by MALDI-TOF) water was added to the yellowish solution (10 eq., 13.5 μL, 0.75 mmol), which resulted in a dark red colored solution. The complete cleavage of the pivaloyl group was monitored by MALDI-TOF. After 2 h of stirring at r.t. the reaction solution was added in portions into 10 mM: NaClO₄ (27 mg, 0.218 mmol) in absolute acetone (27 mL). After 30 min the dark red precipitate was filtered of and washed with absolute acetone (30 mL). For the isolation of the product the precipitate was washed with H₂O (10 mL) from the glass filter. After freeze drying and further drying under vacuo (HV) 40 mg (0.053 mmol, yield: 73%) of compound 12 resulted as red solid.

DC (CH₂Cl₂/MeOH/Net₃ 47:47:6) R_{f}=0.8

### g) Reacting Compound 17 with Compound 12

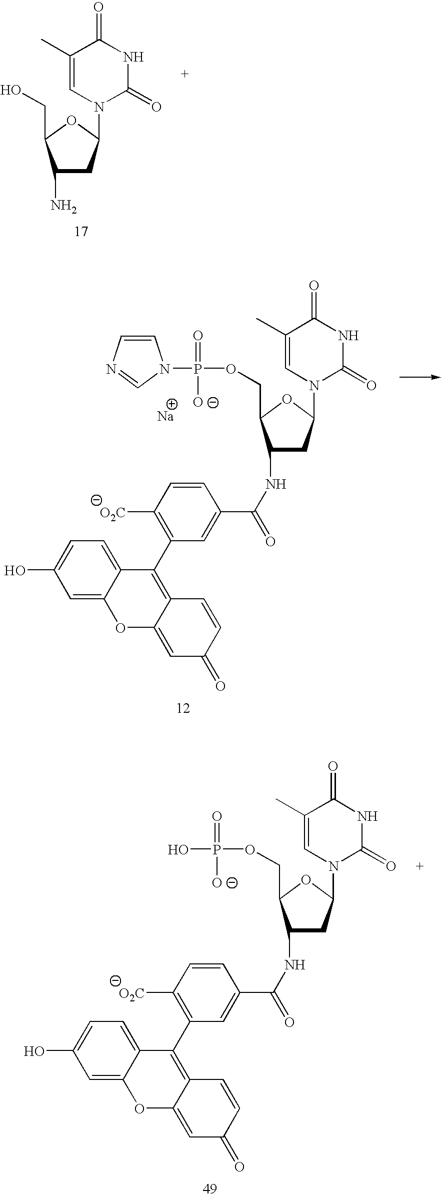

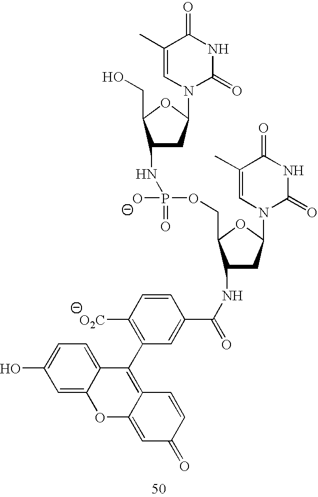

A solution of compound 17 (555 mM, 0.4 μL, 225 nmol) was added to a solution of compound 12 (48.8 in M, 4.6 μL, 224 nmol). The final concentration in the solution was 45 mM of each reactant 12 and 17 in a HEPES buffer (500 mM with NaCl, 1 M and MgCl₂, 200 mM; pH 7.9). The solution was left at r.t. and the reaction progress was monitored by MALDI-TOF.

### 16. Synthesis of a Mononucleotide with Photolabile Linkage of a Cyanine Dye

### a) 5-acetoxy methyl-2-nitroacetophenon

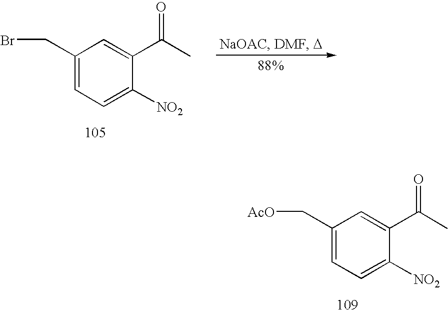

Compound 105 was synthesized starting from 5-methyl-2-nitro-benzoic acid as described by Doppler and Schmid (1979) Helv. Chimica Acta 62: 271-302 to yield 5-methyl-2-nitro acetophenon, which was further reacted to yield 5-bromo-methyl-2-nitro acetophenone as described by Senter et al (1985) Photochem. Photobiol. 42:231-237). Compound 105 (412, 1.59 mmol) was dissolved in DMF (10 mL) and mixed with NaOAc (4 eq. 6.4 mmol, 525 mg) at r.t. The slightly reddish solution was stirred until the reaction was completed, which was indicated by discoloration. The solution was mixed with H₂O (15 mL) and the water phase was extracted with acetic acid ethylester (3 times with 15 mL each). The combined organic phases were washed with NaHCO₃ solution (2 times with 15 mL each) and dried on Na₂SO₄. The organic phase was then dried in vacuo (HV). The crude product (437 mg) was purified by silica chromatography using a solvent gradient hexane/acetic acid ethylester 4:1 (100 mL) to hexane/acetic acid ethylester 3:1 (100 mL) and hexane/acetic acid ethylester 2:1 (400 mL). Compound 109 (332 mg; 1.4 mmol, yield: 88%) was obtained as off-yellow oil, which crystallized to a yellow solid.

DC (hexane/acetic acid ethylester 2:1) R_{f}=0.4.

### b) 5-hydroxymethyl-1-nitroacetophenone

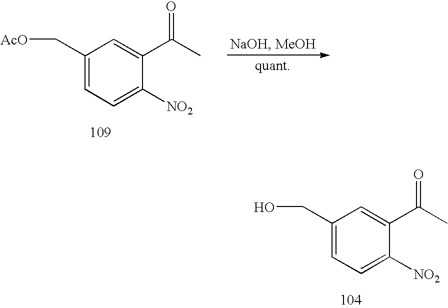

Compound 109 (1.68 g, 7.08 mmol) was dissolved in MeOH (40 mL) and mixed at r.t. with an aqueous NaOH solution (1 M) (2 eq., 14.2 mmol, 14.2 mL). Completion of the reaction was achieved after 2 h. The solution was than neutralized with HCl solution (1M) and extracted with CH₂Cl₂ and dried on Na₂SO₄. After evaporation of the solvent and drying in vacuo the crude compound 104 was obtained, which was further purified by silica chromatography with a solvent gradient CH₂Cl₂/MeOH 99:1 to CH₂Cl₂ 96:4 (200 mL of each). Compound 106 was obtained as a off-yellow solid (1.32 g, 6.76 nmol, yield 96%).

DC (CH₂Cl₂/MeOH 95:5) R_{f}=0.5.

### c) 5-O-(4,4′-dimethoxytriphenylmethyl)methyl-2-nitroacetophenone

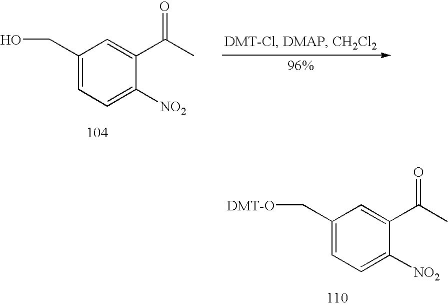

Compound 104 (780 mg, 4 mmol) was dissolved in abs. CH₂Cl₂ (40 mL) and triethylamine (2.5 eq., 10 mmol, 1.41 mL) was added. The solids DMT-Cl (2.5. eq. 10 mmol, 3.38 g) and DMAP (1 eq., 1 mmol, 1.22 g), which were both dried prior to use in vacuo (HV) were added. The reaction was completed after 5 h (DC control). Excess of DMT-Cl was quenched with MeOH (5 eq., 20 mmol, 640 mg, 0.82 mL) for 12 h at r.t. Then H₂O (30 mL) was added to the reaction and the red aqueous phase is extracted with CH₂Cl₂ and the combined organic phase are washed with NaHCO₃ solution and dried on Na₂SO₄. After evaporation of the solvent and drying in vacuo (HV) a reddish oil was obtained. The crude product was recrystallized from EtOH (50 mL) and compound 110 was obtained as yellowish solid (1.91 g, 3.84 mmol, yield: 96%). DC (CH₂Cl₂) R_{f}=0.65.

### d) 4-O-(4,4′dimethoxy-triphenylmethyl)methyl-2-(1-hydroxyethanol)nitrobenzene

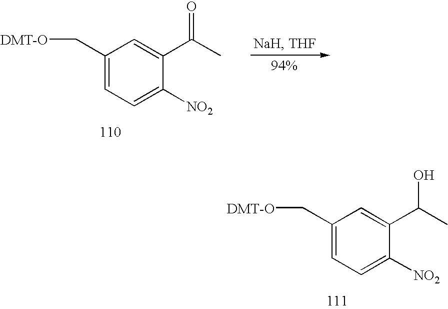

Compound 110 (3.88 g, 7.8 mmol) was dissolved in a mixture of THF (50 mL) and EtOH (50 mL). At r.t. NaBH₄ (4 eq., 31.2 mmol, 1.18 g) was added in one portion. The suspension was stirred under argon for 1 h at r.t. Then acetone (8 eq., 62.4 nmol, 4 g, 4.6 mL) was added to the solution. Upon successive addition of H₂O (40 mL) a white precipitate was formed, which dissolved again upon further addition of H₂O. The solution was extracted several times with CH₂Cl₂ after phase separation with satu. NaCl solution. The combined organic phases were dried on Na₂SO₄, solvent removed and dried in vacuo (HV). The crude product (3.8 g) was purified by silica chromatography using a solvent gradient of hexane/acetic acid ethylester 4:1 (250 mL) to hexane/acetic acid ethylester 3:1 (400 mL) and hexane/acetic acid ethylester 2:1 (900 mL). Compound 111 was obtained as yellow foam (3.65 g, 7.30 mmol, yield: 94%). DC CH₂Cl₂ R_{f}=0.55.

### e) Compound 113

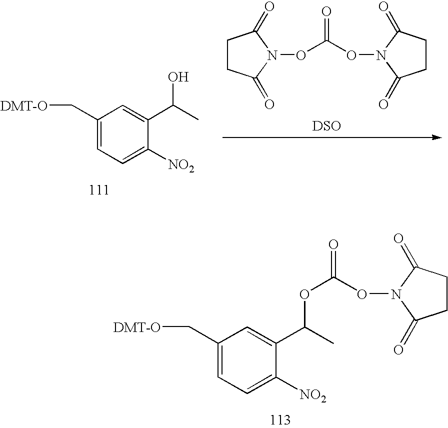

Compound 111 (811 mg, 1.6 mmol) was dissolved in abs. aetonitril (16 mL) and triethylamine (0.69 mL, 4.8 mmol) was added. DSC (1.25 g, 4.8 mmol) was successively added as solid to the solution. The solution was stirred under argon for r.t. under the exclusion of light. After 3 h the solvent was evaporated and the brown residue was dissolved in CH₂Cl₂. The organic phase was washed with satur. NaHCO₃ solution (7 times with 40 mL) and with H₂O (5 times with 40 mL). The organic phase was dried on Na₂SO₄, the solvent removed and the solid residue dried in vacuo (HV). Compound 113 (670 mg, 1.1 mmol, yield: 65%) was obtained as colourless foam.

DC CH₂Cl₂/MeOH (95:5) R_{f}=0.25.

### f) 5′HO-dT-PC

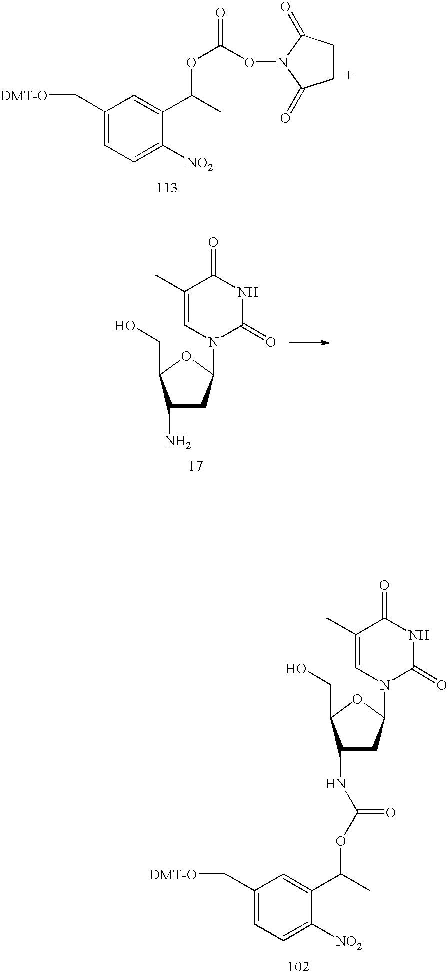

3′-aminothymidine (220 mg, 0.9 mmol) was dissolved in abs. DMF (5 mL) and triethylamine (0.2 mL, 1.41 mmol) was added. Compound 113 (600 mg, 0.94 mmol) was added to the clear solution as solid. The solution was stirred at r.t. for 12 h under the exclusion of light, CH₂Cl₂ (20 mL) was added to the organic phase, which was washed with H₂O (15 mL) and NaHCO₃ solution (2×15 mL). The organic phase was dried on Na₂SO₄, the solvent removed and the solid residue dried in vacuo (HV). The crude product was purified by silica chromatography using CH₂Cl₂/MeOH 97:3 as solvent to obtain compound 102 (655 mg, 0.85 mmol, yield: 91% as yellow foam. DC (CH₂Cl₂/MeOH 97:3) R_{f}=0.2.

### g) 5′-PA-dT-PC

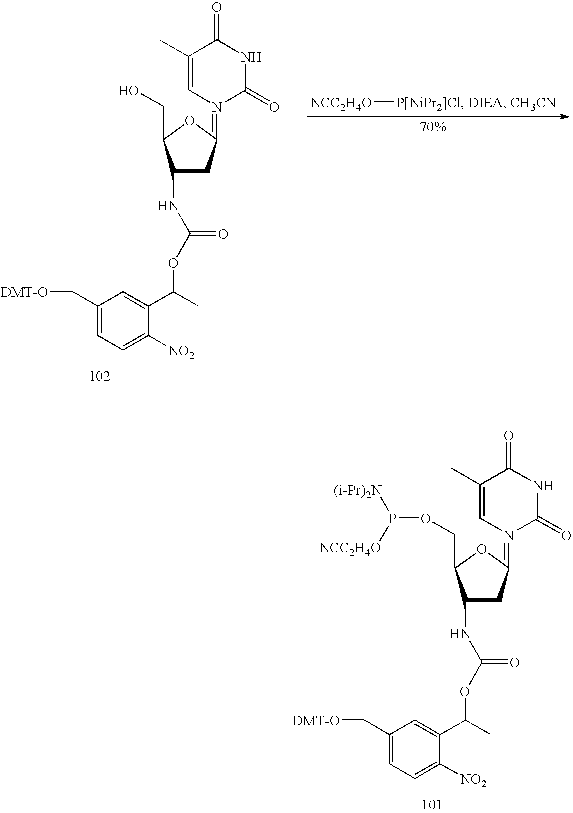

Compound 102 (77 mg, 0.1 μmol) was dried in vacuo (UV) for 12 h under the exclusion of light. Compound 102 was then dissolved in acetonitrile (2 mL) and DIEA (46 μL, 0.28 mmol) was added. The phosphitylation reagent (32 μL, 0.14 mmol) was added at 0° C. and then the cooling was removed. After 1 h additional phosphitylation reagent (32 μL, 0.14 mmol) was added. After further 30 min the solvent was removed and the residue dissolved in CH₂Cl₂ (20 mL). The organic phase was washed with satur. NaHCO₃ (2×20 mL) and dried on Na₂SO₄, the solvent removed and with acetonitrile (2×5 mL) coevaporated. The resulting residue was dried in vacuo (HV) and then dissolved in CH₂Cl₂ (1 mL). The solution was added dropwise to a solution of pentane (30 mL and triethylamine (0.25 mL), which led to the formation of a precipitate. The upper phase was carefully removed and the residue dried in vacuo (HV). Compound 101 was (83 mg, 0.085 mmol, 85%) was obtained as yellowish foam.

DC (CH₂Cl₂/MeOH 95:5) R_{f}=0.35.

### h) P-dT-PC—Cy5

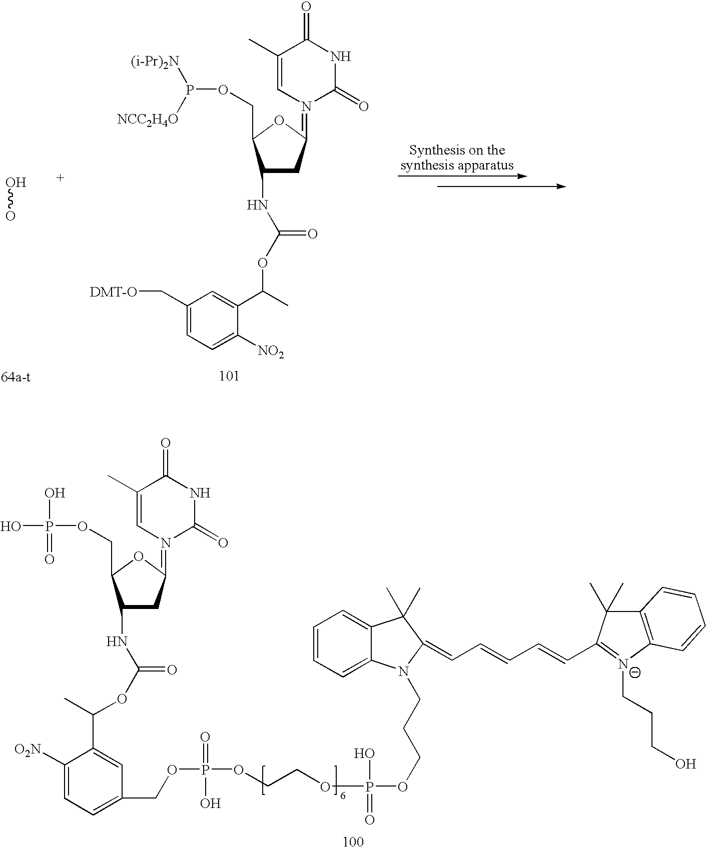

The synthesis was carried out according to standard protocols. The product was eluted off the SepPak cartridge with gradient steps with a percentage of 30-45% acetonitrile. Yield: 5%.

### i) Oat-P-dT-PC-Cy5

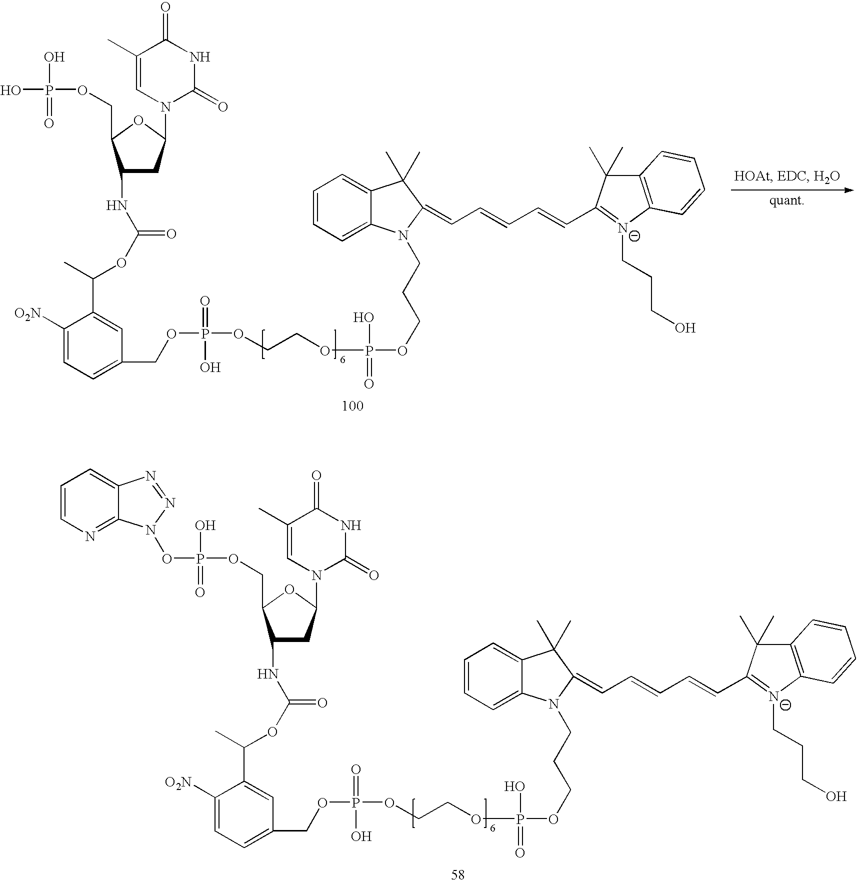

Activation of compound 100 to yield the OAt ester was done as outlined above.

1. Nucleotide having a structure according to formula (I) 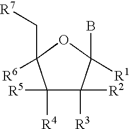
(I),
wherein
R¹ has the meaning H, saturated or unsaturated, linear or branched, C₁ to C₁₀ alkyl, which can be substituted with one or more halogen, OH, NH—CO—R or is a direct or indirect link to a marker residue or a stacking residue;
R² has the meaning H, OH, SH, F, Cl, Br, I, saturated or unsaturated, linear or branched, substituted or unsubstituted C₁ to C₁₀ alkyl, or is a direct or indirect link to a marker residue or a stacking residue;
R³ has the meaning H, OH, SH, F, Cl, Br, I, saturated or unsaturated, linear or branched, unsubstituted or substituted C₁ to C₁₀ alkyl, —NR′R″, is a phosphate group, an activated phosphor ester, an activated carboxylic ester, CHO, COOH, a polynucleotide, a polynucleotide comprising a stacking residue, is a direct or indirect link to a marker residue or a stacking residue or is connected to R⁶ via a C₁ to C₄ alkyl or alkyl ether chain;
R⁴ has the meaning H, OH, SH, F, Cl, Br, I, saturated or unsaturated, linear or branched, unsubstituted or substituted C₁ to C₁₀ alkyl, —NR′R″, is a phosphate group, an activated phosphor ester, an activated carboxylic ester, CHO, COOH, a polynucleotide, a polynucleotide comprising a stacking residue or is a direct or indirect link to a marker residue or a stacking residue;
R⁵ has the meaning H, OH, SH, F, Cl, Br, I, saturated or unsaturated, linear or branched, substituted or unsubstituted C₁ to C₁₀ alkyl, or is a direct or indirect link to a marker residue or a stacking residue;
R⁶ has the meaning H, saturated or unsaturated, linear or branched, C₁ to C₁₀ alkyl, which can be substituted with one or more halogen, OH, NH—CO—R, is a direct or indirect link to a marker residue or a stacking residue or is connected to R³ via a C₁ to C₄ alkyl or alkyl ether chain;
R⁷ has the meaning H, OH, SH, F, Cl, Br, I, saturated or unsaturated, linear or branched, unsubstituted or substituted C₁ to C₁₀ alkyl, —NR′R″, is a phosphate group, an activated phosphor ester, an activated carboxylic ester, CHO, COOH, a polynucleotide, a polynucleotide comprising a stacking residue or is a direct or indirect link to a marker residue or a stacking residue,
wherein R has the meaning H, saturated or unsaturated, linear or branched, unsubstituted or substituted alkyl, saturated or unsaturated, unsubstituted or substituted cycloalkyl, or unsubstituted or substituted aryl or heteroaryl, and
R′ and R″ independent of each other have the meaning H, saturated or unsaturated, linear or branched, unsubstituted or substituted alkyl, saturated or unsaturated, unsubstituted or substituted cycloalkyl, unsubstituted or substituted aryl or heteroaryl,
B is a purine or pyrimidine base or base analog or a purine, a pyrimidine or base analog comprising a stacking and/or a marker residue
under the proviso that one of R³, R⁴, and R⁷ is an activated phosphor ester or an activated carboxylic ester and under the proviso that when R¹, R², R⁵, R⁶ is H, R³, R⁴ is OH and B is A, G, C, T or U than R⁷ is not phosphoro-2-methylimidazolid.

2. Nucleotide according to claim 1, wherein R¹, R², R⁵ and R⁶ have the meaning H.

3. Nucleotide according to claim 1, wherein R³, R⁴ and R⁷ independent of each other have the meaning H, OH, NR′R″ or are a direct or indirect link to a marker residue under the proviso that one of R³, R⁴, and R⁷ is an activated phosphor ester or activated carboxylic ester.

4. Nucleotide according to any of claim 1, wherein the activated phosphor ester is selected from the group consisting of structures according to formulas (II) to (XIX) 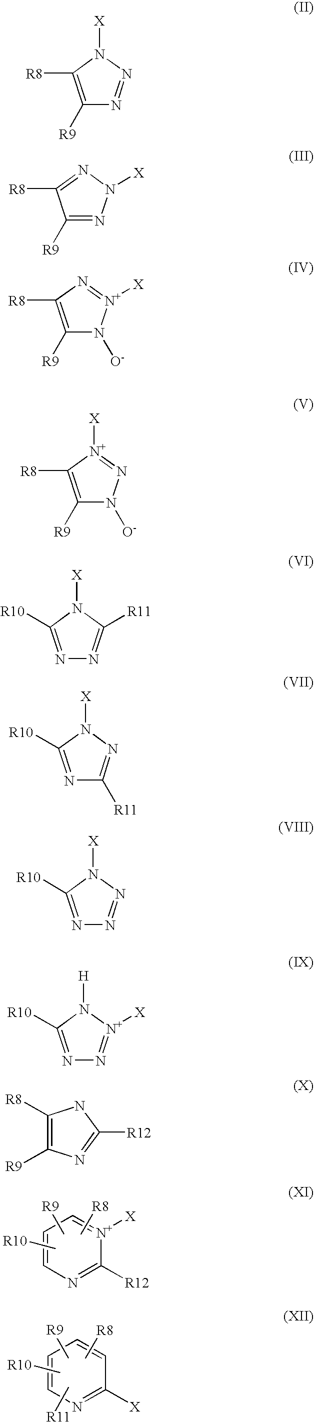 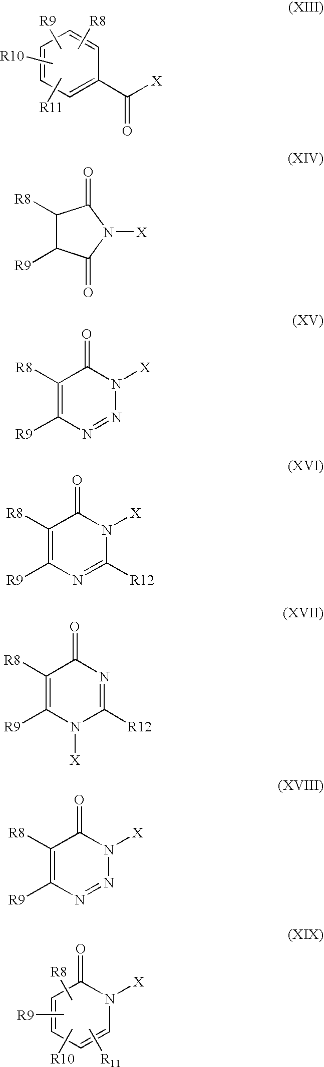
wherein R⁸ and R⁹ independent of each other have the meaning H, OH, SH, F, Cl, Br, I, CN, NO₂, saturated or unsaturated, linear or branched, unsubstituted or substituted C₁ to C₅ alkyl, or taken together form a saturated or unsaturated, unsubstituted or substituted mono, bi or polycyclic ring;
R¹⁰ and R¹¹ independent of each other have the meaning H, OH, SH, F, Cl, Br, I, CN, NO₂, saturated or unsaturated, linear or branched, unsubstituted or substituted C₁ to C₅ alkyl;
R¹² has the meaning H, OH, SH, F, Cl, Br, I, CN, NO₂, CH₃, substituted methyl, saturated or unsaturated, linear or branched, unsubstituted or substituted C₂ to C₅ alkyl,
and X is selected from the group consisting of the structures according to formulas (XX) to (XXVII) 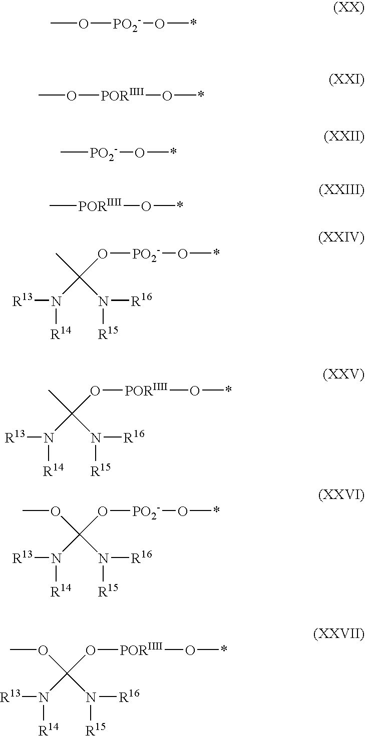
wherein * designates the bond of the activated phosphate ester to the sugar moiety within the nucleotide,
R¹³ and R¹⁶ independent of each other have the meaning H, linear or branched, substituted or unsubstituted C₁ to C₁₀ alkyl, linear or branched C₁ to C₁₀ alkyl-NR¹⁷R¹⁸, wherein R¹⁷ and R¹⁸ independent of each other mean linear or branched substituted or unsubstituted C₁ to C₅ alkyl, C₃ to C₈ cycloalkyl, aryl, or heteroaryl;
R¹⁴ and R¹⁵ either mean a free electron pair or R¹³ and R¹⁴ and/or R¹⁵ and R¹⁶ together form a heteroaryl; and
R^{IIII} has the meaning saturated or unsaturated, linear or branched alkyl, aryl or heteroaryl, which can be substituted one or more times with OH, SH, NH₂, F, Cl, Br or I.

5. Nucleotide according to claim 4, wherein the activated phosphor ester is selected from a group consisting of structures according to formulas (XXVIII) to (XXXIX) 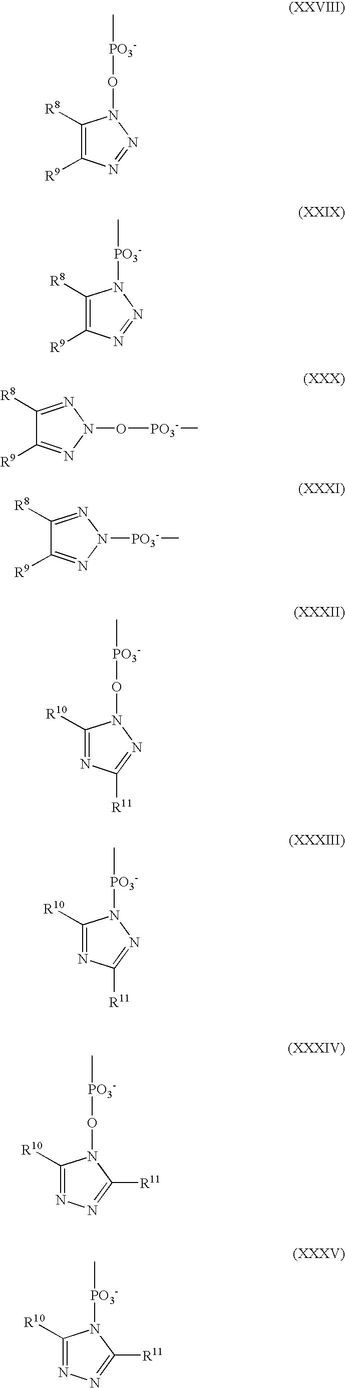 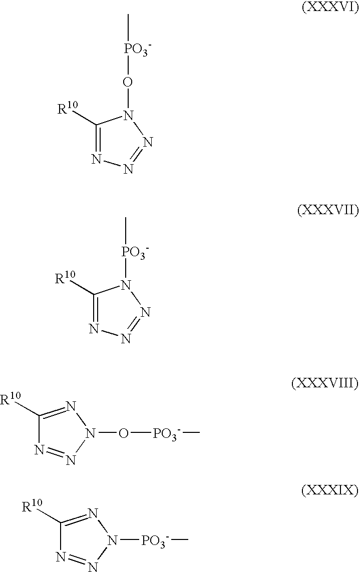
wherein R⁸ and R⁹ independent of each other have the meaning H, OH, SH, NH₂, F, Cl, CN, NO₂; Br, I, saturated or unsaturated, linear or branched, unsubstituted or substituted C₁ to C₅ alkyl, or taken together form a saturated or unsaturated, unsubstituted or substituted mono, bi or polycyclic ring;
and R¹⁰ and R¹¹ independent of each other have the meaning H, OH, SH, NH₂, F, Cl, Br, I, CN, NO₂, saturated or unsaturated, linear or branched, unsubstituted or substituted C₁ to C₅ alkyl.

6. Nucleotide according to claim 1, wherein R⁸ and R⁹ together form an unsubstituted or substituted aromatic or heteroaromatic mono or bicyclic ring.

7. Nucleotide according to claim 1, wherein the activated phosphate ester with a structure according to:
a) formula (XXVIII) is selected from the group consisting of 6-chloro-1-hydroxybenzotriazole phosphate, 1-hydroxybenzotriazole phosphate, 1-hydroxyazabenzotriazole phosphate and 1-hydroxytriazol phosphate;
b) formula (XXIX) is selected from the group consisting of benzotriazole phosphate, 6-chlorobenzotriazole phosphate, azabenzotriazole phosphate, and triazole phosphate;
c) formula (XXX) is selected from the group consisting of 6-chloro-2-hydroxybenzotriazole phosphate, 2-hydroxybenzotriazole phosphate, 2-hydroxyazabenzotriazole phosphate and 2-hydroxytriazol phosphate;
d) formula (XXXI) is selected from the group consisting of benzotriazole phosphate, 6-chlorobenzotriazole phosphate, azabenzotriazole phosphate, and triazole phosphate;
e) formula (XXXII) is selected from the group consisting of 1-hydroxytriazole phosphate, and 5-chloro-1-hydroxytriazole phosphate;
f) formula (XXXIII) is selected from the group consisting of triazole phosphate, 5-chloro-triazole phosphate;
g) formula (XXXIV) is selected from the group consisting of 1-hydroxytriazole phosphate, and 2-chloro-1-hydroxytriazole phosphate;
h) formula (XXXV) is selected from the group consisting of triazole phosphate, and 2-chloro-triazole phosphate;
i) formula (XXXVI) is selected from the group consisting of 1-hydroxytetrazole phosphate and 5-chloro-1-hydroxytetrazole phosphate;
j formula (XXXVII) is selected from the group consisting of tetrazole phosphate and 5-chloro-tetrazole phosphate;
k) formula (XXXVIII) is selected from the group consisting of 2-hydroxytetrazole phosphate and 5-chloro-2-hydroxytetrazole phosphate; and
l) formula (XXXIX) is selected from the group consisting of tetrazole phosphate and 5-chloro-tetrazole phosphate.

8. Nucleotide according to claim 1, wherein the activated phosphor ester is a pentafluorophenole phosphor ester.

9. Nucleotide according to claim 1, wherein the purine base is selected from the group consisting of adenine, deazaadenine, guanine, deazaguanosine, and inosine or from the respective purine base comprising a marker or stacking residue.

10. Nucleotide according to claim 1, wherein the pyrimidine base is selected from the group consisting of cytosine, thymine, uracil, isocytosine, dihydrouracil, thiouracil, pseudouracil, and 5-methylcytosine or from the respective pyrimidine base comprising a marker or stacking residue.

11. Nucleotide according to claim 9, wherein the stacking residue or marker residue is attached to the 5-position of the pyrimidine base or the 7 or 8 position of the purine base.

12. Nucleotide according to claim 1, wherein the base analog or analog comprising a stacking residue or marker residue is selected from the group consisting of difluorotoluene, and imidazole-4-carboxamide.

13. Nucleotide according to claim 1, wherein the stacking residue is selected from the group consisting of aromatic or heteroaromatic bi, tri or polycyclic ring systems.

14. Nucleotide according to claim 1, wherein the stacking residue is selected from the group consisting of indole, napthol, bile acid, quinoline, quinolone, stilbene, pyrene, a steroid ring system, anthraquinone, an ethidium residue, an anthracene residue, and tetracene, which can be substituted with one or more residues selected from the group consisting of OH, SH, NH₂, F, Cl, Br and I.

15. Nucleotide according to claim 1, wherein the marker is selected from the group consisting of a fluorescent residue, a radioactive residue, a phosphorescent residue, a chelating residue comprising a metal ion and a quenching residue.

16. Nucleotide according to claim 1, wherein the indirect link between the ribose radical depicted in structure (I) and the marker and/or the stacking residue is a photo cleavable linker.

17. Method of polymerase independent elongation of a polynucleotide primer comprising the steps of:
a) providing an polynucleotide primer, with at least one 2′, 3′ or 5′ terminal amino group and
b) reacting the polynucleotide primer with a nucleotide according to claim 1.

18. Method of polymerase independent elongation of a polynucleotide primer comprising the steps of:
a) providing an polynucleotide primer, with at least one 2′, 3′ or 5′ terminal amino group and
b) reacting the polynucleotide primer with a nucleotide or a polynucleotide with at least one 2′, 3′ or 5′ terminal phosphate or carboxy residue, which has been activated with an activating reagent.

19. Method according to claim 18, wherein the activating reagent is selected from a pentafluorophenyl ester reagent, a phosphonium reagent, an uronium reagent, or an acid fluoride reagent.

20. Method according to claim 18, wherein the activating reagent is selected from the group comprising 2-chloro-1,1,3,3-tetramethyluronoium hexachloroantimonate (ACTU), O-(7-azabenzotriazol-1-yl)-1,1,3,3-tetramethyluronoium hexafluorophosphate (HATU), 2-(1H-benzotriazol-1-yl)-1,1,3,3-tetramethyluronoium hexafluorophosphate (HBTU), O-(1H-6-chlorobenzotriazole-1-yl)-1,1,3,3-tetramethyluronium hexafluorophosphate (HCTU), O-(7-azabenzotriazol-1-yl)-bis(pyrrolidin-1-yl)methylium hexafluorophosphate (HAPyU), 2-(1H-benzotriazol-1-yl)-bis(pyrrolidin-1-yl)methylium hexafluorophosphate (HBPyU), O-(1H-6-chlorobenzotriazole-1-yl)-bis(pyrrolidin-1-yl)methylium hexafluorophosphate (HCPyU), 2-(1H-benzotriazole-1-yl)-1,1,3,3-tetramethyluronium tetrafluoroborate (TBTU), O-(1H-6-chlorobenzotriazole-1-yl)-1,1,3,3-tetramethyluronium tetrafluoroborate (TCTU), 2-(endo-5-norbornene-2,3-dicarboxymido)-1,1,3,3-tetramethyluronium tetrafluoroborate (TNTU), O-(1,2-dihydro-2-oxo-pyridyl]-N,N,N′,N′-tetramethyluronium tetrafluoroborate (TPTU), 2-succinimido-1,1,3,3-tetramethyluronium hexafluorophosphate (HSTU), 2-succinimido-1,1,3,3-tetramethyluronium tetrafluoroborate (TSTU), pentafluorphenol-tetramethyluronium hexafluorophosphat (PFTU), N,N,N′,N′-tetramethyl-fluoroformamidinium hexafluorophosphate (TFFH), N,N,N′,N′-tetramethyl-chloroformamidinium-hexafluorophosphate (TCFH), O-(cyano-(ethoxycarbonyl)-methylenamino)-1,1,3,3-tetramethyluronium tetrafluoroborate (TOTU), N-hydroxy-5-norbene-endo-2,3-dicarboxamide (HONB), pentafluoro-phenyl-trifluoroacetat, pentafluorophenyl diphenylphosphinate (FDPP), (PfPyU), (PfTU), O-(7-azabenzotriazol-1-yl)-tris(dimethylamino)-phosphonium hexafluorophosphate (AOP), 2-(1H-benzotriazol-1-yl)-tris(dimethylamino)-phosphonium hexafluorophosphate (BOP), O-(1H-6-chlorobenzotriazole-1-yl)-tris-(dimethylamino)-phosphonium hexafluorophos-phate (COP), 7-azobenzotriazolyoxy-tris(pyrrolidino) phosphonium hexafluorophosphate (PyAOP), 1-benzotriazolyoxy-tris(pyrrolidino) phosphonium hexafluorophosphate (PyBOP), tris(pyrrolidino) phosphonium hexafluorophosphate (PyCOP), tetramethyl-fluoroformamidinium hexafluorophosphate (TFFH) and bis(tetramethylene)-fluoroformamidinium hexafluorophosphate (BTFFH).

21. Method according to claim 18, wherein the activating reagent has a structure according to formula (XXXIV) 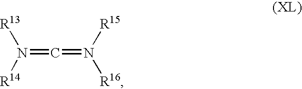
wherein
R¹³ and R¹⁶ independent of each other mean H, linear or branched, substituted or unsubstituted C₁ to C₁₀ alkyl, linear or branched C₁ to C₁₀ alkyl-NR¹⁷R¹⁸, wherein R¹⁷ and R¹⁸ independent of each other mean H, linear or branched substituted or unsubstituted C₁ to C₅ alkyl, C₃ to C₈ cycloalkyl, aryl, or heteroaryl;
R¹⁴ and R¹⁵ either mean a free electron pair or R¹³ and R¹⁴ and/or R¹⁵ and R¹⁶ together form a heteroaryl;
or is 2-fluoro pyridine; R^{V}—CO—Cl; or Z-SO₂—R^{V}, wherein R^{V} has the meaning saturated or unsaturated, C₁ to C₁₀ alkyl, aryl, heteroaryl, which can be substituted with one or more OH, SH, NH₂, F, Cl, Br, or I
and the activation of the nucleotide or polynucleotide is carried out in the presence of a catalyst selected from the group consisting of a structure according to formula (XLI) to (L) 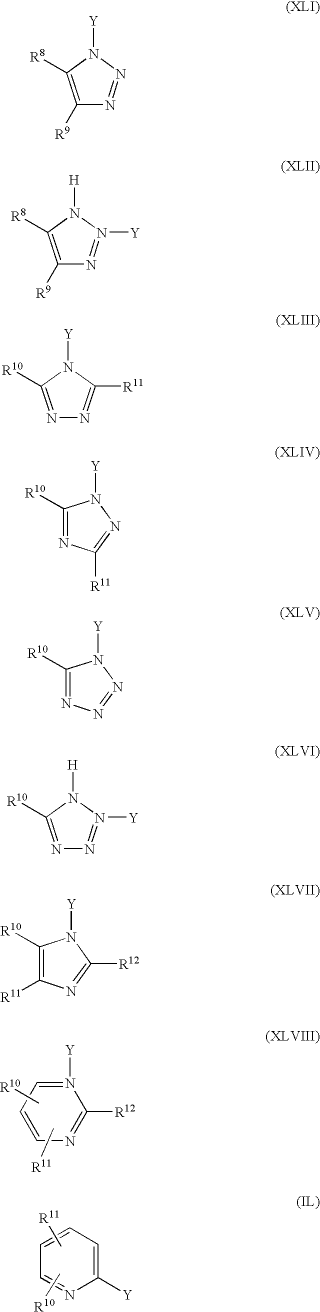 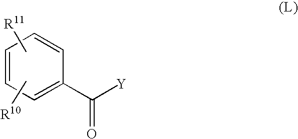
wherein R⁸ and R⁹ independent of each other have the meaning H, OH, SH, NH₂, F, Cl, Br, I, saturated or unsaturated, linear or branched, unsubstituted or substituted C₁ to C₁₀ alkyl, or taken together form a saturated or unsaturated, unsubstituted or substituted mono, bi or polycyclic ring;
R¹⁰ and R¹¹ independent of each other have the meaning H, OH, SH, NH₂, F, Cl, Br, I, saturated or unsaturated, linear or branched, unsubstituted or substituted C₁ to C₁₀ alkyl, linear or branched C₁ to C₁₀ alkyl-NR¹⁹R²⁰, wherein R¹⁹ and R²⁰ independent of each other mean linear or branched substituted or unsubstituted C₁ to C₁₀ alkyl, C₃ to C₈ cycloalkyl, aryl, or heteroaryl;
R¹² has the meaning H, OH, SH, NH₂, F, Cl, Br, I, CH₃, substituted methyl, saturated or unsaturated, linear or branched, unsubstituted or substituted C₂ to C₅ alkyl,
and Y is selected from the group consisting of H and OH.

22. Method according to claim 21, wherein the catalyst is selected from the group consisting of imidazole, methylimidazole, benzimidazole, triazole, tetrazole, hydroxybenzotriazole, azahydroxybenzotriazole, chlorobenzotriazole, dimethylaminopyridine (DMP).

23. Method according to claim 18, wherein the nucleotide or the polynucleotide further comprises a stacking residue and/or a direct or indirect link to a marker residue.

24. Method according to claim 23, wherein the stacking residue is selected from the group consisting of indole, napthol, a steroid ring system, bile acid, quinoline, quinolone, stilbene, pyrene, anthraquinone, an ethidium residue, an anthracene residue, and tetracene, which can be substituted with one or more residues selected from the group consisting of OH, SH, NH₂, F, Cl, Br and I.

25. Method according to claim 23, wherein the marker is selected from a fluorescent residue, a radioactive residue, a phosphorescent residue, a chelating residue comprising a metal ion and a quenching residue.

26. Method according to claim 18 to 25, wherein R¹³ and R¹⁶ independent of each other mean CH₃, C₂H₅, C₃H₇, C(CH₃)₃, C₂H₄N(CH₃)₂, cycloC₆H₁₁ and C₃H₆N(CH₃)₂.

27. Method according to claim 26, wherein the activating reagent is selected from the group consisting of N-(3-dimethylaminopropyl)-N′-ethylcarbodiimide (EDC), N,N′-diisopropylcarbodiimide (DIC), and N,N′-dicyclohhexylcarbodiimide (DCC), N,N′-carbonyl diimidazole (CDI), t-butyl-ethylcarbodiimide and t-butyl-methylcarbodiimide.

28. Method according to claim 17, wherein the polynucleotide primer with one 2′ or 3′ terminal amino group is reacted with the nucleotide comprising a 5′ terminal activated phosphate ester or activated carboxylic ester, or with a nucleotide or a polynucleotide with an activated 5′ terminal phosphate or carboxy residue or wherein the polynucleotide primer with a 5′ terminal amino group is reacted with the nucleotide comprising one 2′ or 3′ terminal activated phosphate ester or activated carboxylic ester, or with a nucleotide or a polynucleotide with one 2′ or 3′ terminal activated phosphate or carboxy residue.

29. Method according to claim 17, comprising the step of annealing the polynucleotide primer to a single or double stranded polynucleotide template.

30. Method according to claim 29, wherein the polynucleotide template comprises at least a one nucleotide overhang 3′ and/or 5′ with respect to the polynucleotide primer.

31. Method according to claim 30, wherein the overhang has a length of four or more nucleotides.

32. Method according to claim 31, comprising the step of annealing a polynucleotide helper or a polynucleotide helper comprising a stacking residue to the polynucleotide template.

33. Method according to claim 32, wherein the stacking residue is selected from the group consisting of substituted or unsubstituted indole, napthol, anthraquinone, pyrene, a steroid ring system, bile acid, quinoline, quinolone, stilbene, an ethidium residue, an anthracene residue, and tetracene, which can be substituted with one or more residues selected from the group consisting of OH, SH, NH₂, F, Cl, Br and I.

34. Method according to claim 32, wherein the length of the nucleotide gap between the annealed polynucleotide helper or a polynucleotide helper comprising a stacking residue and the annealed polynucleotide primer is identical to the length of the nucleotide according to claims 1 to 16, or the length of the nucleotide or the polynucleotide comprising an activated phosphate or carboxy residue, which is coupled to the polynucleotide primer.

35. Method according to claim 32, wherein the length of the nucleotide gap between the annealed polynucleotide helper comprising a stacking residue and the polynucleotide primer is one nucleotide larger than the length of the nucleotide according to claims 1 to 16 or the length of the nucleotide or the polynucleotide comprising an activated phosphate or carboxy residue, which is coupled to the polynucleotide primer.

36. Method according to claim 30, wherein at least two nucleotides carrying different bases are included in step b).

37. Method according to claim 17, wherein the step of coupling the nucleotide or a nucleotide or polynucleotide comprising an activated phosphate or carboxy residue to the reacting the polynucleotide primer is repeated one or more times.

38. Method according to claim 17, further comprising the step of analyzing the reaction product of step b).

39. Method according to claim 38, wherein the analysis is carried out by mass spectrometry, mass sensing, radiometry, fluorescence spectroscopy or phosphorescence spectroscopy, electrophoresis, chromatography, or atomic force microscopy.

40. Method according to claim 17, further comprising the step of photo cleavage of the spacer.

41. Use of a template-directed non-enzymatic extension of a polynucleotide for the determination of the sequence of a polynucleotide template 5′ or 3′-terminal from an annealed polynucleotide primer.

42. Use according to claim 41, for the determination of single nucleotide polymorphisms (SNPs), point mutations, chromosomal rearrangements, base modification, in particular cytosine methylation, splice variants, deletions or loss of nucleobases.

43. The method of claim 17, used for the determination of the sequence of a polynucleotide template 5′ or 3′-terminal from an annealed polynucleotide primer.

44. Method according to claim 43, for the determination of single nucleotide polymorphisms (SNPs), point mutations, chromosomal rearrangements, base modification, in particular cytosine methylation, splice variants, deletions or loss of nucleobases.

45. Use according to claim 41, further comprising the use of a polynucleotide helper with or without a stacking residue.

46. Kit comprising at least one nucleotide according to claim 1 and a polynucleotide primer, with at least one 2′-, 3′- or 5′-terminal amino group.

47. Kit comprising at least one activating reagent and a nucleotide or polynucleotide comprising an activatable phosphate or carboxy residue.

48. Kit according to claim 47, further comprising a polynucleotide primer, with at least one 2′-, 3′- or 5′-terminal amino group.

49. Kit according to claim 47, wherein the activating reagent has a structure according to formula (XL) 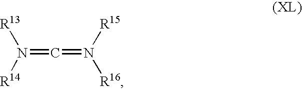
wherein
R¹³ and R¹⁶ independent of each other mean H, linear or branched, substituted or unsubstituted C₁ to C₁₀ alkyl, linear or branched C₁ to C₁₀ alkyl-NR¹⁷R¹⁸, wherein R¹⁷ and R¹⁸ independent of each other mean linear or branched substituted or unsubstituted C₁ to C₅ alkyl, C₃ to C₈ cycloalkyl, aryl, or heteroaryl;
R¹⁴ and R¹⁵ either mean a free electron pair or R¹³ and R¹⁴ and/or R¹⁵ and R¹⁶ together form a heteroaryl;
or is 2-fluoro pyridine; R^{V}—CO—Cl; or Z-SO₂—R^{V}, wherein R^{V} has the meaning saturated or unsaturated, C₁ to C₁₀ alkyl, aryl, heteroaryl, which can be substituted with one or more OH, SH, NH₂, F, Cl, Br, or I.

50. Kit according to claim 49, further comprising at least one catalyst with a structure according to formulas (XXXV) to (XXXXIV) 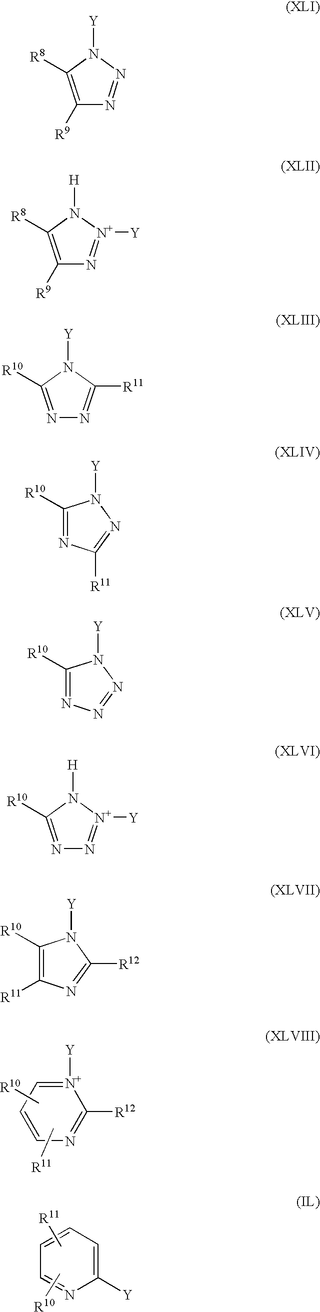 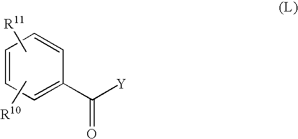
wherein
R⁸ and R⁹ independent of each other have the meaning H, OH, SH, NH₂, F, Cl, Br, I, saturated or unsaturated, linear or branched, unsubstituted or substituted C₁ to C₁₀ alkyl, or taken together form a saturated or unsaturated, unsubstituted or substituted mono, bi or polycyclic ring;
R¹⁰ and R¹¹ independent of each other have the meaning H, OH, SH, NH₂, F, Cl, Br, I, saturated or unsaturated, linear or branched, unsubstituted or substituted C₁ to C₁₀ alkyl, linear or branched C₁ to C₁₀ alkyl-NR¹⁹R²⁰, wherein R¹⁹ and R²⁰ independent of each other mean linear or branched substituted or unsubstituted C₁ to C₁₀ alkyl, C₃ to C₈ cycloalkyl, aryl, or heteroaryl;
R¹² has the meaning H, halogens H, OH, SH, NH₂, F, Cl, Br, I, CH₃, substituted methyl, saturated or unsaturated, linear or branched, unsubstituted or substituted C₂ to C₅ alkyl,
and Y is selected from the group consisting of H and OH.

51. Kit according to claim 50, wherein the catalyst is selected from the group consisting of imidazole, methylimidazole, benzimidazole, triazole, tetrazole, hydroxybenzotriazole, azahydroxybenzotriazole, chlorobenzotriazole, dimethylaminopyridine (DMP).

52. Kit according to claim 46 further comprising a polynucleotide helper or a polynucleotide helper comprising a stacking residue.
